(19) United States
(12) Patent Application Publication
(10) Pub. No.: US 2017/0165240 A1
(43) Pub. Date: Jun. 15, 2017

(54) NEW STRATEGIES FOR TREATING MELANOMA

(71) Applicants: Université Libre de Bruxelles, Bruxelles (BE); INSTITUT JULES BORDET, Bruxelles (BE)

(72) Inventors: Ghanem GHANEM, Drogenbos (BE); Fabrice JOURNÉ, Blaregnies (BE); Mohammad KRAYEM, Bruxelles (BE); Ahmad AWADA, Ternat (BE)

(73) Assignees: Université Libre de Bruxelles, Bruxelles (BE); Institut Jules Bordet, Bruxelles (BE)

(21) Appl. No.: 15/300,835

(22) PCT Filed: Apr. 1, 2015
(86) PCT No.: PCT/EP2015/057214

§ 371 Date: Sep. 30, 2016

(30) Foreign Application Priority Data
Apr. 1, 2014 (EP) PCT/EP2014/056546

(51) Int. Cl. A61K 31/439; A61K 31/5377; A61K 31/519; A61K 31/437

(52) U.S. Cl. CPC A61K 31/439; A61K 31/437; A61K 31/5377; A61K 31/519

(57) ABSTRACT

The present invention relates to a p53-activating agent capable of transferring wild-type tumor protein p53 (p53) from an inactive conformation into an active conformation capable of inducing apoptosis, for use in the treatment of melanoma, wherein said p53-activating agent is administered simultaneously or sequentially with a BRAF-inhibiting agent capable of inhibiting activity of serine/threonine-protein kinase B-Raf (BRAF) comprising an activating mutation.

### FIELD OF THE INVENTION

The invention is broadly situated in the medical field, more specifically in the field of treatment of melanoma. The invention allows countering the intrinsic and acquired resistance of melanoma cells with an activating BRAF mutation to specific BRAF inhibitors.

### BACKGROUND

Melanoma incidence and mortality are high in United States and Europe and recent epidemiologic data documented increases of its incidence in the last few decades (Garbe and Leiter, 2009, Clin. Dermatol., 27, 3-9). Estimated age-standardized incidence varies widely in Europe from 19.2 (per 100,000 persons and year) in Switzerland to 2.2 in Greece (10.5 in Belgium), probably depending on opportunities for early diagnosis and incomplete reporting, while melanoma mortality rate of 1.5 is rather similar across Europe (Forsea et al., 2012, Br. J. Dermatol., 167, 1124-1130). The 5-year survival rate for patients with early detection of localized melanoma is about 90%, while it drops to 60% and 15% for patients with regional and distant metastases, respectively (Siegel et al., 2012, CA. Cancer J. Clin., 62, 220-241).

Systemic treatment of metastatic melanoma is not always successful because of resistance to conventional chemotherapy. Dacarbazine, recorded by the FDA (Food and Drug Administration) in 1975, used to be, until recently, the standard for treatment of melanoma patients. It mainly induces partial responses in about 15% of all cases but without evidence of survival benefit (Serrone et al., 2000, J. Exp. Clin. Cancer Res. CR 19, 21-34). No other monotherapy has yet been shown to be more effective than dacarbazine. It may also be used in combination with other cytotoxic agents such as cisplatine, carmustine or vinblastine, sometimes yielding higher response rates (30-50%).

However, combined therapy is generally associated with greater toxicity and does not significantly increase survival (Lui et al., 2007, Cancer Treat. Rev., 33, 665-680).

Recently, strategy restoring an immune system's response to disease has been also developed, in particular, with the ipilimumab, an antibody raised against CTLA-4 molecule on T-cells. Ipilimumab improved overall survival in patients with previously treated metastatic melanoma. Furthermore, 9 of 15 responders (60%) in the ipilimumab only group maintained an objective response for at least 2 years (Hodi et al., 2010, N. Engl. J. Med., 363, 711-723). Ipilimumab in combination with dacarbazine improved overall survival versus dacarbazine plus placebo in patients with previously untreated metastatic melanoma (Robert et al., 2011, N. Engl. J. Med., 364, 2517-2526). Furthermore, a second immune therapy, an anti-PD1 antibody, produced objective responses in approximately 28% of patients with melanoma.

In parallel, increased understanding of the molecular events involved in melanoma development has led to the identification of novel targets and to the development of new targeted agents. Gene alterations identified in melanoma pointed to distinct molecular subsets of tumors with direct implications in therapeutic strategies. Among these, activating BRAF mutations occurring in 50 to 60% of melanomas (Davies et al., 2002, Nature, 417, 949-954) (V600E substitution represents about 90% of BRAF mutations) and NRAS mutations in 15 to 25% of melanomas (mutually exclusive with BRAF mutation) opened new therapeutic perspectives targeting the MAPK pathway (hyperactivated in 75% of melanomas) with, among other, ^{V600E}BRAF or MEK inhibitors. Vemurafenib, the first ^{V600E}BRAF kinase inhibitor approved by the FDA in 2011, improved rates of overall and progression-free survival compared to dacarbazine in patients with previously untreated ^{V600E}BRAF melanoma (Chapman et al., 2011, N. Engl. J. Med., 364, 2507-2516). Dabrafenib, a second ^{V600E}BRAF inhibitor, produced promising tumor shrinkage in patients with mutant BRAF metastatic melanoma and particularly with melanoma brain metastases, a frequent complication of metastatic melanoma (Falchook et al., 2012, Lancet, 379, 1893-1901; Hauschild et al., 2012, Lancet, 380, 358-365). Trametinib, a selective inhibitor of MEK1 and MEK2, also improved rates of progression-free survival and overall survival versus chemotherapy in patients with metastatic melanoma and V600E or V600K mutations in BRAF (Flaherty et al., 2012, N. Engl. J. Med., 367, 1694-1703). With evidence of persistent MEK phosphorylation despite BRAF inhibition, dabrafenib has been combined with trametinib leading to improved response rates and median progression-free survival compared with treatment using dabrafenib alone (Flaherty et al., 2012, N. Engl. J. Med., 367, 1694-1703). Among new MEK inhibitors in clinical development, pimasertib has been recently reported to be particularly promising in the case of patients with mutant NRAS melanoma (Akinleye et al., 2013, J. Hematol. Oncol., 6, 27).

Nevertheless, in spite of significant initial responses, resistance developed in almost all patients. Disease progression is observed in about 50% of patients on targeted therapy within 6 months of treatment initiation (Solit and Rosen, 2011, N. Engl. J. Med., 364, 772-774). Recent studies reported multiple mechanisms for recurrences: switches between pathways (Chapman et al., 2011, N. Engl. J. Med., 364, 2507-2516), activation/stabilization of CRAF (Heidorn et al., 2010, Cell, 140, 209-221), COT/MAP3K8 activation (Johannessen et al., 2010, Nature, 468, 968-972), appearance of new activating mutations in ^{C121S}MEK1 (Wagle et al., 2011, J. Clin. Oncol., 29, 3085-3096), dimerization of aberrantly spliced ^{V600E}BRAF (Poulikakos et al., 2011, Nature, 480, 387-390), or upregulation of receptor tyrosine kinase (Nazarian et al., 2010, Nature, 468, 973-977).

Therefore, further and/or improved treatment strategies are required to increase progression-free survival and improve complete response rates.

### SUMMARY OF THE INVENTION

The present inventors have unexpectedly realised that an agent capable of transferring wild type p53 from an inactive conformation thereof into an active conformation capable of inducing apoptosis sensitizes melanoma cells to an agent capable of inhibiting activity of BRAF comprising an activating mutation by breaking both intrinsic and acquired resistance. This fact can be advantageously exploited in various applications, including inter alia the treatment of melanoma. The inventors further demonstrated that said p53 reactivation may, in addition to its known proapoptotic activity, moderate PI3K/AKT signaling, whose activation is a major resistance mechanism to vemurafenib.

It is well documented that p53 is largely inactivated in melanoma by a variety of mechanisms, of which overexpression of MDM2 that currently is evaluated in the clinic as a target for therapy. However, in different mutant BRAF vemurafenib-resistant melanoma lines established by the inventors, MDM2 was only weakly expressed in one (MM133), while MDM4 is detected in another (MM054), suggesting other mechanisms causing p53 inactivation may be present in BRAF mutated lines. The inventors have now established that direct p53 reactivation, whatever is the inhibition mechanism or mutational status, emerges as a promising treatment strategy. In such BRAF mutated cells, Ser15 phosphorylation may be viewed as a marker of functional reactivation of p53 and the upregulation of the tumor suppressor PTEN, the specific phosphatase coupled to the kinase PI3K and decreases the transcription of p110α, the catalytic subunit of PI3K, consequently inhibiting the AKT pathway. This was observed not only in melanoma cells with intrinsic resistance to vemurafenib but also in melanoma cell-lines that acquired resistance to vemurafenib (due to prolonged treatment with vemurafenib).

Furthermore, the inventors compared p53-induced PI3K/AKT inhibition to that of two different PI3K or PI3K/mTOR inhibitors, each combined with vemurafenib. A clear advantage for the former in terms of growth inhibition and apoptosis promotion was shown in vemurafenib resistant melanoma cells, thus adding the benefit of inhibiting PI3K/AKT pathway to the known p53 effects on promoting apoptosis.

In summary, the present invention highlights the potential clinical benefit of combining MAPK inhibition to p53 reactivation in BRAF mutated melanoma. Unexpectedly, from various combinatorial modalities tested, targeting the MAPK and PI3K, signaling pathways through p53 reactivation or not, the direct activation of p53 (such as through PRIMA-1 ^{Met}) in combination with BRAF inhibitor (such as through vemurafenib) was the most cytotoxic for resistant melanoma cells.

FIG. 23 shows a simplified scheme illustrating the effect of combined BRAF inhibitor and p53 reactivation on melanoma cell survival. Mutant BRAF is inhibited by vemurafenib while PI3K/AKT pathway is inactivated by p53 activation using PRIMA-1^{Met}. In addition, p53 restoration induced apoptosis. Both drugs act in synergy to inhibit melanoma growth.

Accordingly, the invention provides the following aspects:

Aspect 1. A method of treating melanoma in a patient, comprising the step of administering to said patient, a therapeutically effective amount of a p53-activating agent capable of transferring wild-type tumor protein p53 (p53) from an inactive conformation into an active conformation capable of inducing apoptosis, simultaneously or sequentially with the administration of a BRAF-inhibiting agent capable of inhibiting activity of serine/threonine-protein kinase B-Raf (BRAF) comprising an activating mutation.

Aspect 2. The method according to aspect 1, wherein said p53-activating agent is administered after or before administration of said BRAF-inhibiting agent.

Aspect 3. The method according to aspect 1 or 2, wherein said p53-activating agent is administered simultaneously with the BRAF-inhibiting agent.

Aspect 4. The method according to any one of aspects 1 to 3, wherein said p53-activating agent is a compound having the structure of Formula I, or a pharmaceutically acceptable salt or prodrug thereof,

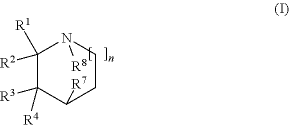

wherein

n is 0,1 or 2;

R¹ and R² are the same or different and are selected from —H, —CH₂-R⁵, —CH₂—O—R⁵, —CH₂—S—R⁵, —CH₂—NH—R⁵, —CO—R⁵, —CO—NH—R⁵, —CH₂—NH—CO—R⁵, —CH₂—O—CO—R⁵, —CH₂—NH—CO—NHR⁵, —CH₂—NH—CO—OR⁵, —CH₂—NH—CS—NHR⁵ and —CH₂—O—CO—NHR⁵; or R¹ and R² are together ═CH₂;

R³ and R⁴ are the same or different and are selected from —H, —OH, —SH, —NH2, —NHR⁵ and —O—OC—C₆H₅; or R³ and R⁴ together are ═O, ═S, =NH or ═NR⁵;

R⁵ represents the same or different groups selected from H, substituted or non-substituted C₁₋₁₀alkyl, C₂₋₁₀alkenyl, C₂₋₁₀alkynyl, substituted or non-substituted C₃₋₁₂cycloalkyl, substituted or non-substituted benzyl groups, substituted or non-substituted aryl or mono-, bi-, tricyclic unsubstituted or substituted heteroaromatic ring(s) with one or more heteroatoms and non-aromatic heterocycles wherein the substituents of the substituted groups are selected from C₁₋₁₀alkyl, C₂₋₁₀alkenyl, C₂₋₁₀alkynyl, halogen, substituted or non-substituted aryl, substituted or non-substituted heteroaromatic compounds, non-aromatic heterocycles, C₁₋₁₀alkyloxy, C₁₋₁₀alkylamino, C₂₋₁₀alkenylamino, C₂₋₁₀alkynylamino, COR⁶, CONR⁶ and COOR⁶;

R⁶ is selected from H, unsubstituted or substituted C₁₋₁₀alkyl, C₂₋₁₀alkenyl or alkynyl, benzyl, aryl, unsubstituted or substituted heteroaromatic rings with one or more heteroatoms and non-aromatic heterocycles;

R⁷ and R⁸ together form a bridging CH₂—CH₂ moiety; or R⁷ and R⁸ are both hydrogen, or wherein said p53-activating agent is CDB3, SCH529074, NSC319726, or CP-31398.

Aspect 5. The method according to any one of aspects 1 to 4, wherein said p53-activating agent is a compound selected from the group consisting of 2-hydroxymethyl-2-methoxymethylazabicyclo[2.2.2]octan-3-one, 2 ,2-bis(hyd roxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, and 2-(2,6-dichloro-9-methylenepurine)-3-quinuclidinone, or a pharmaceutically acceptable salt thereof.

Aspect 6. The method according to any one of aspects 1 to 5, wherein said BRAF-inhibiting agent is a compound having the structure of Formula III, or a pharmaceutically acceptable salt or prodrug thereof,

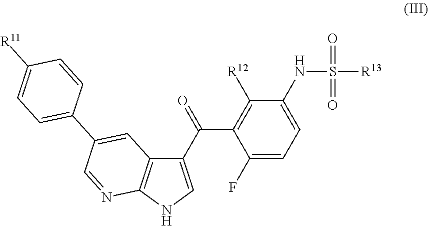

wherein

R¹¹ is selected from the group consisting of hydrogen, halogen, optionally substituted C₁₋₆alkyl, optionally substituted C₁₋₆alkenyl, optionally substituted C₁₋₆alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, and —S(O)₂R¹⁵;

R¹² is selected from the group consisting of hydrogen, fluoro and chloro;

R¹³ is selected from the group consisting of optionally substituted C₂₋₆alkyl, optionally substituted aryl, optionally substituted heteroaryl, and NR¹⁶R¹⁷;

R¹⁴ is selected from the group consisting of optionally substituted C₁₋₆-alkyl, optionally substituted C₁₋₆-alkenyl, provided, however, that when R¹⁴ is optionally substituted C₁₋₆-alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted C₁₋₆alkynyl, provided, however, that when R¹⁴ is optionally substituted C₁₋₆alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted cycloalkyl, optionally substituted heterocycloalkyl, optionally substituted aryl, and optionally substituted heteroaryl;

R¹⁵ is selected from the group consisting of hydrogen and optionally substituted C₁₋₆alkyl; and

R¹⁶ and R¹⁷ are independently hydrogen or optionally substituted C₁₋₆alkyl, or R¹⁶ and R¹⁷ combine with the nitrogen to which they are attached to form optionally substituted 5-6 membered heterocycloalkyl.

Aspect 7. The method according to any one of aspects 1 to 5, wherein said BRAF-inhibiting agent is a compound selected from the group consisting of N-(3-{[5-(4-chlorophenyl)-1 H-pyrrolo[2,3-b]pyridin-3-yl]carbonyl}-2,4-difluorophenyl)-propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl-[(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine.

Aspect 8. The method according to any one of aspects 1 to 7, wherein said p53-activating agent is 2-hydroxymethyl-2-methoxymethylazabicyclo[2.2.2]octan-3-one (PR1MA-1^{Met}) and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib); or

wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is SCH529074 (Demma M, et al., 2010, J Biol Chem. 285(14):10198-10212) and said BRAF-inhibiting agent is N-(3-{[5-(4-chlorophenyl)-1 H-pyrrolo[2,3-b]pyridin-3-yl]carbonyl1-2,4-difluorophenyl)-propane-1-sulfonamide (vemurafenib); or

wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is NSC319726 (Yu X. et al., 2012, Cancer Cell. 15;21(5):614-25) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib).

Aspect 9. The method according to any one of aspects 1 to 8, wherein said p53-activating agent and said BRAF-inhibiting agent are comprised in a composition or in a kit of parts, preferably in a pharmaceutical composition or in a pharmaceutical kit of parts.

Aspect 10. The method according to any one of aspects 1 to 9, wherein the melanoma comprises expression of BRAF comprising an activating mutation, preferably wherein the melanoma comprises (a) cell(s) comprising expression of ^{V600E/K}BRAF.

Aspect 11. The method according to any one of aspects 1 to 9, wherein the melanoma comprises (a) cell(s) with intrinsic or acquired resistance to said BRAF-inhibiting agent.

Aspect 12. A method of treating melanoma resistant to N-(3-{[5-(4-chlorophenyl)-1H-pyrrolo[2,3-b]pyridin-3-yl]carbonyl}-2,4-difluorophenyl)-propane-1-sulfonamide (vemurafenib) in a patient, comprising the administration of a therapeutically effective amount of a p53-activating agent capable of transferring wild-type tumor protein p53 from an inactive conformation into an active conformation capable of inducing apoptosis.

Aspect 13. The method according to aspect 12, wherein said resistance is pre-existing, or is acquired due to (chronic) treatment with N-(3-{[5-(4-chlorophenyl)-1H-pyrrolo[2,3-b]pyridin-3-yl]carbonyl}-2,4-difluorophenyl) propane-1-sulfonamide (vemurafenib).

Aspect 14. A method of treating melanoma in a patient, comprising the administration of a pharmaceutical composition comprising: a p53-activating agent capable of transferring wild-type p53 from an inactive conformation thereof into an active conformation capable of inducing apoptosis and a BRAF-inhibiting agent capable of inhibiting activity of BRAF comprising an activating mutation, for use in treating melanoma.

Aspect 15. The method according to aspect 14, wherein said p53-activating agent is 2-hydroxymethyl-2-methoxymethylazabicyclo[2.2.2]octan-3-one (PRIMA-1^{Met}and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib); or

wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is SCH529074 (Demma M, et al., 2010, J Biol Chem. 285(14):10198-10212) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is NSC319726 (Yu X. et al., 2012, Cancer Cell. 15;21(5):614-25) and said BRAF-inhibiting agent is N-(3-{[5-(4-chlorophenyl)-1 H-pyrrolo[2,3-b]pyridin-3-yl]carbonyl}-2,4-difluorophenyl)-propane-1-sulfonamide (vemurafenib).

Aspect 16. The method according to any one of aspects 1 to 15, wherein said p53-activating agent and said BRAF-inhibiting agent are comprised in a composition or in a kit of parts, preferably in a pharmaceutical composition or in a pharmaceutical kit of parts.

Aspect 17. The method according to any one of aspects 1 to 15, wherein said p53-activating agent may be administered simultaneously or sequentially with said BRAF-inhibiting agent and with a MEK-inhibiting agent capable of inhibiting activity of mitogen-activated protein kinase kinase 1 (MEK 1) and/or mitogen-activated protein kinase kinase 2 (MEK2). Non-limiting examples of MEK-inhibitors are trametinib (also known as GSK1120212), pimasertib, selumetinib (also known as AZD6244), MEK162, PD-325901, Cobimetinib (also known as GDC-0973 or XL-518), or CI-1040.

Aspect 18. The method according to aspect 17, wherein said p53-activating agent, said BRAF-inhibiting agent and said MEK-inhibiting agent are comprised in a composition or in a kit of parts, preferably in a pharmaceutical composition or in a pharmaceutical kit of parts.

Aspect 19. A p53-activating agent capable of transferring wild-type tumor protein p53 (p53) from an inactive conformation into an active conformation capable of inducing apoptosis, for use in the treatment of melanoma, wherein said p53-activating agent is administered simultaneously or sequentially with a BRAF-inhibiting agent capable of inhibiting activity of serine/threonine-protein kinase B-Raf (BRAF) comprising an activating mutation.

Aspect 20. The p53-activating agent for use according to aspect 19, wherein said p53-activating agent is administered after administration of said BRAF-inhibiting agent.

Aspect 21. The p53-activating agent for use according to aspect 19 or 20, wherein said p53-activating agent is administered before administration of the BRAF-inhibiting agent.

Aspect 22. The p53-activating agent for use according to any one of aspects 19 to 21, wherein said p53-activating agent is a compound having the structure of Formula I, or a pharmaceutically acceptable salt or prodrug thereof,

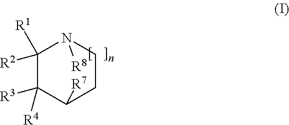

wherein

n is 0,1 or 2;

R¹ and R² are the same or different and are selected from —H, —CH₂-R⁵, —CH₂—O—R⁵, —CH₂—S—R⁵, —CH₂—NH—R⁵, —COO—R⁵, —CO—NH—R⁵, —CH₂—NH—CO—R⁵, —CH₂—O—CO—R⁵, —CH₂—NH—CO—NHR⁵, —CH₂—NH—CO—OR⁵, —CH₂—NH—CS—NHR⁵ and —CH₂—O—CO—NHR⁵; or R¹ and R² are together ═CH₂;

R³ and R⁴ are the same or different and are selected from —H, —OH, —SH, —NH2, —NHR⁵ and —O—CO—C₆H₅⁻; or R³ and R⁴ together are ═O, ═S, ═NH or ═NR⁵;

R⁵ represents the same or different groups selected from H, substituted or non-substituted C₁₋₁₀alkyl, C₂₋₁₀alkenyl, C₂₋₁₀alkynyl, substituted or non-substituted C₃₋₁₂cycloalkyl, substituted or non-substituted benzyl groups, substituted or non-substituted aryl or mono-, bi-, tricyclic unsubstituted or substituted heteroaromatic ring(s) with one or more heteroatoms and non-aromatic heterocycles wherein the substituents of the substituted groups are selected from C₁₋₁₀alkyl, C₂₋₁₀alkenyl, C₂₋₁₀alkynyl, halogen, substituted or non-substituted aryl, substituted or non-substituted heteroaromatic compounds, non-aromatic heterocycles, C₁₋₁₀alkyloxy, C₁₋₁₀alkylamino, C₂₋₁₀alkenylamino, C₂₋₁₀alkynylamino, COR⁶, CONR⁶ and COOR⁶;

R⁶ is selected from H, unsubstituted or substituted C₁₋₁₀alkyl, C₂₋₁₀alkenyl or alkynyl, benzyl, aryl, unsubstituted or substituted heteroaromatic rings with one or more heteroatoms and non-aromatic heterocycles;

R⁷ and R⁸ together form a bridging CH₂—CH₂ moiety; or R⁷ and R⁸ are both hydrogen; or wherein said p53-activating agent is CDB3, SCH529074, NSC319726, or CP-31398.

Aspect 23. The p53-activating agent for use according to any one of aspects 19 to 22, wherein said p53-activating agent is a compound selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, and 2-(2,6-dichloro-9-methylenepurine)-3-quinuclidinone, or a pharmaceutically acceptable salt thereof.

Aspect 24. The p53-activating agent for use according to any one of aspects 19 to 23, wherein said BRAF-inhibiting agent is a compound having the structure of Formula III, or a pharmaceutically acceptable salt or prodrug thereof,

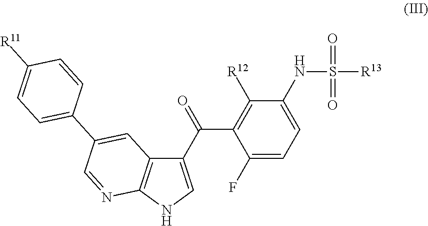

wherein

R¹¹ is selected from the group consisting of hydrogen, halogen, optionally substituted C₁₋₆alkyl, optionally substituted C₁₋₆alkenyl, optionally substituted C₁₋₆alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, and —S(O)₂R¹⁵;

R¹² is selected from the group consisting of hydrogen, fluoro and chloro;

R¹³ is selected from the group consisting of optionally substituted C₂₋₆alkyl, optionally substituted aryl, optionally substituted heteroaryl, and NR¹⁶R¹⁷;

R¹⁴ is selected from the group consisting of optionally substituted C₁₋₆-alkyl, optionally substituted C₁₋₆-alkenyl, provided, however, that when R¹⁴ is optionally substituted C₁₋₆-alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted C₁₋₆alkynyl, provided, however, that when R¹⁴ is optionally substituted C₁₋₆alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)N R¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted cycloalkyl, optionally substituted heterocycloalkyl, optionally substituted aryl, and optionally substituted heteroaryl;

R¹⁵ is selected from the group consisting of hydrogen and optionally substituted C₁₋₆alkyl; and

R¹⁶ and R¹⁷ are independently hydrogen or optionally substituted C₁₋₆alkyl, or R¹⁶ and R¹⁷ combine with the nitrogen to which they are attached to form optionally substituted 5-6 membered heterocycloalkyl.

Aspect 25. The p53-activating agent for use according to any one of aspects 19 to 24, wherein said BRAF-inhibiting agent is a compound selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine.

Aspect 26. The p53-activating agent for use according to any one of aspects 19 to 25, wherein said p53-activating agent is 2-hydroxymethyl-2-methoxymethylazabicyclo[2.2.2]octan-3-one (PRIMA-1^{Met}) and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib); or

wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is SCH529074 (Demma M, et al., 2010, J Biol Chem. 285(14):10198-10212) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is NSC319726 (Yu X. et al., 2012, Cancer Cell. 15;21(5):614-25) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib).

Aspect 27. The p53-activating agent for use according to any one of aspects 19 to 26, wherein said p53-activating agent and said BRAF-inhibiting agent are comprised in a composition or in a kit of parts, preferably in a pharmaceutical composition or in a pharmaceutical kit of parts.

Aspect 28. The p53-activating agent for use according to any one of aspects 19 to 26, wherein said p53-activating agent may be administered simultaneously or sequentially with said BRAF-inhibiting agent and with a MEK-inhibiting agent capable of inhibiting activity of mitogen-activated protein kinase kinase 1 (MEK 1) and/or mitogen-activated protein kinase kinase 2 (MEK2).

Aspect 29. The p53-activating agent for use according to aspect 28, wherein said p53-activating agent said BRAF-inhibiting agent and said MEK-inhibiting agent are comprised in a composition or in a kit of parts, preferably in a pharmaceutical composition or in a pharmaceutical kit of parts.

Aspect 30. The p53-activating agent for use according to any one of aspects 19 to 29, wherein the melanoma comprises expression of BRAF comprising an activating mutation, preferably wherein the melanoma comprises (a) cell(s) comprising expression of ^{V600E/K}BRAF.

Aspect 31. The p53-activating agent for use according to any one of aspects 19 to 30, wherein the melanoma comprises (a) cell(s) with intrinsic or acquired resistance to said BRAF-inhibiting agent.

Aspect 32. A p53-activating agent capable of transferring wild-type tumor protein p53 (p53) from an inactive conformation into an active conformation capable of inducing apoptosis, for use in the treatment of melanoma resistant to N-(3-{[5-(4-chlorophenyl)-1H-pyrrolo[2,3-b]pyridin-3-yl]carbonyl}-2,4-difluorophenyl) propane-1-sulfonamide (vemurafenib).

Aspect 33. The p53-activating agent for use according to aspect 32, wherein said resistance is pre-existing, or is acquired due to (chronic) treatment with N-(3-{[5-(4-chlorophenyl)-1H-pyrrolo[2,3-b]pyridin-3-yl]carbonyl}-2,4-difluorophenyl)-propane-1-sulfonamide (vemurafenib).

Aspect 34. A kit of parts or a composition, preferably a pharmaceutical kit of parts or a pharmaceutical composition, comprising a p53-activating agent capable of transferring wild-type p53 from an inactive conformation thereof into an active conformation capable of inducing apoptosis and a BRAF-inhibiting agent capable of inhibiting activity of BRAF comprising an activating mutation, for use in treating melanoma.

Aspect 35. The kit of parts or the composition according to aspect 33, wherein said p53-activating agent is 2-hydroxymethyl-2-methoxymethylazabicyclo[2.2.2]octan-3-one (PRIMA-1^{Met}) and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib).

Aspect 36. The kit of parts or the composition according to anyone of aspects 34 or 35, additionally comprising a MEK-inhibiting agent capable of inhibiting activity of mitogen-activated protein kinase kinase 1 (MEK 1) and/or mitogen-activated protein kinase kinase 2 (MEK2). Non-limiting examples of MEK-inhibitors are trametinib (also known as GSK1120212), pimasertib, selumetinib (also known as AZD6244), MEK162, PD-325901, Cobimetinib (also known as GDC-0973 or XL-518), or CI-1040

Aspect 37. A method for determining resistance of melanoma to a BRAF-inhibiting agent as defined according to anyone of aspects 19 to 33, in a subject, wherein the method comprises the steps of:

(i) preparing a cell culture from a sample of the melanoma obtained from the subject, and

(ii) determining the cytotoxicity of cells of the cell culture to said BRAF-inhibiting agent, wherein the melanoma is intrinsically resistant to said BRAF-inhibiting agent when the IC50 of the cells is at least 10 μM, and wherein the melanoma is sensitive to said BRAF-inhibiting agent when the IC50 of the cells is less than 10 μM.

Aspect 38. The method according to aspect 47, for predicting the development of resistance to said BRAF-inhibiting agent in a melanoma initially sensitive to said BRAF-inhibiting agent, wherein the method further comprises the steps of:

(iii) treating the melanoma cell culture by chronic exposure with increasing concentrations of said BRAF-inhibiting agent during at least about 4 weeks, and

(iv) determining the cytotoxicity of cells of the cell culture to said BRAF-inhibiting agent after said treatment, wherein the melanoma has acquired resistance to the BRAF-inhibiting agent when the IC50 of the cells is at least 10 μM.

Aspect 39. The method according to aspect 47 or 48, wherein the melanoma sample obtained from the subject originates from a metastasis of the subject, for example from skin, lymph node, mucosa, liver, or gastrointestinal tract.

Aspect 40. A method for predicting responsiveness of melanoma resistant to a BRAF-inhibiting agent as defined according to anyone of aspects 19 to 33, to treatment with a p53-activating agent as defined according to anyone of aspects 19 to 33 in combination said BRAF-inhibiting agent in a subject, comprising the steps of:

(i) preparing a cell culture from a sample of the melanoma obtained from the subject,

(ii) determining the expression of one or more of p53, Phosphatase and tensin homolog (PTEN), and phospho-Protein kinase B (pAKT) in cells of the cell culture, and

(iii) predicting that the melanoma is responsive to treatment with said p53-activating agent in combination with said BRAF-inhibiting agent, if the cells express low p53, low PTEN, and/or high pAkt compared with expression of the respective proteins in cells of a cell culture prepared from a melanoma sensitive to said BRAF-inhibiting agent.

Aspect 41. A method for predicting responsiveness of melanoma to treatment with a p53-activating agent as defined according to anyone of aspects 19 to 33 in combination with a BRAF-inhibiting agent defined according to anyone of aspects 19 to 33 in a subject, comprising the steps of:

(i) preparing a cell culture from a sample of the melanoma obtained from the subject,

(ii) administering said p53-activating agent in combination with said BRAF-inhibiting agent,

(iii) administering said BRAF-inhibiting agent alone as a control treatment,

(iv) determining the cytotoxicity of cells of the cell culture to said p53 activating agent and said BRAF-inhibiting agent and to said BRAF-inhibiting agent alone, and

(v) predicting that the melanoma is responsive to treatment with said p53-activating agent in combination with said BRAF-inhibiting agent, if the cytotoxicity of said p53-activating agent in combination with said BRAF-inhibiting agent to the cells is higher compared to the cytotoxicity of cells treated with said BRAF-inhibiting agent alone.

Aspect 42. The method of treatment according to anyone of aspects 1 to 18, wherein the subject is a subject with a melanoma responsive to treatment with the p53-activating agent in combination with the BRAF-inhibiting agent, as determined by the method according to aspect 40 or 41.

Aspect 43. The p53-activating agent for use according to any one of aspects 19 to 33, wherein the subject is a subject with a melanoma responsive to treatment with the p53-activating agent in combination with the BRAF-inhibiting agent, as determined by the method according to aspect 40 or 41.

Aspect 44. Use of a p53-activating agent capable of transferring wild-type tumor protein p53 (p53) from an inactive conformation into an active conformation capable of inducing apoptosis, for the manufacture of a medicament for treatment of melanoma, wherein said p53-activating agent is administered simultaneously or sequentially with a BRAF-inhibiting agent capable of inhibiting activity of serine/threonine-protein kinase B-Raf (BRAF) comprising an activating mutation.

Aspect 45. The use according to aspect 44, wherein said p53-activating agent is administered after administration of said BRAF-inhibiting agent.

Aspect 46. The use according to aspect 44 or 45, wherein said p53-activating agent is administered before administration of the BRAF-inhibiting agent.

Aspect 47. The use according to any one of aspects 44 to 46, wherein said p53-activating agent is a compound having the structure of Formula I, or a pharmaceutically acceptable salt or prodrug thereof,

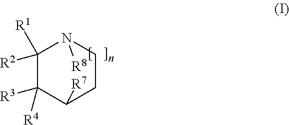

wherein

n is 0,1 or 2;

R¹ and R² are the same or different and are selected from —H, —CH₂—R⁵, —CH₂—O—R⁵, —CH₂—S—R⁵, —CH₂—NH—R⁵, —COO—R⁵, —CO—NH—R⁵, —CH₂—NH—CO—R⁵, —CH₂—O—CO—R⁵, —CH₂—NH—CO—NHR⁵, —CH₂—NH—CO—OR⁵, —CH₂—NH—CS—NHR⁵ and —CH₂—O—CO—NHR⁵; or R¹ and R² are together ═CH₂;

R³ and R⁴ are the same or different and are selected from —H, —OH, —SH, —NH2, —NHR⁵ and —O—CO—C₆H₅; or R³ and R⁴ together are ═O, ═S, ═NH or ═NR⁵;

R⁵ represents the same or different groups selected from H, substituted or non-substituted C₁₋₁₀alkyl, C₂₋₁₀alkenyl, C₂₋₁₀alkynyl, substituted or non-substituted C₃₋₁₂cycloalkyl, substituted or non-substituted benzyl groups, substituted or non-substituted aryl or mono-, bi-, tricyclic unsubstituted or substituted heteroaromatic ring(s) with one or more heteroatoms and non-aromatic heterocycles wherein the substituents of the substituted groups are selected from C₁₋₁₀alkyl, C₂₋₁₀alkenyl, C₂₋₁₀alkynyl, halogen, substituted or non-substituted aryl, substituted or non-substituted heteroaromatic compounds, non-aromatic heterocycles, C₁₋₁₀alkyloxy, C₁₋₁₀alkylamino, C₂₋₁₀alkenylamino, C₂₋₁₀alkynylamino, COR⁶, CONR⁶ and COOR⁶;

R⁶ is selected from H, unsubstituted or substituted C₁₋₁₀alkyl, C₂₋₁₀alkenyl or alkynyl, benzyl, aryl, unsubstituted or substituted heteroaromatic rings with one or more heteroatoms and non-aromatic heterocycles;

R⁷ and R⁸ together form a bridging CH₂—CH₂ moiety; or R⁷ and R⁸ are both hydrogen; or wherein said p53-activating agent is CDB3, SCH529074, NSC319726, or CP-31398.

Aspect 23. The p53-activating agent for use according to any one of aspects 19 to 22, wherein said p53-activating agent is a compound selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, and 2-(2,6-dichloro-9-methylenepurine)-3-quinuclidinone, or a pharmaceutically acceptable salt thereof.

Aspect 48. The use according to any one of aspects 44 to 47, wherein said BRAF-inhibiting agent is a compound having the structure of Formula III, or a pharmaceutically acceptable salt or prodrug thereof,

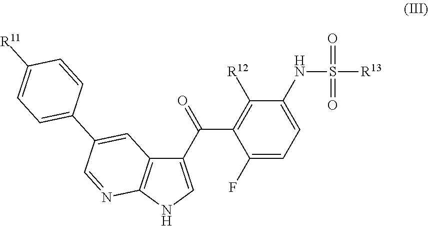

wherein

R¹¹ is selected from the group consisting of hydrogen, halogen, optionally substituted C₁₋₆alkyl, optionally substituted C₁₋₆alkenyl, optionally substituted C₁₋₆alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, and —S(O)₂R¹⁵;

R¹² is selected from the group consisting of hydrogen, fluoro and chloro;

R¹³ is selected from the group consisting of optionally substituted C₂₋₆alkyl, optionally substituted aryl, optionally substituted heteroaryl, and NR¹⁶R¹⁷;

R¹⁴ is selected from the group consisting of optionally substituted C₁₋₆-alkyl, optionally substituted C₁₋₆-alkenyl, provided, however, that when R¹⁴ is optionally substituted C₁₋₆-alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵O(S)N H₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted C₁₋₆alkynyl, provided, however, that when R¹⁴ is optionally substituted C₁₋₆alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR15C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted cycloalkyl, optionally substituted heterocycloalkyl, optionally substituted aryl, and optionally substituted heteroaryl;

R¹⁵ is selected from the group consisting of hydrogen and optionally substituted C₁₋₆alkyl; and

R¹⁶ and R¹⁷ are independently hydrogen or optionally substituted C₁₋₆alkyl, or R¹⁶ and R¹⁷ combine with the nitrogen to which they are attached to form optionally substituted 5-6 membered heterocycloalkyl.

Aspect 49. The use according to any one of aspects 44 to 48, wherein said BRAF-inhibiting agent is a compound selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine.

Aspect 50. The use according to any one of aspects 44 to 49, wherein said p53-activating agent is 2-hydroxymethyl-2-methoxymethylazabicyclo[2.2.2]octan-3-one (PRIMA-1^{Met}) and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib); or

wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or wherein said p53-activating agent is SCH529074 (Demma M, et al., 2010, J Biol Chem. 285(14):10198-10212) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or

wherein said p53-activating agent is NSC319726 (Yu X. et al., 2012, Cancer Cell. 15;21(5):614-25) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib).

Aspect 51. The use according to any one of aspects 44 to 50, wherein said p53-activating agent and said BRAF-inhibiting agent are comprised in a composition or in a kit of parts, preferably in a pharmaceutical composition or in a pharmaceutical kit of parts.

Aspect 52. The use according to any one of aspects 44 to 51, wherein said p53-activating agent may be administered simultaneously or sequentially with said BRAF-inhibiting agent and with a MEK-inhibiting agent capable of inhibiting activity of mitogen-activated protein kinase kinase 1 (MEK 1) and/or mitogen-activated protein kinase kinase 2 (MEK2).

Aspect 53. The use according to aspect 52, wherein said p53-activating agent said BRAF-inhibiting agent and said MEK-inhibiting agent are comprised in a composition or in a kit of parts, preferably in a pharmaceutical composition or in a pharmaceutical kit of parts.

Aspect 54. The use according to any one of aspects 44 to 53, wherein the melanoma comprises expression of BRAF comprising an activating mutation, preferably wherein the melanoma comprises (a) cell(s) comprising expression of ^{V600E/K}BRAF.

Aspect 55. The p53-activating agent for use according to any one of aspects 44 to 54, wherein the melanoma comprises (a) cell(s) with intrinsic or acquired resistance to said BRAF-inhibiting agent.

Aspect 56. Use of a p53-activating agent capable of transferring wild-type tumor protein p53 (p53) from an inactive conformation into an active conformation capable of inducing apoptosis, for use in the treatment of melanoma resistant to N-(3-{[5-(4-chlorophenyl)-1H-pyrrolo[2,3-b]pyridin-3-yl]carbonyl}-2,4-difluorophenyl) propane-1-sulfonamide (vemurafenib).

Aspect 57. The use according to aspect 56, wherein said resistance is pre-existing, or is acquired due to (chronic) treatment with N-(3-{[5-(4-chlorophenyl)-1H-pyrrolo[2,3-b]pyridin-3-yl]carbonyl}-2,4-difluorophenyl) propane-1-sulfonamide (vemurafenib).

The present invention offers a real opportunity to overcome both intrinsic and acquired resistance of melanoma to BRAF-inhibiting agents such as vemurafenib. Indeed, the data suggest that a combination of a BRAF inhibiting agent (such as vemurafenib) with a p53-activating agent (such as PRIMA-1^{Met}) allows breaking resistance to the BRAF-inhibiting agent.

Melanoma primary cultures that can be established from patients, enabling studying the associated cellular mechanisms, can hence be viewed as tools for tailoring the treatment of the patient. In such a way, the drug or combination that has the best chance to be affective can be determined in vitro, outside the patient. Such a primary melanoma culture can be used to mimic and depict, in vitro, the escape pathways that the cells could switch on to survive the control mechanisms, long before the clinical situation actually occurs in the patient in vivo. It hence allows predicting the reaction of the melanoma cells towards the BRAF-inhibitors.

These and further aspects and preferred embodiments of the invention are described in the following sections and in the appended claims. The subject-matter of the appended claims is hereby specifically incorporated in this specification.

### BRIEF DESCRIPTION OF DRAWINGS

FIG. 1 represents two graphs illustrating the effect of vemurafenib on cell proliferation in a ^{V600E}BRAF melanoma cell line sensitive to vemurafenib (MM074, left graph) and in a ^{V600E}BRAF melanoma cell line with intrinsic resistance to vemurafenib (MM043, right graph).

FIG. 2 represents two graphs illustrating the effect of vemurafenib on apoptosis in a ^{V600E}BRAF melanoma cell line sensitive to vemurafenib (MM074, left graph) and in a ^{V600E}BRAF melanoma cell line with intrinsic resistance to vemurafenib (MM043, right graph).

FIG. 3 represents Western blots illustrating the evaluation of key effectors of signalling pathways involved in cell survival in a ^{V600E}BRAF melanoma cell line sensitive to vemurafenib (MM074, left panels) and in a ^{V600E}BRAF melanoma cell line with intrinsic resistance to vemurafenib (MM043, right panels).

FIG. 4 represents two graphs illustrating the effect of vemurafenib (full line) or the combination of vemurafenib with the p53 activator PRIMA-1^{Met} (dashed line) on cell proliferation in a ^{V600E}BRAF melanoma cell line sensitive to vemurafenib (MM074, left graph) and in a ^{V600E}BRAF melanoma cell line with intrinsic resistance to vemurafenib (MM043, right graph).

FIG. 5 represent two graphs illustrating the effect of vemurafenib (Vemu), PRIMA-1^{Met}, or the combination of vemurafenib with PRIMA-1^{Met} on apoptosis in a ^{V600E}BRAF melanoma cell line sensitive to vemurafenib (MM074, left graph) and in a ^{V600E}BRAF melanoma cell line with intrinsic resistance to vemurafenib (MM043, right graph).

FIG. 6 represents Western blots illustrating the evaluation of p53, PTEN, and pAKT in a ^{V600E}BRAF melanoma cell line sensitive to vemurafenib (MM074) and in a ^{V600E}BRAF melanoma cell line with intrinsic resistance to vemurafenib (MM043). β-actin was evaluated as a control.

FIG. 7 represents a schematic overview of acquiring resistance of the parental ^{V600E}BRAF melanoma cell line MM074 to MM074-R by chronic treatment with vemurafenib.

FIG. 8 represents a graph illustrating the effect of vemurafenib on cell proliferation in a parental ^{V600E}BRAF melanoma cell line (MM074, dashed line) and the cell line with acquired resistance (MM074-R, full line).

FIG. 9 represents Western blots illustrating the evaluation of p53, PTEN, and pAKT in a parental ^{V600E}BRAF melanoma cell line (MM074, left panels) and the cell line with acquired resistance (MM074-R, right panels). β-actin was evaluated as a control.

FIG. 10 represents a graph illustrating the effect of vemurafenib (full line) or the combination of vemurafenib with the p53 activator PRIMA-1^{Met} (dashed line) on cell proliferation in a ^{V600E}BRAF melanoma cell line with acquired resistance to vemurafenib (MM074-R).

FIG. 11 represents a graph illustrating the effect of vemurafenib (Vemu), PRIMA-1^{Met}, or the combination of vemurafenib with PRIMA-1^{Met} on apoptosis in a ^{V600E}BRAF melanoma cell line with acquired resistance to vemurafenib (MM074-R).

FIG. 12 Shows the combination of BRAF inhibition and p53 reactivation in a panel of vemurafenib-sensitive (MM074) and resistant (all others) ^{V600E}/KBRAF melanoma lines. Effect of vemurafenib (vemu) and PRIMA-1^{Met} alone or in combination for 3 days on cell proliferation (crystal violet staining) and for 2 days on apoptosis (annexin V-positive cells). PRIMA-1^{Met} concentrations are IC10 values as calculated for each line. Data are presented as means+SD (n=3) compared to untreated and single drug treated cells, *** p<0.001 (Student's t-test).

FIG. 13 Shows the effect of vemurafenib on ERK and AKT phosphorylation in a panel of ^{V600E}/KBRAF melanoma lines with intrinsic resistance to vemurafenib. Representative Western blots illustrating the evolution of (A) ERK and (B) AKT phosphorylation in melanoma cells exposed to increasing concentrations of vemurafenib (0.1, 1 and 10 μM) for 24 hours. Ratio of phosphorylated protein over total protein were calculated from densitometry evaluation.

FIG. 14 Shows the constitutive expression levels of MDM2 and MDM4 in melanoma cell-lines. Representative Western blots showing the basal expression of MDM2/4 in a panel of nine ^{V600E/K}BRAF melanoma lines in comparison with positive control (^{WT}BRAF/^{WT}NRAS HBL melanoma cells exposed to 25 μM proteasome inhibitor MG-132 for 1 hour). β-actin is used as loading control.

FIG. 15 Shows the effect of vemurafenib on cell apoptosis in the sensitive MM074 and the resistant MM043 cells in relation with the constitutive status of key effectors involved in MAPK and p53/PI3K/AKT pathways. (A) Apoptosis induced by cell exposure to increasing concentrations (0.01-10 μM) of vemurafenib (vemu) for 2 days as evaluated by the percentage of annexin V-positive cells. Data are presented as means+SD (n=3) compared to untreated cells (CTR). ***p<0.001 (Student's t-test). (B) Constitutive phosphorylation and expression levels of key proteins of MAPK and p53/PI3K/AKT pathways assessed by Western blotting in MM074 and MM043 cell lines. β-actin is used as loading control.

FIG. 16 Shows the effect of vemurafenib on key proteins of MAPK and PI3K/AKT pathways in the sensitive MM074 and the resistant MM043 lines. (A) Representative Western blots illustrating the evolution of ERK and AKT phosphorylation and p110α and PTEN expression in melanoma cells exposed to increasing concentrations of vemurafenib (vemu) (0.01-10 μM) for 24 hours. (B) Densitometric analyses of the immunoreactive bands. ERK and AKT phosphorylation levels were corrected with ERK and AKT total protein expression, p110α and PTEN expression levels were corrected with the beta-actin expression. Results present the means of 2 independent experiments.

FIG. 17 Shows the combination of BRAF inhibition and p53 reactivation in cells with intrinsic resistance to vemurafenib (MM043) compared to sensitive cells (MM074). (A) Effect of PRIMA-1Met (20, 25 or 50 μM for 24 hours) alone or combined to 0.1 μM vemurafenib (vemu) on p53, p53 Ser15, p21, p110α, PTEN, pAKT and AKT as evaluated by Western blotting. beta-actin is used as loading control. (B,C) Effect of vemurafenib (0.01-100 μM) alone or in combination with the p53 activator PRIMA-1^{Met} (20 μM) on cell proliferation. Data are expressed as means+SD (n=3) compared to untreated cells (CTR).

FIG. 18 Shows the combination of BRAF and PI3K/AKT pathway inhibition in cells with intrinsic resistance to vemurafenib (MM043) compared to sensitive cells (MM074). (A) Effect of 5 μM LY294002 and 0.1 μM PF-04691502 exposure on AKT phosphorylation for 30 minutes as evaluated by Western blotting. (B,C) Effect of increasing concentrations (0.01-100 μM) of vemurafenib (vemu) for 3 days alone or in combination with 5 μM LY294002 or 0.1 μM PF-04691502 on cell proliferation. Data are expressed as means+SD (n=3) compared to untreated cells (CTR). (D,E) Apoptosis induced by cell exposure to 0.1 μM vemurafenib and/or 5 μM LY294002 or 0.1 μM PF-04691502 for 2 days. Data are presented as means+SD (n=3) compared to untreated cells, ** p<0.01 (Student's t-test).

FIG. 19 Shows the combination of BRAF inhibition and p53 reactivation in cells with acquired resistance to vemurafenib. (A) Effect of vemurafenib (vemu) (0.01-100 μM) for 3 days on cell proliferation in MM074 line (sensitive, parental) and MM074-R line with acquired resistance to vemurafenib. Data are expressed as means+SD (n=3) compared to untreated cells (CTR). (B) Western blots illustrating the evaluation of p53, p21, p110α, PTEN, pAKT and AKT in parental sensitive line (MM074) and in line with acquired resistance (MM074-R). beta-actin is used as loading control. (C) Effect of vemurafenib (0.01-100 μM) alone or in combination with 5 μM LY294002, 0.1 μM PF-04691502 or 40 μM PRIMA-1^{Met} on cell proliferation. Data represent means+SD (n=3) compared to untreated cells (CTR). (D) Evaluation of apoptosis in MM074-R cells exposed to 10 μM vemurafenib and/or 5 μM LY294002, 0.1 μM PF-04691502 or 40 μM PRIMA-1^{Met} for 2 days. Data are presented as means+SD (n=3) compared to untreated cells, *p<0.05, **p<0.01, ***p<0.001 (Student's t-test). (E) Effect of PRIMA-1^{Met} (50 and 75 μM for 24 hours) on p53, p53 Ser15, p21, p110α, PTEN, pAKT and AKT in cells with acquired resistance to vemurafenib (MM074-R) as assessed by Western blotting. beta-actin is used as loading control.

FIG. 20 represents a graph illustrating the in vivo effect of control treatment (diamonds), or treatment with vemurafenib (squares), PRIMA-1^{Met} (triangles), or the combination of vemurafenib with PRIMA-1^{Met} (circles) on the growth of ^{V600E}BRAF melanoma cells with intrinsic resistance to vemurafenib (MM043) by showing the tumor volume (in mm³) as a function of the duration of the treatment (in days).

FIG. 21 Shows the effect of vemurafenib and PRIMA-1^{Met} combination on inhibition of human melanoma tumor growth in nude mice. (A-C) Growth curves for tumors grafted in mice and treated as control (DMSO), with vemurafenib (vemu) (45 mg/kg), with PRIMA-1^{Met} (50 mg/kg), or with the combination of both drugs. Tumors rose from cells with high sensitivity (MM074), with intrinsic resistance (MM043) and with acquired resistance to vemurafenib (MM074-R). Data are presented as means tumor volumes (mm³)+SEM compared to DMSO-treated cells, ***p<0.001 (two-way ANOVA). (D-F) Animal weight measured every two days during the whole experiments. Data are presented as means+SEM.

FIG. 22 represents a schematic overview of a Phase I study to determine the safety and tolerability of the combination regimen of vemurafenib and PRIMA-1^{Met}, and to define dose limiting toxicity (DLT) and maximum tolerated dose (MTD) of the combination regimen of vemurafenib and PRIMA-1^{Met}. PET-CT: Positron emission tomography-computed tomography; PR: Partial Response; CR: Complete Response; SD: Stable Disease; MPD: Metabolic Progressive Disease.

FIG. 23 represents a schematic overview of a flow sheet of the development of an in vitro mechanistic model to predict sensitivity or resistance of patients to vemurafenib alone or in combination with PRIMA-1^{Met}. SK/LN metastasis: establishment of primary ^{V600E}BRAF cell lines from skin (SK) or lymph node (LN) melanoma metastases.

FIG. 24 shows a simplified scheme illustrating the effect of combined BRAF inhibitor and p53 reactivator on melanoma cell survival. Mutant BRAF is inhibited by vemurafenib while PI3K/AKT pathway is inactivated by p53 activation using PRIMA-1^{Met}. In addition, p53 restoration induced apoptosis. Both drugs act in synergy to inhibit melanoma growth.

### DETAILED DESCRIPTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The term “p53” encompasses the tumor suppressor protein known as p53, or tumor protein 53, cellular tumor antigen p53, phosphoprotein p53, or tumor suppressor p53. It is a human protein encoded by the TP53 gene. It is a protein that is crucial in regulating the cell-cycle in all multicellular organisms. p53 can activate DNA repair proteins, it can arrest growth by holding the cell cycle at the G1/S regulation point on DNA damage recognition, and can induce apoptosis when the DNA damage is beyond repair. In many cancers, p53 is inactivated, either due to a mutation, or due to suppression through e.g. a viral oncogen, or through overexpression of another interacting protein. In the present invention, it was established that in many melanoma cells, that are, or become resistant to treatment with BRAF inhibitors, the p53 protein is not mutated (i.e., it is wild-type), but yet is suppressed or kept inactive. By combining the BRAF inhibiting agent with a p53 activator, this resistance is markedly reduced, ameliorating greatly the effect on the cancer cells.

As defined herein, the term “p53-activating agent” or “p53 activator” encompasses all agents capable of transferring wild-type p53 from an inactive conformation into an active conformation which is capable of inducing apoptosis. This direct p53 reactivation whatever is the inhibition mechanism or mutational status, emerges as an alternative promising strategy. Some p53-binding molecules are known that not only rescue mutant p53 but also activate the function of wild-type p53 by affecting its conformation. The mechanism of p53 reactivation by these molecules is not completely understood, but it has been suggested that their binding to the DNA binding domain of p53 may induce its phosphorylation and a conformational block that prevents the docking of p53 inhibitors. Specific examples of such agents are compounds having the structure of Formula I, or a pharmaceutically acceptable salt or prodrug thereof,

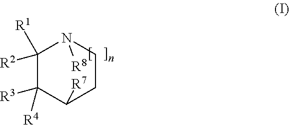

wherein

n is 0,1 or 2;

R¹ and R² are the same or different and are selected from —H, —CH₂—R⁵, —CH₂—O—R⁵, —CH₂—S—R⁵, —CH₂—NH—R⁵, —COO—R⁵, —CO—NH—R⁵, —CH₂—NH-CO—R⁵, —CH₂—O—CO—R⁵, —CH₂—NH—CO—NHR⁵, —CH₂—NH—CO—OR⁵, —CH₂—NH—CS—NHR⁵ and —CH₂—O—CO—NHR⁵; or R¹ and R² are together ═CH₂;

R³ and R⁴ are the same or different and are selected from —H, —OH, —SH, —NH2, —NHR⁵ and —O—CO—C₆H₅; or R³ and R⁴ together are ═O, ═S, ═NH or ═NR⁵;

R⁵ represents the same or different groups selected from H, substituted or non-substituted C₁₋₁₀alkyl, C₂₋₁₀alkenyl, C₂₋₁₀alkynyl, substituted or non-substituted C₃₋₁₂cycloalkyl, substituted or non-substituted benzyl groups, substituted or non-substituted aryl or mono-, bi-, tricyclic unsubstituted or substituted heteroaromatic ring(s) with one or more heteroatoms and non-aromatic heterocycles wherein the substituents of the substituted groups are selected from C₁₋₁₀alkyl, C₂₋₁₀alkenyl, C₂₋₁₀alkynyl, halogen, substituted or non-substituted aryl, substituted or non-substituted heteroaromatic compounds, non-aromatic heterocycles, C₁₋₁₀alkyloxy, C₁₋₁₀alkylamino, C₂₋₁₀alkenylamino, C₂₋₁₀alkynylamino, COR⁶, CONR⁶ and COOR⁶;

R⁶ is selected from H, unsubstituted or substituted C₁₋₁₀alkyl, C₂₋₁₀alkenyl or alkynyl, benzyl, aryl, unsubstituted or substituted heteroaromatic rings with one or more heteroatoms and non-aromatic heterocycles;

R⁷ and R⁸ together form a bridging CH₂—CH₂ moiety; or R⁷ and R⁸ are both hydrogen.

Further specific examples of p53 (re)activating agents as defined herein are: CDB3 (Issaeva N et al., 2003, Proc Natl Acad Sci USA. 100(23):13303-13307); SCH529074 (Demma M, et al., 2010, J Biol Chem. 285(14):10198-10212); CP-31398 (Luu Y and Li G, 2002, J Invest Dermatol, 119(5):1207-1209; Luu Yet al., 2002 Exp Cell Res, 276(2):214-222.) and a member of the thiosemicarbazone family such as NSC319726, NSC319725, and NSC328784, preferably NSC319726 (Yu X. et al., 2012, Cancer Cell. 15;21(5):614-25).

The term “BRAF” or “BRAF protein” as used herein refers to the human protein B-Raf, encoded by human gene BRAF. The gene is also referred to as proto-oncogene B-Raf, and v-Raf murine sarcoma viral oncogene homolog B1. The protein is also known as serine/threonine-protein kinase B-Raf. B-Raf is a 766-amino acid protein comprising three conserved Raf kinase domains: a Ras-GTP-binding self-regulatory domain (CR1), a serine-rich hinge region (CR2), and a catalytic protein kinase domain (CR3) phosphorylating protein substrates such as the AKT-1 protein. The BRAF protein is a member of the Raf kinase family of growth signal transduction protein kinases and is involved in regulating the MAP kinase/ERK signaling pathways involved in (amongst others) cell proliferation and cell differentiation. The BRAF protein is tightly regulated in normal cells, but can be mutated in cancerous cells, such as melanoma. In 90% of such cases, said mutation leads to a valine (V) being substituted by another amino acid at codon 600 (V600-mutant) of the amino acid sequence of BRAF. Examples are e.g. valine substitutions by glutamate (E) or lysine (K), resulting in respectively V600E and V600K BRAF mutants. These mutations leads to a constitutive active BRAF protein, referred herein as “mutated BRAF protein comprising an activating mutation” or “BRAF comprising an activating mutation”.

The term “BRAF-inhibiting agent” or “BRAF inhibitor”, as used herein, encompasses all compounds, agents, or compositions that are capable of inhibiting the activity of the mutated BRAF protein comprising an activating mutation as defined herein. Exemplary agents are: Vemurafenib (N-(3-{[5-(4-chlorophenyl)-1H-pyrrolo[2,3-b]pyridin-3-yl]carbonyl}-2,4-difluorophenyl) propane-1-sulfonamide, Zelboraf, PLX4032), or its analogues as defined by Formula III as taught herein, and PLX4720 (N-[3-(5-chloro-1H-pyrrolo[2,3-b]pyridine-3-carbonyl)-2,4-difluorophenyl]propane-1-sulfonamide), both adenine-analogs, binding the ATP binding site of (over)activated BRAF, not of inactive BRAF. This selectivity is important, since the agent will normally only inhibit proliferation of cells with unregulated (over)active BRAF, i.e. in cancer cells. Alternative BRAF inhibitors can be selected from the group consisting of GDC-0879 ((E)-2,3-Dihydro-5-[1-(2-hydroxyethyl)-3-(4-pyridinyl)-1H-pyrazol-4-yl]-1H-inden-1-one oxime), Sorafenib (4-[4-[[4-chloro-3-(trifluoromethyl)phenyl]carbamoylamino]phenoxy]-N-methyl-pyridine-2-carboxamide, Nexavar, BAY43-9006, tosylated or not). Dabrafenib (N-{3-[5-(2-aminopyrimidin-4-yl)-2-tert-butyl-1,3-thiazol-4-yl]-2-fluorophenyl}-2,6-difluorobenzenesulfonamide, Tafinlar), LGX818 (methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate), and RAF265 ({1-methyl-5-[2-(5-trifluoromethyl-1H-imidazol-2-yl)-pyridin-4-yloxy]-1H-benzoimidazol-2-yl}-(4-trifluoromethyl-phenyl)-amine).

Sorafenib (Bay43-9006) disables the B-Raf kinase domain by locking the enzyme in its inactive form.

The term “vemurafenib” refers to a compound of Formula IIIa.

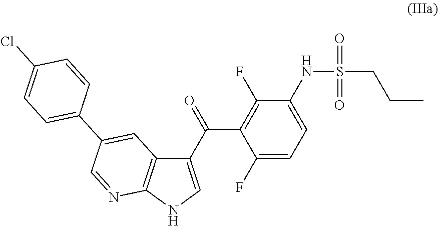 [SMILES: CCCS(=O)(=O)Nc1ccc(F)c(C(=O)c2c[nH]c3ncc(-c4ccc(Cl)cc4)cc23)c1F]

The terms “vemurafenib”, “Vemu”, or “N-(3-{[5-(4-chlorophenyl)-1H-pyrrolo[2,3-b]pyridin-3-yl]carbonyl}-2,4-difluorophenyl)propane-1-sulfonamide” may be used interchangeably.

The term “dabrafenib” refers to a compound of Formula IV.

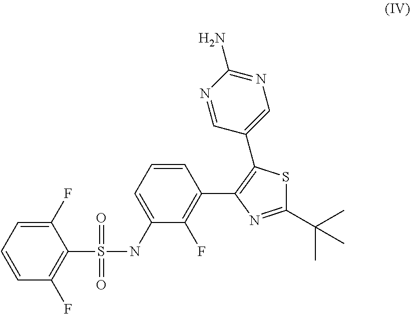 [SMILES: CC(C)(C)c1nc(-c2cccc(NS(=O)(=O)c3c(F)cccc3F)c2F)c(-c2cnc(N)nc2)s1]

The terms “dabrafenib”, “Tafinlar” or “N-{3-[5-(2-aminopyrimidin-4-yl)-2-tert-butyl-1,3-thiazol-4-yl]-2-fluorophenyl}-2,6-difluorobenzenesulfonamide” may be used interchangeably.

The term “sorafenib” refers to a compound of Formula V.

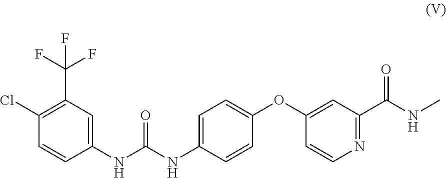 [SMILES: CNC(=O)c1cc(Oc2ccc(NC(=O)Nc3ccc(Cl)c(C(F)(F)F)c3)cc2)ccn1]

The terms “sorafenib”, “Nexavar”, or “4-[4-[[4-chloro-3-(trifluoromethyl)phenyl]carbamoylamino]phenoxy]-N-methyl-pyridine-2-carboxamide” may be used interchangeably.

The term “RAF265” refers to a compound of Formula VI.

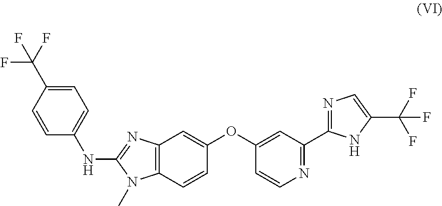 [SMILES: Cn1c(Nc2ccc(C(F)(F)F)cc2)nc2cc(Oc3ccnc(-c4ncc(C(F)(F)F)[nH]4)c3)ccc21]

The terms “RAF265”, or {1-methyl-5-[2-(5-trifluoromethyl-1H-imidazol-2-yl)-pyridin-4-yloxy]-1H-benzoimidazol-2-yl}-(4-trifluoromethyl-phenyl)-amine” or “1-methyl-5-[[2-[5-(trifluoromethyl)-1H-imidazol-2-yl]-4-pyridyl]oxy]-N-[4-(trifluoromethyl)phenyl]benzimidazol-2-amine” may be used interchangeably.

The term “LGX818” refers to a compound of Formula VII.

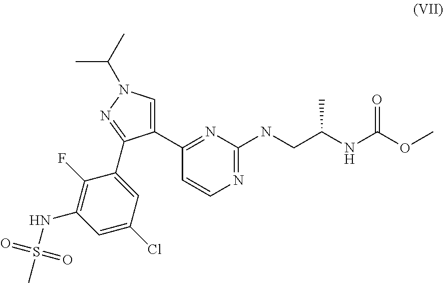 [SMILES: COC(=O)N[C@@H](C)CNc1nccc(-c2cn(C(C)C)nc2-c2cc(Cl)cc(NS(C)(=O)=O)c2F)n1]

The terms “LGX818” or “methyl N-[(1S)-2-[[4-[3-[5-chloro-2-fluoro-3-(methanesulfonamido)phenyl]-1-isopropyl-pyrazol-4-yl]pyrimidin-2-yl]amino]-1-methyl-ethyl]carbamate” or “methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate” may be used interchangeably.

The term “GDC-0879” refers to a compound of Formula VIII.

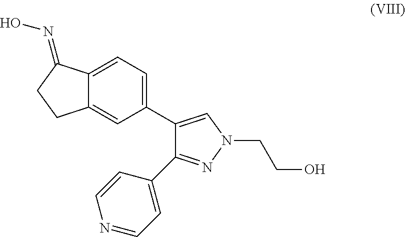 [SMILES: OCCn1cc(-c2ccc3c(c2)CC/C3=N\O)c(-c2ccncc2)n1]

The terms “GDC-0879”, “(E)-2,3-Dihydro-5-[1-(2-hydroxyethyl)-3-(4-pyridinyl)-1H-pyrazol-4-yl]-1H-inden-1-one oxime”, or “5-(1-(2-hydroxyethyl)-3-pyridin-4-yl-1H-pyrazol-4-yl)-2,3-dihydroinden-1-one oxime” may be used interchangeably.

In certain embodiments, said BRAF-inhibiting agent may be selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine.

In certain embodiments, said BRAF-inhibiting agent may be N-(3-{[5-(4-chlorophenyl)-1H-pyrrolo[2,3-b]pyridin-3-yl]carbonyl}-2,4-difluorophenyl) propane-1-sulfonamide (vemurafenib) or N-{3-[5-(2-aminopyrimidin-4-yl)-2-tert-butyl-1,3-thiazol-4-yl]-2-fluorophenyl}-2,6-difluorobenzenesulfonamide (dabrafenib).

In certain embodiments, said BRAF-inhibiting agent may be N-(3-{[5-(4-chlorophenyl)-1H-pyrrolo[2,3-b]pyridin-3-yl]carbonyl}-2,4-difluorophenyl) propane-1-sulfonamide (vemurafenib).

In certain embodiments, said BRAF-inhibiting agent as defined herein may be a compound of Formula III, or a pharmaceutically acceptable salt, prodrug, tautomer, or isomer thereof,

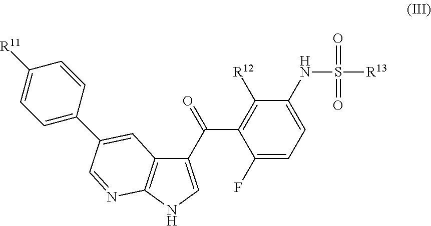

wherein

R¹¹ is selected from the group consisting of hydrogen, halogen, optionally substituted C₁₋₆-alkyl, optionally substituted C₁₋₆-alkenyl, optionally substituted C₁₋₆-alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR¹⁴, —SR″, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, and —S(O)₂R¹⁵;

R¹² is selected from the group consisting of hydrogen, fluoro and chloro;

R¹³ is selected from the group consisting of optionally substituted C₂₋₆alkyl, optionally substituted aryl, optionally substituted heteroaryl, and NR¹⁶R¹⁷;

R¹⁴ is selected from the group consisting of optionally substituted C₁₋₆-alkyl, optionally substituted C₁₋₆-alkenyl, provided, however, that when R¹⁴ is optionally substituted C₁₋₆-alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —N R¹⁵C(S)N H₂, —NR¹⁵C(S)NR¹⁵R¹⁴, —N R¹⁵S(O)₂N H₂, —NR¹⁵S(O)₂NR¹⁵R¹⁴, —S(O)R¹⁵, or —S(O)₂R¹⁵, optionally substituted C₁₋₆-alkynyl, provide, however, that when R¹⁴ is optionally substituted C₁₋₆-alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁶S(O)₂NR¹⁶R¹⁴, —S(O)R¹⁵, or —S(O)₂R¹⁵, optionally substituted cycloalkyl, optionally substituted heterocycloalkyl, optionally substituted aryl, and optionally substituted heteroaryl;

R¹⁵ is selected from the group consisting of hydrogen and optionally substituted C₁₋₆alkyl; and

R¹⁶ and R¹⁷ are independently hydrogen or optionally substituted C₁₋₆-alkyl, or R¹⁶ and R¹⁷ combine with the nitrogen to which they are attached to form optionally substituted 5-6 membered heterocycloalkyl.

The term “MEK” encompasses mitogen-activated protein kinase kinase (also known as MAP2K) which is a kinase enzyme which phosphorylates mitogen-activated protein kinase (MAPK). There are seven genes: MAP2K1 (or MEK1); MAP2K2 (or MEK2); MAP2K3 (or MKK3); MAP2K4 (or MKK4); MAP2K5 (or MKK5); MAP2K6 (or MKK6); and MAP2K7 (or MKK7). Mitogen-activated protein kinase kinase 1 (MEK1) and mitogen-activated protein kinase kinase 2 (MEK2) are activators of ERK.

The term “MEK-inhibiting agent” or “MEK inhibitor”, as used herein, encompasses all compounds, agents, or compositions that are capable of inhibiting the activity of MEK1 and/or MEK2. MEK1 (or MAP2K1) and MEK2 (MAP2K2) are also referred to as MEK1/2 (or MAP2K1/K2). MEK1/2 are dual-specificity threonine/tyrosine kinases that play key roles in the activation of the RAS/RAF/MEK/ERK pathway and are often upregulated in a variety of tumor cell types such as in melanoma, in particular in melanoma comprising expression of BRAF comprising an activating mutation.

Non-limiting examples of MEK inhibitors are trametinib (also known as GSK1120212), pimasertib, selumetinib (also known as AZD6244), MEK162, PD-325901, Cobimetinib (also known as GDC-0973 or XL-518), or CI-1040.

The term “trametinib” refers to a compound of Formula IX.

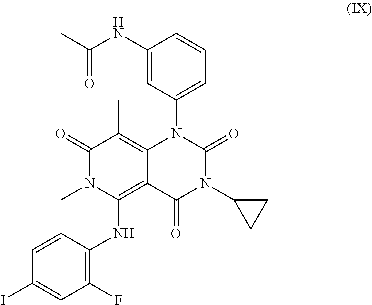 [SMILES: CC(=O)Nc1cccc(-n2c(=O)n(C3CC3)c(=O)c3c(Nc4ccc(I)cc4F)n(C)c(=O)c(C)c32)c1]

The terms “trametinib”, “N-[3-[3-cyclopropyl-5-(2-fluoro-4-iodoanilino)-6,8-dimethyl-2,4,7-trioxopyrido[4,3-d]pyrimidin-1-yl]phenyl]acetamide”, “GSK1120212”, “871700-17-3”, “JTP-74057”, “JTP 74057”, “GSK-1120212”, “Mekinist”, “JTP74057”, “JTP-74057”, or “871700-17-3” may be used interchangeably.

The term “pimasertib” refers to a compound of Formula X.

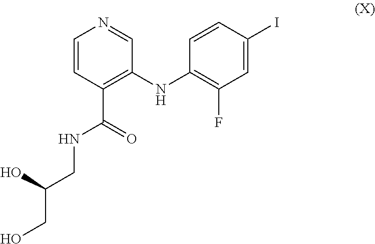 [SMILES: O=C(NC[C@H](O)CO)c1ccncc1Nc1ccc(I)cc1F]

The terms “pimasertib”, “N-[(2S)-2,3-dihydroxypropyi]-3-(2-fluoro-4-iodoanilino)pyridine-4-carboxamide”, “1236699-92-5”, “AS-703026”, “AS703026”, “AS 703026”, “MSC1936369B”, “AS-703026”, “MSC1936369B”, or “AS703026” may be used interchangeably.

The term “selumetinib” refers to a compound of Formula XI.

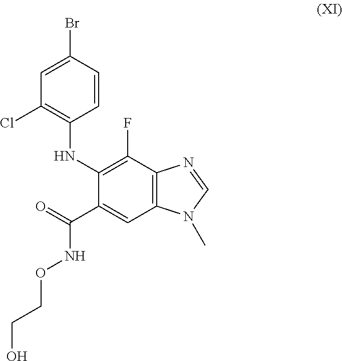 [SMILES: Cn1cnc2c(F)c(Nc3ccc(Br)cc3Cl)c(C(=O)NOCCO)cc21]

The terms “selumetinib”, “6-(4-bromo-2-chloroanilino)-7-fluoro-N-(2-hydroxyethoxy)-3-methylbenzimidazole-5-carboxamide”, “606143-52-6”, “AZD6244”, “AZD-6244”, or “ARRY-142886” may be used interchangeably.

The term “MEK162” refers to a compound of Formula XII.

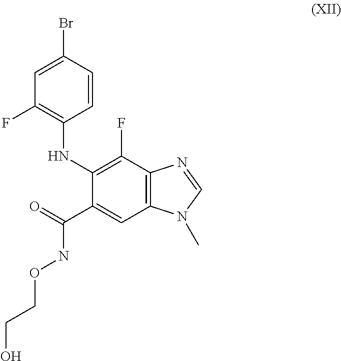 [SMILES: Cn1cnc2c(F)c(Nc3ccc(Br)cc3F)c(C(=O)NOCCO)cc21]

The terms “MEK162”, “6-(4-bromo-2-fluoroanilino)-7-fluoro-N-(2-hydroxyethoxy)-3-methylbenzimidazole-5-carboxamide”, “606143-89-9”, “ARRY 162”, “ARRY-162”, “ARRY-438162”, “ARRY 162”, “ARRY 438162”, “cc-455”, “MEK 162”, “MEK-162”, “QCR-138”, “ARRY-162”, or “MEK-162” may be used interchangeably.

The term “PD-325901” refers to a compound of Formula XIII.

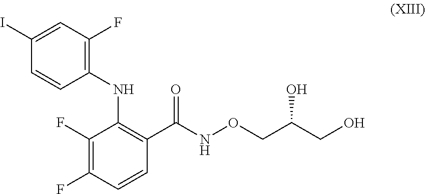 [SMILES: O=C(NOC[C@H](O)CO)c1ccc(F)c(F)c1Nc1ccc(I)cc1F]

The terms “PD-325901”, “N-[(2R)-2,3-dihydroxypropoxy]-3,4-difluoro-2-(2-fluoro-4-iodoanilino)benzamide”, “391210-10-9”, “PD0325901”, “PD-0325901”, “S1036_Selleck”, “PD325901”, “PD 325901”, “CHEMBL507361, or “ZINC03938683” may be used interchangeably.

The term “cobimetinib” refers to a compound of formula XIV.

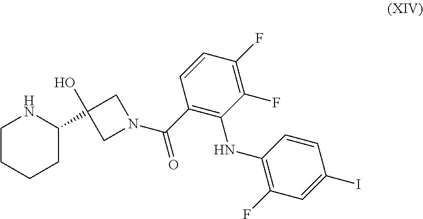 [SMILES: O=C(c1ccc(F)c(F)c1Nc1ccc(I)cc1F)N1CC(O)([C@@H]2CCCCN2)C1]

The terms “cobimetenib”, “[3,4-difluoro-2-(2-fluoro-4-iodoanilino)phenyl]-[3-hydroxy-3-[(2S)-piperidin-2-yl]azetidin-1-yl]methanone”, “GDC-0973”, “XL-518”, “XL518”, “GDC 0973”, “934660-93-2”, “XL 518”, “CHEMBL2146883”, or “GDC0973” may be used interchangeably.

The term “CI-1040” refers to a compound of Formula XV.

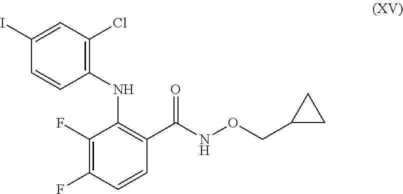 [SMILES: O=C(NOCC1CC1)c1ccc(F)c(F)c1Nc1ccc(I)cc1Cl]

The terms “CI-1040”, “2-(2-chloro-4-iodoanilino)-N-(cyclopropylmethoxy)-3,4-difluorobenzamide”, “212631-79-3”, “PD184352”, “PD 184352”, “PD-184352”, “AG-E-55891”, or “NCGC00189074-01” may be used interchangeably.

In certain embodiments, the MEK-inhibiting agent may be selected from the group consisting of N-[3-[3-cyclopropyl-5-(2-fluoro-4-iodoanilino)-6,8-dimethyl-2,4,7-trioxopyrido[4,3-d]pyrimidin-1-yl]phenyl]acetamide (trametinib), N-[(2S)-2,3-dihydroxypropyl]-3-(2-fluoro-4-iodoanilino)pyridine-4-carboxamide (pimasertib), 6-(4-bromo-2-chloroanilino)-7-fluoro-N-(2-hydroxyethoxy)-3-methylbenzimidazole-5-carboxamide (selumetinib), 6-(4-bromo-2-fluoroanilino)-7-fluoro-N-(2-hydroxyethoxy)-3-methylbenzimidazole-5-carboxamide (MEK162), N-[(2R)-2,3-dihydroxypropoxy]-3,4-difluoro-2-(2-fluoro-4-iodoanilino)benzamide (PD-325901), [3,4-difluoro-2-(2-fluoro-4-iodoanilino)phenyl]-[3-hydroxy-3-[(2S)-piperidin-2-yl]azetidin-1-yl]methanone (cobimetenib), and 2-(2-chloro-4-iodoanilino)-N-(cyclopropylmethoxy)-3,4-difluorobenzamide (CI-1040), or a pharmaceutically acceptable salt thereof.

In certain embodiments, the MEK-inhibiting agent may be N-[3-[3-cyclopropyl-5-(2-fluoro-4-iodoanilino)-6,8-dimethyl-2,4,7-trioxopyrido[4,3-d]pyrimidin-1-yl]phenyl]acetamide (trametinib) or N-[(2S)-2,3-dihydroxypropyl]-3-(2-fluoro-4-iodoanilino)pyridine-4-carboxamide (pimasertib), or a pharmaceutically acceptable salt thereof.

When describing the agents or compounds as taught herein, the terms used are to be construed in accordance with the following definitions, unless a context dictates otherwise.

For pharmaceutical use, the agents or compounds as taught herein may be used as a free acid or base, and/or in the form of a pharmaceutically acceptable acid-addition and/or base-addition salt (e.g. obtained with non-toxic organic or inorganic acid or base), in the form of a hydrate, solvate and/or complex, and/or in the form or a pro-drug or pre-drug, such as an ester. As used herein and unless otherwise stated, the term “solvate” includes any combination which may be formed by any unit or compound as taught herein with a suitable inorganic solvent (e.g. hydrates) or organic solvent, such as but not limited to alcohols, ketones, esters and the like. Such salts, hydrates, solvates, etc. and the preparation thereof will be clear to the skilled person; reference is for instance made to the salts, hydrates, solvates, etc. described in U.S. Pat. No. 6,372,778, U.S. Pat. No. 6,369,086, U.S. Pat. No. 6,369,087 and U.S. Pat. No. 6,372,733.

Whenever the term “substituted” is used in the present invention, it is meant to indicate that one or more hydrogens on the atom indicated in the expression using “substituted” is replaced with a selection from the indicated group, provided that the indicated atom's normal valency is not exceeded, and that the substitution results in a chemically stable compound, i.e., a compound that is sufficiently robust to survive isolation to a useful degree of purity from a reaction mixture, and formulation into a therapeutic agent.

Where groups may be substituted, such groups may be substituted with one or more, such as one, two, or three substituents. Substituents may be selected from but not limited to functional group such as hydroxyl, alkyl, alkoxy, amine, sulfide, silyl, carboxylic acid, halogen, aryl, etc.

The term “alkyl”, as a group or part of a group, refers to a hydrocarbyl group of Formula CₙH₂ₙ₊₁ wherein n is a number of at least 1. Alkyl groups may be linear, or branched and may be substituted as indicated herein. Generally, the alkyl groups comprise from 1 to 10 carbon atoms, preferably from 1 to 6 carbon atoms, more preferably 1, 2, 3, 4, 5, 6 carbon atoms. When a subscript is used herein following a carbon atom, the subscript refers to the number of carbon atoms that the named group may contain. For example, the term “C₁₋₁₀alkyl”, as a group or part of a group, refers to a hydrocarbyl group of Formula CₙH₂ₙ₊₁ wherein n is a number ranging from 1 to 10. For example, C₁₋₁₀alkyl includes all linear, or branched alkyl groups having 1 to 10 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers, heptyl and its isomers, octyl and its isomers, nonyl and its isomers, decyl and its isomers and the like. For example, C₁₋₆alkyl includes all linear, or branched alkyl groups having 1 to 6 carbon atoms, and thus includes for example methyl, ethyl, n-propyl, i-propyl, 2-methyl-ethyl, butyl and its isomers (e.g. n-butyl, i-butyl and t-butyl); pentyl and its isomers, hexyl and its isomers.

The term “C₂₋₁₀alkenyl” as a group or part of a group, refers to an unsaturated hydrocarbyl group, which may be linear, branched or cyclic, comprising one or more carbon-carbon double bonds, and comprising between 2 and 10 carbon atoms, preferably between 2 and 6 carbon atoms, more preferably between 2 and 4 carbon atoms, still more preferably between 2 and 3 carbon atoms. Non-limiting examples of alkenyl groups are ethenyl, 2-propenyl, 2-butenyl, 3-butenyl, 2-pentenyl and its isomers, 2-hexenyl and its isomers, 2,4-pentadienyl and the like.

The term “C₂₋₁₀alkynyl” as a group or part of a group, refers to a class of monovalent unsaturated hydrocarbyl groups, wherein the unsaturation arises from the presence of one or more carbon-carbon triple bonds, and comprising between 2 and 10 carbon atoms, preferably between 2 and 6 carbon atoms, more preferably between 2 and 4 carbon atoms, still more preferably between 2 and 3 carbon atoms. Non-limiting examples of alkynyl groups are ethynyl, 2-propynyl, 2-butynyl, 3-butynyl, 2-pentynyl and its isomers, 2-hexynyl and its isomers and the like.

The term “C₃₋₁₂cycloalkyl”, as a group or part of a group, refers to a cyclic alkyl group, that is a monovalent, saturated, hydrocarbyl group having 1 or more cyclic structure, and comprising from 3 to 12 carbon atoms, more preferably from 3 to 9 carbon atoms, more preferably from 3 to 6 carbon atoms, still more preferably from 5 to 6 carbon atoms. Cycloalkyl includes all saturated hydrocarbon groups containing 1 or more rings, including monocyclic or bicyclic groups. The further rings of multi-ring cycloalkyls may be fused, bridged, and/or joined through one or more spiro atoms. The term “C₃₋₆cycloalkyl”, as used herein, refers to a cyclic alkyl group comprising from 3 to 6 carbon atoms, more preferably from 5 to 6 carbon atoms. Non-limiting examples of C₃₋₆cycloalkyl groups include cyclopropyl, cyclobutyl, cyclopentyl, cyclohexyl. Cycloalkyl groups may also be considered to be a subset of homocyclic rings discussed hereinafter.

The term “homocyclic ring” as a group or part of a group, refers to a ring wherein the ring atoms comprise only carbon atoms. Non limiting examples of homocyclic rings include cycloalkyl, cycloalkenyl, with cycloalkyl being preferred. Where a ring carbon atom is replaced with a heteroatom, preferably nitrogen, oxygen of sulfur, the heteroatom-containing ring resultant from such a replacement is referred to herein as a heterocyclic ring or heterocycle. More than one carbon atom in a ring may be replaced so forming heterocyclic ring or heterocycle having a plurality of heteroatoms.

The terms “heterocycle” or “heterocyclic ring” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms.

Non limiting exemplary heterocycles include aziridinyl, oxiranyl, thiiranyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1 H-pyrrolizinyl, tetrahydro-1,1-dioxothiophenyl, N-formylpiperazinyl, and morpholin-4-yl.

As used herein; the term “non-aromatic heterocycle” or “heterocycloalkyl” means a non-aromatic cyclic group containing one or more heteroatom(s) preferably selected from N, O and S, such as a cyclic amino group such as pyrrolidinyl, piperidyl, piperazinyl, morpholinyl or a cyclic ether such as tetrahydrofuranyl, monosaccharide.

The term “benzyl” as a group or part of a group, refers to a group having the Formula —CH₂C₆H₅.

The term “aryl”, as a group or part of a group, refers to a polyunsaturated, aromatic hydrocarbyl group having a single ring (i.e. phenyl) or multiple aromatic rings fused together (e.g. naphthalene), or linked covalently, typically containing 6 to 12 atoms; wherein at least one ring is aromatic. The aromatic ring may optionally include one to two additional rings (cycloalkyl, heterocyclyl, or heteroaryl) fused thereto. Non-limiting examples of suitable aryl include C₆₋₁₀aryl, more preferably C₆₋₈aryl. Non-limiting examples of C₆₋₁₂aryl comprise phenyl, biphenylyl, biphenylenyl, or 1-or 2-naphthanelyl; 5- or 6-tetralinyl, 1-, 2-, 3-, 4-, 5-, 6-, 7- or 8-azulenyl, 4-, 5-, 6 or 7-indenyl, 4- or 5-indanyl, 5-, 6-, 7- or 8-tetrahydronaphthyl, 1,2,3,4-tetrahydronaphthyl, and 1,4-dihydronaphthyl. Where one or more carbon atom in an aryl group are replaced with a heteroatom, preferably selected from N, O and S, the resultant ring(s) is referred to herein as a heteroaromatic ring(s). Non-limiting examples of a mono-, bi-, or tricyclic heteroaromatic group or ring(s) containing one or more heteroatom(s), preferably selected from N, O and S, include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, pyranyl, carbazolyl, acridinyl, quinolinyl, benzimidazolyl, benzoimidazolyl, benzthiazolyl, purinyl, azapurinyl, cinnolinyl, pterdinyl.

The term “heteroaromatic ring” or “heteroaryl” as used herein, by itself or as part of another group refers but is not limited to 5 to 12 carbon-atom aromatic rings or ring systems containing 1 to 2 rings which are fused together or linked covalently, typically containing 5 to 6 atoms; at least one of which is aromatic in which one or more carbon atoms in one or more of these rings can be replaced by oxygen, nitrogen or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl include: pyridyl, pyrrolyl, quinolinyl, furanyl, thienyl, oxadiazolyl, thiadiazolyl, thiazolyl, oxazolyl, pyrazolyl, triazolyl, imidazolyl, pyrimidinyl, indolyl, pyrazinyl or indazolyl.

The term “halogen” or “halo”, as a group or part of a group is generic for fluoro, chloro, bromo, iodo.

The term “C₁₋₁₀alkyloxy”, as a group or part of a group, refers to a group having the Formula —OR^{a} wherein R^{a} is C₁₋₁₀alkyl as defined herein above. Non-limiting examples of suitable C₁₋₆alkoxy include methoxy, ethoxy, propoxy, isopropoxy, butoxy, isobutoxy, sec-butoxy, tert-butoxy, pentyloxy and hexyloxy.

The term “C₁₋₁₀alkylamino”, as a group or part of a group, refers to a group having the Formula —NH—R^{b} wherein R^{b} is C₁₋₁₀alkyl as defined herein above. Non-limiting examples of suitable C₁₋₆alkylamino include methylamino, ethylamino, propylamino, isopropylamino, butylamino, isobutylamino, sec-butylamino, tert-butylamino, pentylamino, and hexylamino.

The term “C₂₋₁₀alkenylamino”, as a group or part of a group, refers to a group having the Formula —NH—R^{c} wherein R^{c} is C₂₋₁₀alkenyl as defined herein above.

The term “C₂₋₁₀alkynylamino”, as a group or part of a group, refers to a group having the Formula —NH—R^{d} wherein R^{d} is C₂₋₁₀alkynyl as defined herein above.

As used herein, the term “functional groups” means in the case of unprotected: hydroxy-, thiolo-, aminofunction, carboxylic acid and in the case of protected: C₁₋₆alkoxy, N—, O—, S— acetyl, carboxylic acid ester.

In certain embodiments, the p53-activating agent as taught herein may be a compound having the structure of Formula II, or a pharmaceutically acceptable salt or prodrug thereof,

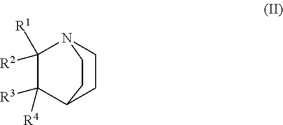

wherein

R¹ and R² are independently selected from methoxymethyl, hydroxymethyl, hydrogen, or a methylene group linked to the nitrogen atom of an amine-substituted phenyl group, to a nitrogen atom contained in the ring structure of a purine, 8-azapurine, or benzimidazol residue, or R¹ and R² may together represent a double bonded methylene group, and;

R³ and R⁴ may together represent an oxygen atom being double bonded, or R³ and R⁴ are independently selected from hydrogen, hydroxyl, and benzoyloxy, with the proviso that when either of R³ and R⁴ is a benzoyloxy group, both R¹ and R² are hydrogen.

In certain embodiments, the p53-activating agent as defined herein may be selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, or 2-(2,6-dichloro-9-methylenepurine)-3-quinuclidinone, or a pharmaceutically acceptable salt thereof.

The term “PRIMA-1^{Met}”, as used herein, refers to a compound of Formula Ia, or a pharmaceutically acceptable salt thereof.

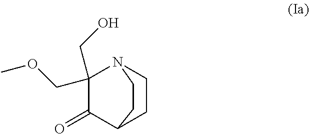

The terms “PRIMA-1^{Met}”, “2-hydroxymethyl-2-methoxymethylazabicyclo[2.2.2]octan-3-one”, “Prima-1^{Met}”, “PRI MA-1MET”, “APR-246”, “5291-32-7”, “Prima 1MET”, “SureCN2228161”, “UNII-Z41TGB4080”, “CTK8G2576”, “AG-F-80925”, and “NCGC00346881-01” may be used interchangeably herein.

The term “PRIMA-1”, as used herein, refers to a compound of Formula Ib, or a pharmaceutically acceptable salt thereof.

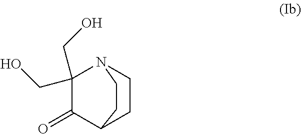 [SMILES: O=C1C2CCN(CC2)C1(CO)CO]

The terms “PRIMA-1”, “2,2-bis(hydroxymethyl)-1-azabicyclo[2,2,2]octan-3-one”, “Prima-1”, “NSC281668”, “NSC-281668”, “MLS003115529”, “5608-24-2”, and “p53 Reactivation and Induction of Massive Apoptosis” may be used interchangeably herein.

The term “PRIMA-2”, as used herein, refer to a compound of Formula Ic, or a pharmaceutically acceptable salt thereof. The terms “PRIMA-2”, “9-(azabicyclo[2.2.2]octan-3-one)-6-chloro-9H-purine”, and “Prima-2” may be used interchangeably herein.

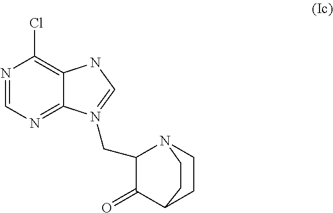

The term “PRIMA-3”, as used herein, refers to a compound of Formula Id, or a pharmaceutically acceptable salt thereof. The terms “PRIMA-3”, “2-(hydroxymethyl)quinuclidine-3,3-diol”, and “Prima-3” may be used interchangeably herein.

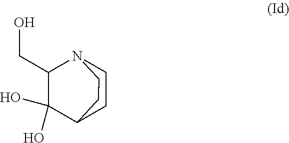 [SMILES: OCC1N2CCC(CC2)C1(O)O]

In certain embodiments, the p53-activating agent as defined herein agent may be selected from the group consisting of 2-hydroxymethyl-2-methoxymethylazabicyclo[2.2.2]octan-3-one (PRIMA-1^{Met}), 2,2-bis(hydroxymethyl)-1-azabicyclo[2,2,2]octan-3-one (PRIMA-1), 9-(azabicyclo[2.2.2]octan-3-one)-6-chloro-9H-purine (PRIMA-2), and 2-(hydroxymethyl)quinuclidine-3,3-diol (PRIMA-3).

In certain embodiments, the p53-activating agent as defined herein agent may be 2-hydroxymethyl-2-methoxymethylazabicyclo[2.2.2]octan-3-one (PRIMA-1^{Met}).

In certain embodiments, the p53-activating agent as defined herein may be selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, or 2-(2,6-dichloro-9-methylenepurine)-3-quinuclidinone, or a pharmaceutically acceptable salt thereof; preferably the p53-activating agent as defined herein agent may be selected from the group consisting of 2-hydroxymethyl-2-methoxymethylazabicyclo[2.2.2]octan-3-one (PRI MA-1^{Met}), 2,2-bis(hydroxymethyl)-1-azabicyclo[2,2,2]octan-3-one (PRIMA-1), 9-(azabicyclo[2.2.2]octan-3-one)-6-chloro-9H-purine (PRIMA-2), and 2-(hydroxymethyl)quinuclidine-3,3-diol (PRIMA-3); more preferably the p53-activating agent as defined herein agent may be 2-hydroxymethyl-2-methoxymethylazabicyclo[2.2.2]octan-3-one (PRIMA-1^{Met}), and said BRAF-inhibiting agent as defined herein may be selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihyd ro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine; preferably said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib) or N-{3-[5-(2-aminopyrimidin-4-yl)-2-tert-butyl-1,3-thiazol-4-yl]-2-fluorophenyl}-2,6-difluorobenzenesulfonamide (dabrafenib); more preferably said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib).

In certain embodiments, the p53-activating agent as defined herein may be selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, or 2-(2,6-dichloro-9-methylenepurine)-3-quinuclidinone, or a pharmaceutically acceptable salt thereof, and said BRAF-inhibiting agent as defined herein may be selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine.

In certain embodiments, the p53-activating agent as defined herein agent may be selected from the group consisting of 2-hydroxymethyl-2-methoxymethylazabicyclo[2.2.2]octan-3-one (PRIMA-1^{Met}), 2,2-bis(hydroxymethyl)-1-azabicyclo[2,2,2]octan-3-one (PRIMA-1), 9-(azabicyclo[2.2.2]octan-3-one)-6-chloro-9H-purine (PRIMA-2), and 2-(hydroxymethyl)quinuclidine-3,3-diol (PRIMA-3), and said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib) or N-{3-[5-(2-aminopyrimidin-4-yl)-2-tert-butyl-1,3-thiazol-4-yl]-2-fluorophenyl}-2,6-difluorobenzenesulfonamide (dabrafenib).

In certain embodiments, said p53-activating agent as defined herein agent may be 2-hydroxymethyl-2-methoxymethylazabicyclo[2.2.2]octan-3-one (PRIMA-1^{Met}), and said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib).

In certain embodiments, said p53-activating agent as defined herein agent may be administered simultaneously or sequentially with said BRAF-inhibiting agent as defined herein and with a MEK-inhibiting agent as defined herein.

In certain embodiments, said p53-activating agent as defined herein may be administered after administration of said BRAF-inhibiting agent as defined herein and said MEK-inhibiting agent as defined herein.

In certain embodiments, said p53-activating agent as defined herein may be administered before administration of said BRAF-inhibiting agent as defined herein and said MEK-inhibiting agent as defined herein.

In certain embodiments, the p53-activating agent as defined herein may be selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, or 2-(2,6-dichloro-9-methylenepurine)-3-quinuclidinone, or a pharmaceutically acceptable salt thereof; preferably the p53-activating agent as defined herein agent may be selected from the group consisting of 2-hydroxymethyl-2-methoxymethylazabicyclo[2.2.2]octan-3-one (PRIMA-1^{Met}), 2,2-bis(hydroxymethyl)-1-azabicyclo[2,2,2]octan-3-one (PRIMA-1), 9-(azabicyclo[2.2.2]octan-3-one)-6-chloro-9H-purine (PRIMA-2), and 2-(hydroxymethyl)quinuclidine-3,3-diol (PRIMA-3); more preferably the p53-activating agent as defined herein agent may be 2-hydroxymethyl-2-methoxymethylazabicyclo[2.2.2]octan-3-one (PRIMA-1^{Met}), and said BRAF-inhibiting agent as defined herein may be selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine; preferably said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib) or N-{3-[5-(2-aminopyrimidin-4-yl)-2-tert-butyl-1,3-thiazol-4-yl]-2-fluorophenyl}-2,6-difluorobenzenesulfonamide (dabrafenib); more preferably said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib), and said MEK-inhibiting agent may be selected from the group consisting of N-[3-[3-cyclopropyl-5-(2-fluoro-4-iodoanilino)-6,8-dimethyl-2,4,7-trioxopyrido[4,3-d]pyrimidin-1-yl]phenyl]acetamide (trametinib), N-[(2S)-2,3-dihydroxypropyl]-3-(2-fluoro-4-iodoanilino)pyridine-4-carboxamide (pimasertib), 6-(4-bromo-2-chloroanilino)-7-fluoro-N-(2-hydroxyethoxy)-3-methylbenzimidazole-5-carboxamide (selumetinib), 6-(4-bromo-2-fluoroanilino)-7-fluoro-N-(2-hydroxyethoxy)-3-methylbenzimidazole-5-carboxamide (MEK162), N-[(2R)-2,3-dihydroxypropoxy]-3,4-difluoro-2-(2-fluoro-4-iodoanilino)benzamide (PD-325901), [3,4-difluoro-2-(2-fluoro-4-iodoanilino)phenyl]-[3-hydroxy-3-[(2S)-piperidin-2-yl]azetidin-1-yl]methanone (cobimetenib), and 2-(2-chloro-4-iodoanilino)-N-(cyclopropylmethoxy)-3,4-difluorobenzamide (CI-1040); preferably said MEK-inhibiting agent may be N-[3-[3-cyclopropyl-5-(2-fluoro-4-iodoan ilino)-6,8-d imethyl-2,4,7-trioxopyrido[4,3-d]pyrimidin-1-yl]phenyl]acetamide (trametinib) or N-[(2S)-2,3-dihydroxypropyl]-3-(2-fluoro-4-iodoanilino)pyridine-4-carboxamide (pimasertib).

In certain embodiments, the p53-activating agent as defined herein may be selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, or 2-(2,6-dichloro-9-methylenepurine)-3-quinuclidinone, or a pharmaceutically acceptable salt thereof, and said BRAF-inhibiting agent as defined herein may be selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine, and said MEK-inhibiting agent may be selected from the group consisting of N-[4-[3-cyclopropyl-5-(2-fluoro-4-iodoanilino)-6,8-dimethyl-2,4,7-trioxopyrido[4,3-d]pyrimidin-1-yl]phenyl]acetamide (trametinib), N-[(2S)-2,3-dihydroxypropyl]-3-(2-fluoro-4-iodoanilino)pyridine-4-carboxamide (pimasertib), 6-(4-bromo-2-chloroanilino)-7-fluoro-N-(2-hydroxyethoxy)-3-methylbenzimidazole-5-carboxamide (selumetinib), 6-(4-bromo-2-fluoroanilino)-7-fluoro-N-(2-hydroxyethoxy)-3-methylbenzimidazole-5-carboxamide (MEK162), N-[(2R)-2,3-dihydroxypropoxy]-3,4-difluoro-2-(2-fluoro-4-iodoanilino)benzamide (PD-325901), [3,4-difluoro-2-(2-fluoro-4-iodoanilino)phenyl]-[3-hydroxy-3-[(2S)-piperidin-2-yl]azetidin-1-yl]methanone (cobimetenib), and 2-(2-chloro-4-iodoan ilino)-N-(cyclopropyl methoxy)-3,4-difluorobenzamide (CI-1040).

In certain embodiments, the p53-activating agent as defined herein agent may be selected from the group consisting of 2-hydroxymethyl-2-methoxymethylazabicyclo[2.2.2]octan-3-one (PRIMA-1^{Met}), 2,2-bis(hydroxymethyl)-1-azabicyclo[2,2,2]octan-3-one (PRIMA-1), 9-(azabicyclo[2.2.2]octan-3-one)-6-chloro-9H-purine (PRIMA-2), and 2-(hydroxymethyl)quinuclidine-3,3-diol (PRIMA-3), and said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib) or N-{3-[5-(2-aminopyrimidin-4-yl)-2-tert-butyl-1,3-thiazol-4-yl]-2-fluorophenyl}-2,6-difluorobenzenesulfonamide (dabrafenib), and said MEK-inhibiting agent may be N-[3-[3-cyclopropyl-5-(2-fluoro-4-iodoanilino)-6,8-dimethyl-2,4,7-trioxopyrido[4,3-d]pyrimidin-1-yl]phenyl]acetamide (trametinib) or N-[(2S)-2,3-dihydroxypropyl]-3-(2-fluoro-4-iodoanilino)pyridine-4-carboxamide (pimasertib).

In certain embodiments, said p53-activating agent as defined herein agent may be 2-hydroxymethyl-2-methoxymethylazabicyclo[2.2.2]octan-3-one (PRIMA-1^{Met}), and said BRAF-inhibiting agent as defined herein may be N-(3-{[5-(4-chlorophenyl)-1H-pyrrolo[2,3-b]pyridin-3-yl]carbonyl}-2,4-difluorophenyl) propane-1-sulfonamide (vemurafenib), and said MEK-inhibiting agent may be N-[3-[3-cyclopropyl-5-(2-fluoro-4-iodoanilino)-6,8-dimethyl-2,4,7-trioxopyrido[4,3-d]pyrimidin-1-yl]phenyl]acetamide (trametinib) or N-[(2S)-2,3-dihydroxypropyl]-3-(2-fluoro-4-iodoan ilino)pyridine-4-carboxamide (pimasertib).

As used herein, the term “melanoma” refers to cancer of melanocytes or a malignant tumor of melanocytes.

The term “melanocytes” refers to cells which produce dark pigment, melanin, which is responsible for the colour of skin. Melanocytes predominantly occur in skin, but are also found in other parts of the body, including the bowel and the eye. Melanoma may originate in any part of the body that contains melanocytes.

As used herein, the term “cancer” refers to a malignant neoplasm characterized by deregulated or unregulated cell growth.

As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function.

Tumors, tumor tissue and tumor cells may be benign, pre-malignant or malignant, or may represent a lesion without any cancerous potential. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignant” refers to a non-benign tumor.

Generally, melanoma may be classified in one of the following stages:

Stage 0: This refers to melanoma in situ, which means melanoma cells are found only in the epidermis (the outer layer of skin). This stage of melanoma has virtually no metastatic potential, which means it is very unlikely that it will spread to other parts of the body.

Stage I: The melanoma is still only in the skin and is very thin. Stage IA melanoma is 1.0 mm or thinner and has no ulceration. Stage IB may describe a melanoma that is the same thickness but has ulceration or a melanoma that is slightly thicker (between 1.1 mm and 2.0 mm) with no ulceration.

Stage II: Stage II melanoma is thicker than stage I melanoma, extending through the epidermis and into the dermis (dense inner layer of the skin), and it has a slightly higher chance of spreading. Stage II is divided into three smaller groups—A, B, or C—depending on how thick the melanoma is and whether or not there is ulceration.

Stage III: This stage describes melanoma that has spread through the lymphatic system (part of the immune system and drains fluid from body tissues through a series of tubes) either to a regional lymph node (lymph nodes near where the cancer started) or to a skin site on the way to a lymph node (“in-transit metastasis”). Stage III is also divided into substages—A, B, or C—depending on the size and number of lymph nodes involved with melanoma.

Stage IV: This stage describes melanoma that has spread through the bloodstream to other, distant parts of the body, such as the lung, liver, brain, bone, or gastrointestinal tract. Stage IV is further divided into M1a (metastasis only to the skin and/or soft tissue sites), M1b (metastasis to the lung), and M1c (metastasis anywhere else).

Recurrent: Recurrent melanoma is melanoma that has come back after treatment. If there is a recurrence, the cancer may need to be staged again (re-staging).

The term “metastatic” or “metastasis” generally refers to the spread of a cancer from one organ or tissue to another non-adjacent organ or tissue.

In certain embodiments, the melanoma may be lentigo maligna, lentigo maligna melanoma, superficial spreading melanoma, acral lentiginous melanoma, mucosal melanoma, nodular melanoma, polypoid melanoma, desmoplastic melanoma, amelanotic melanoma, or soft-tissue melanoma.

Term “lentigo melanoma” generally refers to a melanoma in situ that consists of malignant cells but does not show invasive growth. The terms “lentigo melanoma” and “lentiginous melanoma on sun-damaged skin” may be used interchangeably.

The term “lentigo maligna melanoma” generally refers to a melanoma that has evolved from a lentigo maligna.

The term “superficial spreading melanoma”, “superficially spreading melanoma”, or “SSM” generally refers to cutaneous melanoma in Caucasians. Often, SSM evolves from a precursor lesion, usually a dysplastic nevus. Otherwise it arises in previously normal skin. The microscopic hallmarks may include: large melanocytic cells with nest formation along the dermo-epidermal junction; invasion of the upper epidermis in a pagetoid fashion (discohesive single cell growth); the pattern of rete ridges is often effaced; invasion of the dermis by atypical, pleomorphic melanocytes; absence of the maturation typical of nevus cells; and mitoses.

The term “acral lentiginous melanoma” generally refers to a kind of lentiginous skin melanoma. Typical signs of acral lentiginous melanoma may include longitudinal tan, black, or brown streak on a finger or toe nail (melanonychia striata), pigmentation of proximal nail fold, and areas of dark pigmentation on palms of hands or soles of feet.

The term “mucosal melanoma” refers to a rare cutaneous condition characterized by a melanoma of the mucous membranes.

The term “nodular melanoma” refers to an aggressive form of melanoma which tends to grow more rapidly in thickness (and penetrate the skin) than in diameter. The microscopic hallmarks may include dome-shaped at low power; epidermis thin or normal; dermal nodule of melanocytes with a pushing growth pattern; and no radial growth phase.

The term “polypoid melanoma” refers to a rare cutaneous condition, a virulent variant of nodular melanoma.

The term “desmoplastic melanoma” refers to a rare cutaneous condition characterized by a deeply infiltrating type of melanoma with an abundance of fibrous matrix. The terms “desmoplastic melanoma”, “Neurotropic melanoma”, “Spindled melanoma” may be used interchangeably herein.

The term “amelanotic melanoma” refers to a type of skin cancer in which the cells do not make melanin.

The term “soft-tissue melanoma” or “clear-cell sarcoma” (formerly known as malignant melanoma of the soft parts) refers to a rare form of cancer called sarcoma. Sarcoma is known to occur mainly in the soft tissues and dermis.

In certain embodiments, the melanoma may comprise a cell comprising expression of BRAF comprising an activating mutation.

In certain embodiments, the melanoma may comprise a cell comprising expression of ^{V600E}BRAF. In certain embodiments, the melanoma may comprise a cell comprising expression of ^{V600K}BRAF.

In certain embodiments, the melanoma may comprise a cell with instrinsic or acquired resistance to the BRAF-inhibiting agent.

In addition, the invention provides a pharmaceutical composition which comprises a therapeutically effective amount of the agents or compounds as defined herein, or pharmaceutically acceptable salts thereof, and a pharmaceutically acceptable carrier and/or additive selected from the group of: fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutically acceptable” as used herein is consistent with the meaning of said term in the art and means compatible with the other ingredients of a pharmaceutical composition and not deleterious to the recipient thereof.

The term “pharmaceutically acceptable salts” as used herein means an inorganic acid addition salt such as hydrochloride, sulfate, and phosphate, or an organic acid addition salt such as acetate, maleate, fumarate, tartrate, and citrate. Examples of pharmaceutically acceptable metal salts are alkali metal salts such as sodium salt and potassium salt, alkaline earth metal salts such as magnesium salt and calcium salt, aluminum salt, and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine, and phenylalanine.

The pharmaceutical composition can be prepared in a manner known per se to one of skill in the art. For this purpose, at least one compound according to the invention or a cyclodextrin salt thereof as defined above, one or more solid or liquid pharmaceutical excipients and, if desired, in combination with other pharmaceutical active compounds, are brought into a suitable administration form or dosage form which can then be used as a pharmaceutical in human medicine or veterinary medicine.

In certain embodiments, the invention provides a p53-activating agent as taught herein, for use in the treatment of melanoma, such as preferably but without limitation melanoma comprising a cell comprising expression of ^{V600E/K}BRAF, wherein said p53-activating agent is administered simultaneously or sequentially with a BRAF-inhibiting agent as taught herein.

Also provided according to the present invention is the use of a p53-activating agent as taught herein for the manufacture of a medicament for the treatment of melanoma, such as preferably but without limitation melanoma comprising a cell comprising expression of ^{V600E/K}BRAF, wherein said p53-activating agent is administered simultaneously or sequentially with a BRAF-inhibiting agent as taught herein.

Also provided according to the present invention is a method for treating melanoma, such as preferably but without limitation melanoma comprising a cell comprising expression of ^{V600E/K}BRAF, in a subject in need of such treatment, comprising administering to said subject a therapeutically or prophylactically effective amount of a p53-activating agent as taught herein, wherein said p53-activating agent is administered simultaneously or sequentially with a BRAF-inhibiting agent as taught herein.

In certain embodiments, the p53-activating agent may be administered simultaneously with the BRAF-inhibiting agent. In certain embodiments, the p53-activating agent and the BRAF-inhibiting agent may be administered simultaneously.

In certain embodiments, the p53-activating agent may be administered after administration of the BRAF-inhibiting agent. In certain embodiments, the p53-activating agent may be administered before administration of the BRAF-inhibiting agent. In certain embodiments, the p53-activating agent and the BRAF-inhibiting agent may be administered sequentially in any order, for example, the p53-activating agent can be administered and subsequently the BRAF-inhibiting agent can be administered, or the BRAF-inhibiting agent can be administered and subsequently the p53-activating agent can be administered. The time frame between both administration steps can in such case be anywhere between 4 hours and 4 days, such as between 12 hours and 2 days, or between 12 and 36 hours.

In certain embodiments, the p53-activating agent and the BRAF-inhibiting agent may be administered separately, i.e. by different routes of administration, or may be administered together, i.e. by the same route of administration. In certain embodiments, the p53-activating agent and the BRAF-inhibiting agent may be administered simultaneously but separately, i.e. by different routes of administration.

In certain embodiments, the p53-activating agent can be administered by parenteral administration such as by intraperitoneal (IP) administration, in particular by IP injection. In certain embodiments, the BRAF-inhibiting agent can be administered by parenteral administration, such as by intraperitoneal (IP) administration, in particular by IP injection. Preferably, the subject in who said administration is to be performed may be human.

In certain embodiments, the subject may be a subject with a melanoma responsive to treatment with a p53-activating agent as defined herein in combination with a BRAF-inhibiting agent as defined herein, as determined by any one of the methods as taught herein for predicting responsiveness of melanoma to treatment with the p53-activating agent in combination with the BRAF-inhibiting agent in a subject.

In some embodiments, the p53-activating agent and the BRAF-inhibiting agent may be comprised in a composition or formulation. In some embodiments, the p53-activating agent and the BRAF-inhibiting agent may be comprised in a pharmaceutical composition or pharmaceutical formulation. Accordingly, a further aspect relates to a composition or formulation as taught herein, for use as a medicament, preferably for use in the treatment of melanoma, such as preferably but without limitation melanoma comprising a cell comprising expression of ^{V600E/K}BRAF . In particular, certain embodiments provide a composition or formulation comprising an p53-activating agent as taught herein and a BRAF-inhibiting agent, for use as a medicament, preferably for use in the treatment of melanoma, such as preferably but without limitation melanoma comprising a cell comprising expression of ^{V600E/K}BRAF.

The use of a p53-activating agent and a BRAF-inhibiting agent in the treatment of melanoma, such as preferably but without limitation melanoma comprising a cell comprising expression of ^{V600E/K}BRAF, is advantageous since, as shown in the examples, a p53-activating agent is able to break both intrinsic and acquired resistance of the melanoma to the BRAF-inhibiting agent, thereby increasing the survival rate of subjects having melanoma.

Also provided according to the present invention is the use of a p53-activating agent as taught herein and a BRAF-inhibiting agent as taught herein for the manufacture of a medicament for the treatment of melanoma, such as preferably but without limitation melanoma comprising a cell comprising expression of ^{V600E/K}BRAF.

In some embodiments, the p53-activating agent and the BRAF-inhibiting agent may be comprised in a kit of parts, preferably in a pharmaceutical kit of parts. Accordingly, a further aspect relates to a kit of parts as taught herein for the manufacture of a medicament for the treatment of melanoma, such as preferably but without limitation melanoma comprising a cell comprising expression of ^{V600E/K}BRAF . In particular, certain embodiments provide the use of a kit of parts comprising a p53-activating agent as taught herein and a BRAF-inhibiting agent as taught herein, for the manufacture of a medicament for the treatment of melanoma, such as preferably but without limitation melanoma comprising a cell comprising expression of ^{V600E/K}BRAF.

In some embodiments, the p53-activating agent and the BRAF-inhibiting agent may be comprised in a composition or formulation, preferably in a pharmaceutical composition or pharmaceutical formulation. Accordingly, a further aspect relates to a composition or formulation as taught herein for the manufacture of a medicament for the treatment of melanoma, such as preferably but without limitation melanoma comprising a cell comprising expression of ^{V600E/K}BRAF . In particular, certain embodiments provide the use of a composition or formulation comprising a p53-activating agent and a BRAF-inhibiting agent, for the manufacture of a medicament for the treatment of melanoma, such as preferably but without limitation melanoma comprising a cell comprising expression of ^{V600E/K}BRAF.

Also provided according to the present invention is a method for treating melanoma, such as preferably but without limitation melanoma comprising a cell comprising expression of ^{V600E/K}BRAF, in a subject in need of such treatment, comprising administering to said subject a therapeutically or prophylactically effective amount of a p53-activating agent as taught herein and of a BRAF-inhibiting agent as taught herein.

In some embodiments, the p53-activating agent and the BRAF-inhibiting agent may be comprised in a kit of parts, preferably in a pharmaceutical kit of parts. Accordingly, a further aspect relates to a method for treating melanoma, such as preferably but without limitation melanoma comprising a cell comprising expression of ^{V600E/K}BRAF, in a subject in need of such treatment, comprising administering to said subject a kit of parts as taught herein. In particular, certain embodiments provide a method for treating melanoma, such as preferably but without limitation melanoma comprising a cell comprising expression of ^{V600E/K}BRAF, in a subject in need of such treatment, comprising administering to said subject a therapeutically or prophylactically effective amount of a kit of parts comprising a p53-activating agent as taught herein and a BRAF-inhibiting agent as taught herein.

In some embodiments, the p53-activating agent and the BRAF-inhibiting agent may be comprised in a composition or formulation, preferably in a pharmaceutical composition or pharmaceutical formulation. Accordingly, a further aspect relates to a method for treating melanoma, such as preferably but without limitation melanoma comprising a cell comprising expression of ^{V600E/K}BRAF , in a subject in need of such treatment, comprising administering to said subject a composition or formulation as taught herein. In particular, certain embodiments provide a method for treating melanoma, such as preferably but without limitation melanoma comprising a cell comprising expression of ^{V600E/K}BRAF, in a subject in need of such treatment, comprising administering to said subject a therapeutically or prophylactically effective amount of a composition or formulation comprising a p53-activating agent as taught herein and a BRAF-inhibiting agent as taught herein.

In a further embodiment, the invention provides for a method of treating melanoma in a patient needing such therapy, comprising administering a therapeutically effective amount of agents, compounds, pharmaceutical compositions or kits as defined herein to a patient in need thereof.

The terms “treat”, “treating”, or “treatment” as used herein includes treating any one or more of the conditions underlying or characteristic of cancer. Treatment of cancer means administration of a medicament in the form of agents, compounds, pharmaceutical compositions or kits as defined herein with the result that cancer is stabilized, reduced or the patient is cured. As used herein, the terms “treat”, “treating”, or “treatment” can furthermore refer to both therapeutic treatment and prophylactic or preventative measures, wherein the object is to prevent or slow down (lessen) an undesired physiological change or disorder, such as the development or spread of proliferative disease, e.g., cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

Except when noted, “subject” or “patient” are used interchangeably and refer to animals, preferably vertebrates, more preferably mammals, and specifically includes human patients and non-human mammals. “Mammalian” subjects include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orang-utans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Accordingly, “subject” or “patient” as used herein means any mammalian patient or subject to which the compositions of the invention can be administered. Preferred patients are human subjects.

As used herein, a phrase such as “a subject in need of treatment” includes subjects, such as mammalian subjects, that would benefit from treatment of a given condition, preferably a proliferative disease, such as, e.g., cancer, e.g. melanoma as defined herein.

The term “therapeutically effective amount” refers to an amount of the agents, compounds, pharmaceutical compositions or kits as defined herein effective to treat melanoma in a subject, i.e., to obtain a desired local or systemic effect and performance.

By means of example and not limitation, in the case of proliferative disease such as melanoma, the therapeutically effective amount of the agents, compounds, pharmaceutical compositions or kits as defined herein may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; enhance efficacy of another cancer therapy; and/or relieve to some extent one or more of the symptoms associated with the cancer. To the extent the agents, compounds, pharmaceutical compositions or kits as defined herein may prevent growth and/or kill existing cancer cells, it may be cytostatic and/or cytotoxic. For cancer therapy, efficacy can, for example, be measured by assessing the time to disease progression (TTP) and/or determining the response rate (RR). The term thus refers to the quantity of the agents, compounds, pharmaceutical compositions or kits as defined herein that elicit(s) the biological or medicinal response in a tissue, system, animal, or human that is being sought by a researcher, veterinarian, medical doctor or other clinician, which includes alleviation of the symptoms of the cancer being treated. In particular, these terms refer to the quantity of the agents, compounds, pharmaceutical compositions or kits as defined herein which is necessary to prevent, cure, ameliorate, or at least minimize the clinical impairment, symptoms, or complications associated with cancer in either a single or multiple doses.

The agents, compounds, pharmaceutical compositions or kits as defined herein may be used alone or in combination with any of the cancer therapies selected from the group comprising chemotherapy, radiation therapy, immunotherapy, and/or gene therapy. As used herein the term “cancer therapy” is meant to encompass radiation therapy, chemotherapy, immunotherapy, gene-based therapy, surgery, as well as combinations thereof.

The agents, compounds, pharmaceutical compositions or kits as defined herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods. By means of non-limiting examples, such a formulation may be in a form suitable for oral administration, for parenteral administration (such as by intravenous, intramuscular, or subcutaneous injection, or intravenous infusion), for topical administration (including ocular), for administration by inhalation, by a skin patch, by an implant, by a suppository, etc. Such suitable administration forms—which may be solid, semi-solid, or liquid, depending on the manner of administration—as well as methods and carriers, diluents and excipients for use in the preparation thereof, will be clear to the skilled person; reference is made to for instance U.S. Pat. No. 6,372,778, U.S. Pat. No. 6,369,086, U.S. Pat. No. 6,369,087, and U.S. Pat. No. 6,372,733, as well as to the standard handbooks, such as the latest edition of Remington's Pharmaceutical Sciences. For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiological sodium chloride solution, alcohols such as ethanol, glycerol, polyols, sucrose, invert sugar, glucose, mannitol, vegetable oils, etc. It is also possible to lyophilize the nucleic acid and/or the active compound and to use the resulting lyophilisates, for example, for preparing preparations for injection or infusion. Suitable carriers for microcapsules, implants or rods are, for example, copolymers of glycolic acid and lactic acid.

The pharmaceutical compositions of this invention can be administered to humans in dosage ranges specific for each compound comprised in said compositions. The compounds comprised in said composition can be administered together or separately.

It will be understood, however, that specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In certain embodiments, the present invention relates to a method for determining resistance of melanoma to a BRAF-inhibiting agent as defined herein in a subject, wherein the method may comprise the steps of:

(i) preparing a cell culture from a melanoma sample obtained from the subject, and

(ii) determining the cytotoxicity of cells of the cell culture to said BRAF-inhibiting agent, wherein the melanoma is intrinsically resistant to said BRAF-inhibiting agent when the IC50 of the cells is at least 10 μM, and wherein the melanoma is sensitive to said BRAF-inhibiting agent when the IC50 of the cells is less than 10 μM.

In certain embodiments, the methods as taught herein for predicting the development of resistance to said BRAF-inhibiting agent in a melanoma initially sensitive to said BRAF-inhibiting agent, may further comprise the steps of:

(iii) treating the melanoma cell culture by chronic exposure with increasing concentrations of said BRAF-inhibiting agent during at least about 4 weeks, and

(iv) determining the cytotoxicity of cells of the cell culture to said BRAF-inhibiting agent after said treatment, wherein the melanoma has acquired resistance to the BRAF-inhibiting agent when the IC50 of the cells is at least 10 μM.

In certain embodiments, the melanoma cell culture may be treated by chronic exposure with increasing concentrations of said BRAF-inhibiting agent during at least about four weeks or at least about one month, for instance during at least about two months, at least about four months, at least about six months, at least about eight months, at least about ten months, at least about one year, at least about two years, or at least about three years.

In certain embodiments, the melanoma sample obtained from the subject may originate from a metastasis of the subject, e.g. from skin, lymph node, mucosa, liver, or gastrointestinal tract.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLES

Material and Methods

Effectors

The ^{V600E}BRAF inhibitor vemurafenib and the PI3K inhibitor LY294002 were from Selleck Chemicals (Houston, Tex., USA). The PI3K/mTOR dual inhibitor PF-04691502 and the p53 activator PRIMA-1^{Met} were from Tocris Bioscience (Bristol, UK). They were dissolved, according to the manufacturer's recommendations, in DMSO (vemurafenib, LY294002 and PF-04691502) or water (PRIMA-1^{Met}) at 10⁻² M, aliquoted and stored at −20° C.Melanoma cell lines

A panel of nine human melanoma cell lines, derived from different metastatic sites, were all established in the “Laboratoire d'Oncologie et de Chirurgie Experimentale”, Universite Libre de Bruxelles (Krayem M et al., 2014, Eur J Cancer Oxf Engl 1990, Vol. 50(7):1310-1320 ; Gembarska A et al., 2012 Nat Med.Vol. 18(8):1239-1247). The BRAF, NRAS, TP53 and PTEN mutation status have been evaluated with the next-generation DNA sequencing for 48 genes from cancer panel (TruSeq Amplicon—Cancer Panel, Illumina, San Diego, Calif., USA) and summarized in Table 1.

Cell Culture Conditions

Cells were grown in HAM-F10 medium supplemented with 5% heat-inactivated foetal calf serum, 5% heat-inactivated new-born calf serum and with L-glutamine, penicillin and streptomycin at standard concentrations (all from Gibco, Invitrogen, UK) (culture medium) at 37° C. in a humidified 95% air and 5% CO2 atmosphere. For routine maintenance, cells were propagated in flasks, harvested by trypsinization (0.05% trypsin-EDTA) (Gibco) and subcultured twice weekly. Cells were counted using a TC10™ Automated Cell Counter (Bio-Rad, Hercules, Calif., USA). All cell lines are regularly checked for mycoplasma contamination using MycoAlert® Mycoplasma Detection Kit (Lonza, Rockland, Me., USA).

Cell Proliferation Assay

Cell proliferation was assessed by crystal violet assay (Krayem M et al., 2014, Eur J Cancer Oxf Engl 1990, Vol. 50(7):1310-1320). All cells were seeded in 96-well plates (8×10³ cells/well) in complete medium (day-1). One day after plating (day 0), the culture medium was replaced by fresh medium containing effectors or inhibitors or not depending on experimental conditions, and cells were cultured for 1 additional day (day 1) or 3 additional days (day 3). Culture medium was removed and cells were gently rinsed with phosphate-buffered saline (PBS), fixed with 1% glutaraldehyde/PBS for 15 minutes and stained with 0.1% crystal violet (w/v in water) for 30 minutes. Cells were distained under running tap water and subsequently lysed with 0.2% Triton X-100 for 90 minutes (v/v in water). The absorbance was measured at 570 nm using a Multiskan EX Microplate Photometer (Thermo Scientific, Courtaboeuf Cedex, France). On each plate, blank wells containing medium alone were used to estimate background.

Apoptosis Determination

Apoptotic cell was determined by using Annexin V: PE Apoptosis Detection Kit I (BD Pharmingen, Erembodegem, Belgium), according to the manufacturer's recommendations. Cells were seeded in 6-well plates (2×10⁵ cells/well) in culture medium. One day after plating, the culture medium was replaced by fresh medium containing or not effectors and cells were further incubated for 2 days. Then, culture medium was collected and cells were harvested by trypsinization and mixed with the culture medium collection. Cells were pelleted by brief centrifugation (200 g, 5 minutes) and suspended in 100 μl 1× Binding Buffer (BD Pharmingen). After addition of 5 μl annexin V-FITC and 5 μl 7-amino-actinomycin (7-AAD), cell suspensions were incubated for 15 minutes at room temperature and in the dark. Finally, cells were diluted with 400 μl 1× Binding Buffer and analyzed with a flow cytometrer (FACS Calibur, Becton Dickinson, Franklin Lakes, N.J., USA) within 1 hour.

Western Blot Analysis

Cells were plated in Petri dishes (3×10⁶ cells/dish) in culture medium. One day after plating, the culture medium was replaced by a fresh one and further left for 2 days. Then, cells were exposed or not to effectors for 30 minutes or 24 hours. Cells were lysed using a detergent cocktail (M-PER Mammalian Extraction Buffer) supplemented with protease inhibitors (Halt Protease Inhibitor Cocktail) and phosphatase inhibitors (Halt Phosphatase Inhibitor Cocktail) (all from Pierce, Rockford, Ill., USA). Protein concentrations were determined by the BCA Protein Assay (Pierce) using bovine serum albumin as the standard. Equal amounts of extracted proteins (35 μg) were subjected to 10 or 12% SDS-PAGE and electrotransferred onto nitrocellulose membranes using iBlot® Dry Blotting System (Invitrogen, Life Technologies, Gent, Belgium). Immunodetections were performed using antibodies raised against ^{V600E}BRAF (VE1, 1/1000) (from Spring Bioscience, Pleasanton, Calif., USA), pBRAF (Ser 445) (1/1000), pCRAF (Ser 338) (1/1000), CRAF (1/1000), p110α (C73F8, 1/1000), PTEN (138G6, 1/1000), pAKT (Ser 473) (D9E, 1/500), AKT (40D4, 1/1000), p21 (12D1, 1/1000) and p53 Ser15 (16G8) (all from Cell Signaling Technology, Danvers, Mass., USA), BRAF (F-7, 1/200), pERK (Tyr 204) (E-4, 1/1000), ERK2 (C-14, 1/2000), p53 (DO-1, 1/200) and MDM2 (SMP14, 1/200) (all from Santa Cruz Biotechnology, Santa Cruz, Calif., USA), and MDM4 (8C6, 1/1000), □-actin (C4, 1/5000) (from Millipore, Temecula, Calif., USA). Peroxidase-labeled anti-rabbit IgG antibody (1/5000) or peroxidase-labeled anti-mouse IgG antibody (1/5000) (both from Amersham Pharmacia Biotech, Roosendaal, The Netherlands) were used as secondary reagents to detect corresponding primary antibodies. Bound peroxidase activity was revealed using the SuperSignal® West Pico Chemiluminescent Substrate (Pierce) following the manufacturer's indications. Immunostaining signals were digitalized with a PC-driven LAS-3000 CCD camera (Fujifilm, Tokyo, Japan), using a software specifically designed for image acquisition (Image Reader, Raytest®, Straubenhardt, Germany). Immunoreactive band intensities were quantified using the software AIDA® Image Analyser 3.45 (Raytest®).

Human Melanoma Xenografts

Five to six week old female nude (nu/nu) mice weighing 17-21 g were purchased from Charles River Laboratories (Saint Aubin lès Elbeuf, France). Mice were injected subcutaneously (right and left flank) with 5×10⁶ MM074 (vemurafenib sensitive), MM043 (with intrinsic resistance) or MM074-R cells (with acquired resistance) in 200 μl of 50% Matrigel (from Trevigen, Gaithersburg, Md.) in saline. When tumors reached 200 mm³, mice were randomized into groups of 8 and daily intraperitoneally injected with vehicle, 45 mg/kg vemurafenib, 50 mg/kg PRIMA-1^{Met} or vemurafenib plus PRIMA-1^{Met}. Tumor size and body weight were measured every two days. Tumor volumes were calculated using the formula (L×W×W)/2 (Ji Z et al., 2013, Clin Cancer Res Off J Am Assoc Cancer Res. 19(16):4383-4391), in which L is the length and W is the width as measured with a vernier calliper. The experiments were performed in accordance with the European Union Guidelines and validated by the local Animal Ethics Evaluation Committee (CEBEA protocol: 500N).

Statistical Analysis

The IC50 and IC10 values represent the inhibitory concentrations producing, respectively, 50% and 10% growth reduction and were calculated from dose response curves using GraphPad Prism software (GraphPad Software, La Jolla, Calif., USA). All data are expressed as means±SD of at least three independent experiments; statistical significance was measured by Student's t-test (*p<0.05, **p<0.01, ***p<0.001) using GraphPad Prism software. Differences in tumor volumes and body weight among groups of treated mice were tested using two-way ANOVA; values are presented as means±SEM.

Combination Index Calculation

The synergistic effect was analyzed by the multiple drug-effect equation and quantified by the combination index (CI) using CalcuSyn software version 2.1 (Biosoft, UK). CI values between 0.9 and 1.1 indicates an additive effect; values between 0.7 and 0.9, a moderate synergism; values lower than 0.7, a strong synergism; and antagonism is represented by CI values higher than 1.1.

### Example 1

### Effect of Vemurafenib on ^{V600E}BRAF Melanoma Cells Lines Sensitive to Vemurafenib or ^{V600E}BRAF Melanoma Cells Lines with Intrinsic Resistance to Vemurafenib

The present inventors have established more than 100 melanoma cell lines from human melanoma metastases. More than 20 lines were characterized inter alia with regard to proliferation rate, key mRNA expression, key protein expression and/or activity, BRAF/NRAS/cKIT/MC1R/p53 mutations, and response to various inhibitors/effectors.

The effect of vemurafenib on a panel of nine ^{V600E/K}BRAF melanoma cell lines was evaluated. Six cell lines were found to be sensitive (1050≦2 μm) and five cell lines were found to be resistant (1050>10 μM) to vemurafenib (Sondergaard J N et al., 2010, J Transl Med. 8(1):39; Tap W D et al., 2010, Neoplasia N Y N. 12(8):637-649), as shown in Table 1.

TABLE 1
Description of nine mutant BRAF melanoma cell lines by metastasis site of which
they were derived, melanoma type, BRAF mutation, NRAS mutation, and sensitivity to
vemurafenib (IC50)

| | | | | | | | | IC50 (μM) | |
|---|---|---|---|---|---|---|---|---|---|
| | | | BRAF | NRAS | TP53 | PTEN | | vemurafenib | |
| | Metastatic | Melanoma | Mutation | Mutation | Mutation | Mutation | IC50 (μM) | combined to | |
| Cell line | site (a) | type (b) | status (c) | status (c) | status (c) | status (c) | vemurafenib | PRIMA-1^{Met} (d) | CI (e) |
| MM070 | LN | SSM | V600E | WT | WT | WT | 0.05 | — | — |
| MM034 | LN | unk | V600E | WT | WT | WT | 0.1 | — | — |
| MM050 | LN | SSM | V600E | WT | WT | WT | 0.1 | — | — |
| MM074 | LN | SSM | V600E | WT | WT | WT | 0.1 | 0.05 | 0.73 |
| MM133 | LN | LMM | V600K | WT | WT | WT | 2 | 0.1 | 0.14 |
| MM032 | SK | SSM | V600E | WT | WT | WT | 2 | 0.2 | 0.20 |
| MM043 | Intestine | SSM | V600E | WT | WT | WT | 20 | 0.05 | 0.05 |
| MM074-R | LN | SSM | V600E | WT | WT | WT | 20 | 2 | 0.05 |
| MM029 | LN | SSM | V600K | WT | WT | WT | 30 | 2 | 0.09 |
| MM054 | SK | NM | V600E | WT | WT | WT | 40 | 3 | 0.35 |

(a) LN: lymph node; SK: skin/cutaneous metastasis.
(b) Type of primary melanoma: SSM: superficial spreading melanoma; NM: nodular melanoma; LMM: lentigo maligna melanoma; unk: unknown primary.
(c) WT: wild-type
(d) PRIMA-1^{Met} IC10 values were used to perform experiments evaluating IC50 for vemurafenib/PRIMA-1^{Met} combination.
(e) CI: combination index calculated using CalcuSyn software; CI <0.7 indicates strong synergy.

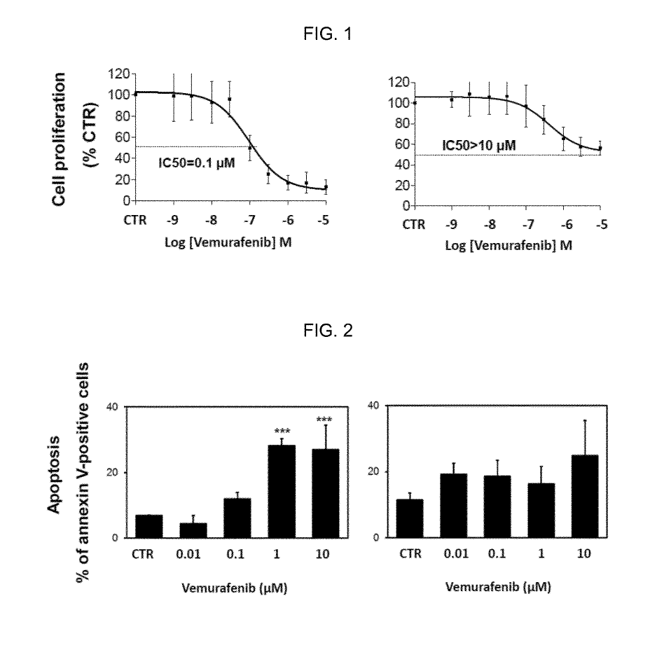

The results of the cell proliferation of two melanoma cell lines, i.e., a cell line sensitive to vemurafenib (MM074) and a cell line resistant to vemurafenib (MM043), are shown in FIG. 1. Apoptosis of the cell line sensitive to vemurafenib (MM074) and tho cell line resistant to vemurafenib (MM043) was determined by using Annexin V : PE Apoptosis Detection Kit I, as described herein. The results are given in FIG. 2.

These data clearly indicate that vemurafenib induces apoptosis in sensitive cells (IC50<10 μM) while no apoptosis is detected in resistant ones (IC50≧10 μM) (FIGS. 1 and 2). Thus, the mechanism of sensitivity of melanoma cells to vemurafenib may be associated with the induction of apoptosis.

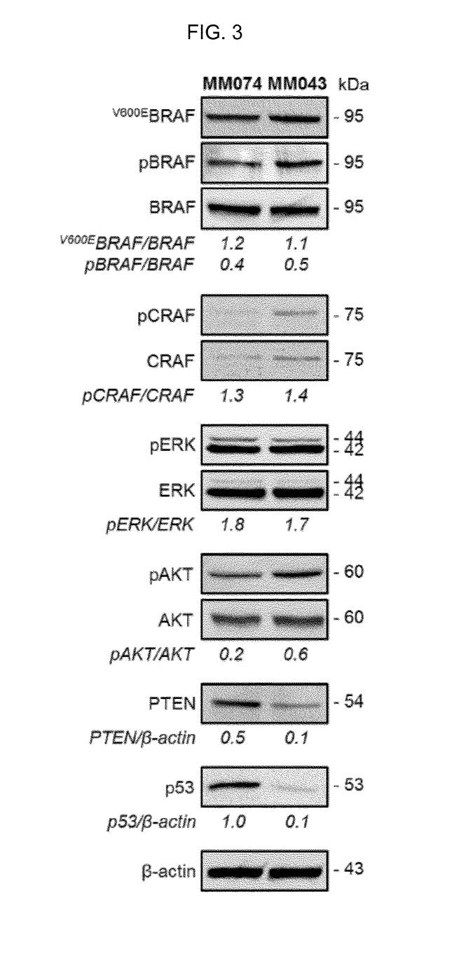

The present inventors found, by comparing various key pathway effectors between the cell line sensitive to vemurafenib (MM074) and the cell line resistant to vemurafenib (MM043), that intrinsic resistance is associated with high AKT phosphorylation, low PTEN, and low p53 expression, while similar inhibitions of ERK phosphorylation were achieved in both lines (FIG. 3A, left panels: MM074; right panels: MM043).

### Example 2

### Effect of Combination of Vemurafenib and the p53 Activator PRIMA-1^{Met} on ^{V600E}BRAF Melanoma Cells Lines Sensitive to Vemurafenib or ^{6V00E}BRAF Melanoma Cells Lines with Intrinsic Resistance to Vemurafenib

The present inventors realized that PI3K inhibition and/or PTEN upregulation can effectively decrease the phosphorylation of AKT and can potentiate the effect of vemurafenib in resistant cells. One possibility to stimulate PTEN and inhibit PI3K is to restore p53 expression and/or activity (Stambolic et al., 2001, Mol. Cell., 8, 317-325; Astanehe et al., 2008, J. Cell Sci., 121, 664-74). PRIMA-1^{Met} is a drug which increases the transcriptional activity of both mutant and wild type p53 (Bao et al., 2011, Cell Cycle, 10, 301-307). The present inventors have studied the effect of the combination of vemurafenib (Vemu) and PRIMA-1^{Met} on ^{V600E}BRAF melanoma cells lines sensitive to vemurafenib (MM074) or ^{V600E}BRAF melanoma cells lines with intrinsic resistance to vemurafenib (MM043).

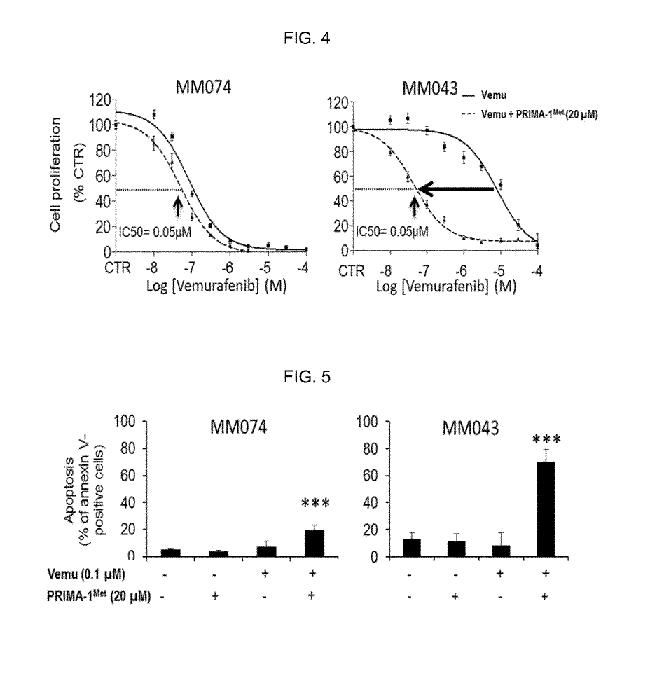

The effect of the combination of vemurafenib with PRIMA-1^{Met} on cell proliferation of sensitive (MM074) and intrinsically resistant (MM043) cells to vemurafenib are presented in FIG. 4. PRIMA-1^{Met} induced an impressive sensitization of the resistant cells to vemurafenib with a decrease of the IC50 of these resistant cells by about 20 folds (MM043, FIG. 4, right panel). By contrast, no additional significant effect of PRIMA-1^{Met} was observed in vemurafenib sensitive cells (MM074, FIG. 4, left panel).

The present inventors also examined apoptosis (Annexin V: PE Apoptosis Detection Kit I, BD Bioscience) in vemurafenib sensitive (MM074) and intrinsically resistant (MM043) cells to vemurafenib (FIG. 5). Vemurafenib and PRIMA-1^{Met} combination induced an important increase of apoptosis in cells with intrinsic resistance to vemurafenib compared with treatment with vemurafenib or PRIMA-1^{Met} alone (MM043, FIG. 5, right panel). Only, a marginal effect was observed in vemurafenib sensitive cells (MM074, FIG. 5, left panel).

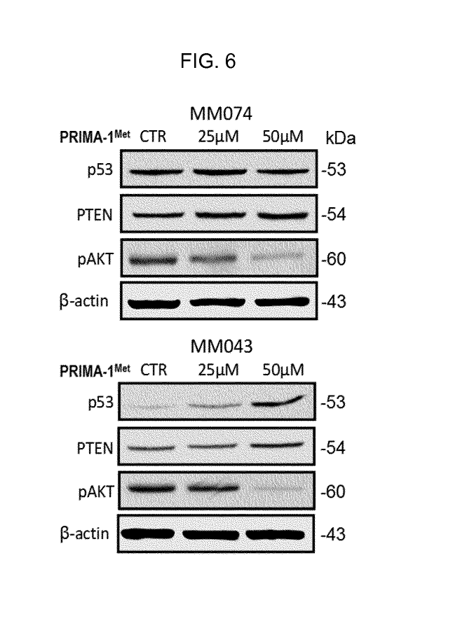

By comparing pathway effectors between sensitive (MM074) and intrinsically resistant (MM043) cells to vemurafenib, the present inventors found that treatment with PRIMA-1^{Met} restored p53 expression, stimulated PTEN expression and, consequently, inhibited AKT phosphorylation in cells with intrinsic resistance to vemurafenib (FIG. 6, bottom panels: MM043). By contrast, no significant effect was observed on p53 expression in vemurafenib sensitive cells (FIG. 6, top panels: MM074).

### Example 3

### Effect of Combination of Vemurafenib and the p53 Activator PRIMA-1^{Met} on ^{V600E}BRAF Melanoma Cells Lines with Acquired Resistance to Vemurafenib

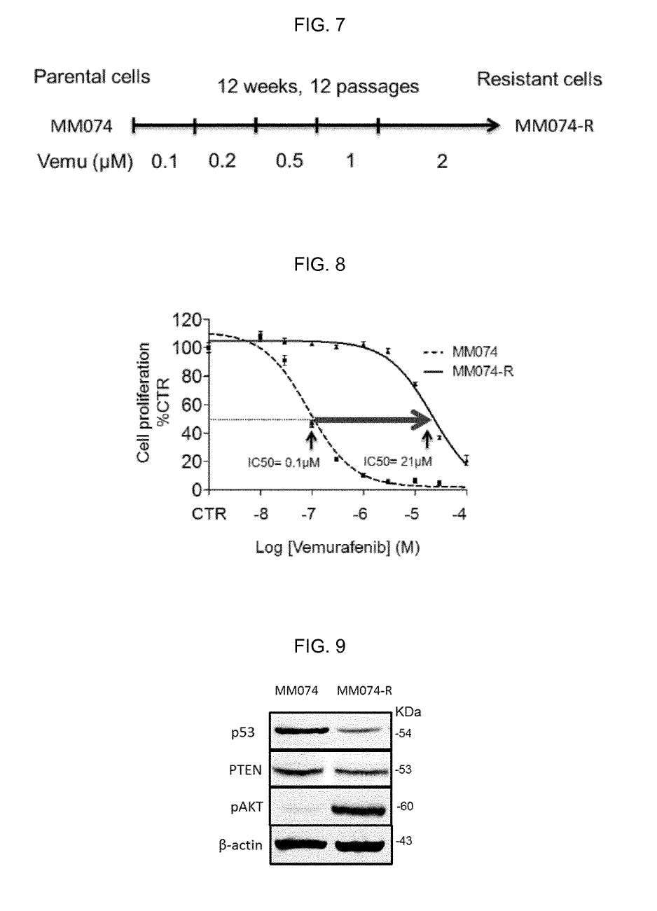

The MM074 cell line was made resistant (MM074-R) by a chronic exposure (12 weeks) to increasing concentrations (0.1 μM, 0.2 μM, 0.5 μM, 1 μM, and 2 μM) of vemurafenib (FIG. 7).

The results of cell proliferation of both parental and resistant cell lines are presented in FIG. 8. A 20-fold increase of IC50 occurs in cells with acquired resistance to vemurafenib compared to the sensitive/parental ones (FIG. 8, MM074: dashed line, MM074-R: full line).

By comparing pathway effectors between vemurafenib sensitive (MM074) and cells with acquired resistance (MM074-R), the latter was found associated with high AKT phosphorylation, low PTEN, and low p53 expression (FIG. 9, left panels: MM074; right panels: MM074-R). These important data indicate that the resistance could be acquired through the downregulation of p53.

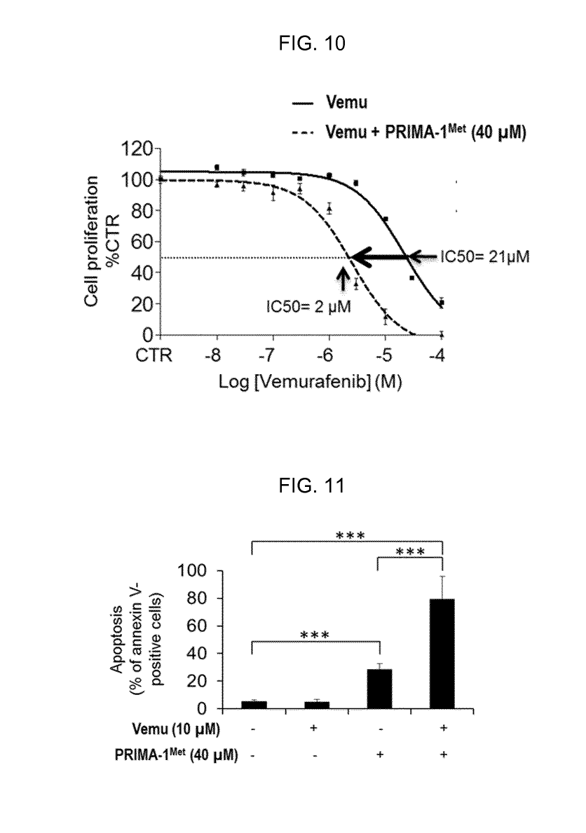

In order to break the acquired resistance by upregulating p53, PRIMA-1^{Met} was used in combination with vemurafenib. The results of such combination on cell proliferation of cells with acquired resistance to vemurafenib (MM074-R) are presented in FIG. 10 showing a 10 fold decrease of IC50.

Moreover, the combination of vemurafenib with PRIMA-1^{Met} induces a dramatic increase of apoptosis in cells with acquired resistance to vemurafenib compared with treatment with vemurafenib or PRIMA-1^{Met} alone (FIG. 11). Apoptosis was determined using Annexin V:PE Apoptosis Detection Kit I (BD Biosciences).

Importantly, it was observed that both intrinsic resistance (MM043, FIG. 4) and acquired resistance (MM074-R, FIG. 10) are very significantly reversed by p53 activation/upregulation using PRIMA-1^{Met}. The activation of p53 was systematically accompanied by an increase of PTEN expression and a consequent inhibition of AKT phosphorylation. Therefore, the data strongly suggest that sensitivity to vemurafenib requires an active crosstalk between ^{V600E}BRAF/MEK/ERK and PTEN/PI3K/AKT pathways leading to PTEN increase, while in resistant cells, such an effect can only be obtained by a concomitant ^{V600E}BRAF inhibition and PTEN stimulation best achieved by direct or indirect restoration of p53 intracellular levels.

### Example 4

### Direct Reactivation of p53 by PRIMA-1^{Met} Sensitizes Resistant Cells to Vemurafenib

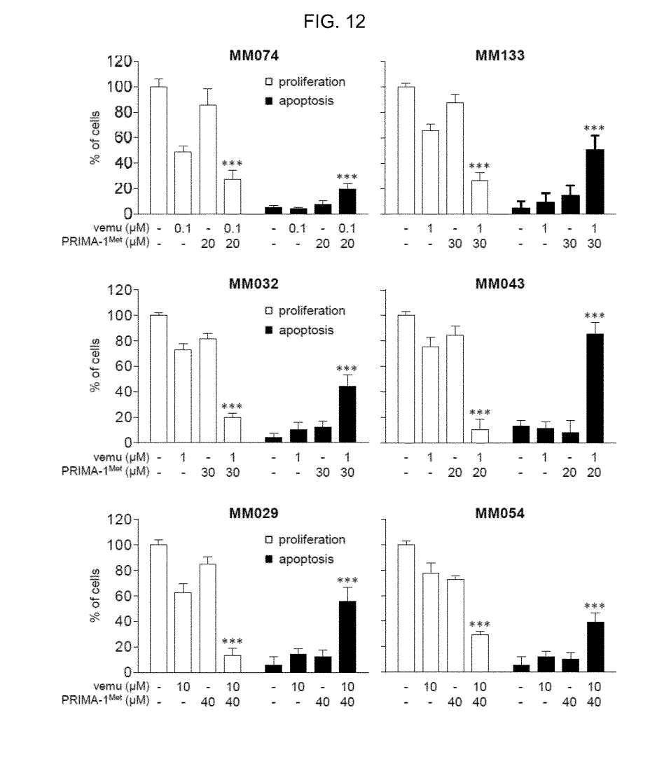

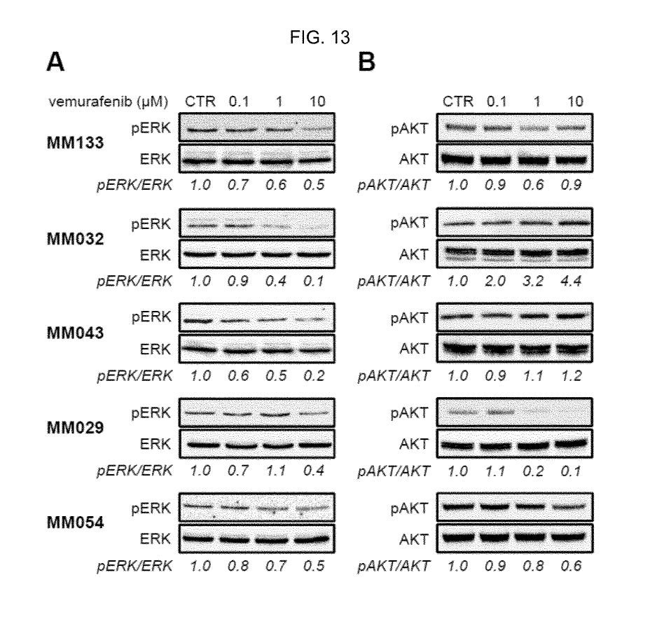

The five vemurafenib resistant melanoma lines were screened to their sensitivity to a combination of vemurafenib and PRIMA-1^{Met}. The latter was used at fixed concentrations (IC10) based on proliferation assays performed with PRIMA-1^{Met} alone and ranging from 20 to 40 μM depending on cell lines (data not shown). Importantly, we observed a strong synergistic effect on both cell proliferation inhibition and apoptosis induction (cf. FIG. 12 for comparison of the vemu-sensitive cell-line MM074,with the 5 vemu-resistant cell-lines and cf. “CI” in Table 1) with significant IC50s decrease in each case. As mechanisms that reactivate the MAPK and AKT pathways are commonly involved in resistance to oncogenic BRAF inhibitors, we evaluated the effect of vemurafenib for 24 hours on ERK and AKT phosphorylation and found that pERK was similarly inhibited in a concentration-dependent manner by the drug in all resistant lines, while pAKT was unaffected in three out of five resistant lines (MM032, MM043 and MM133) (FIG. 13).

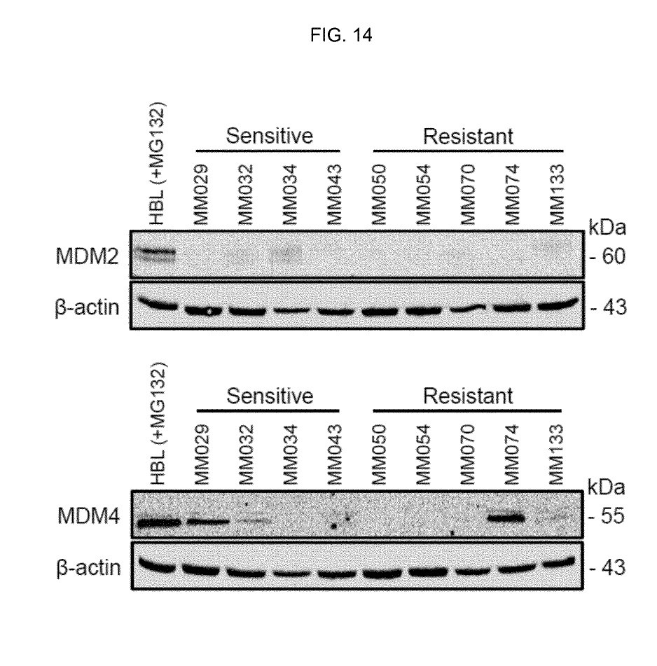

The direct p53 reactivation by Prima-1^{Met} was the best choice in all cell lines because the indirect reactivation through MDM2 or 4 would have been possible only in two. Indeed, In the five mutant BRAF vemurafenib-resistant melanoma lines, MDM2 was only weakly expressed in one (MM133), while MDM4 is detected in another (MM054) (FIG. 14)

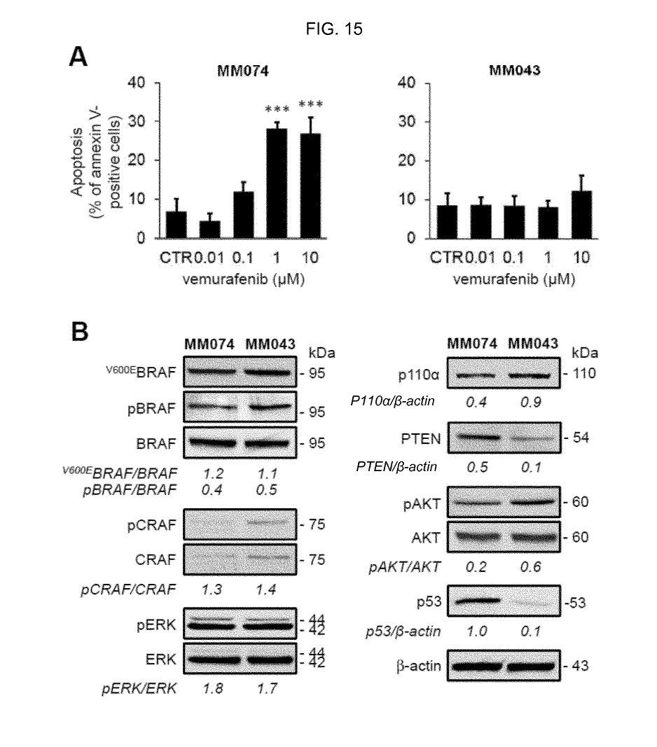

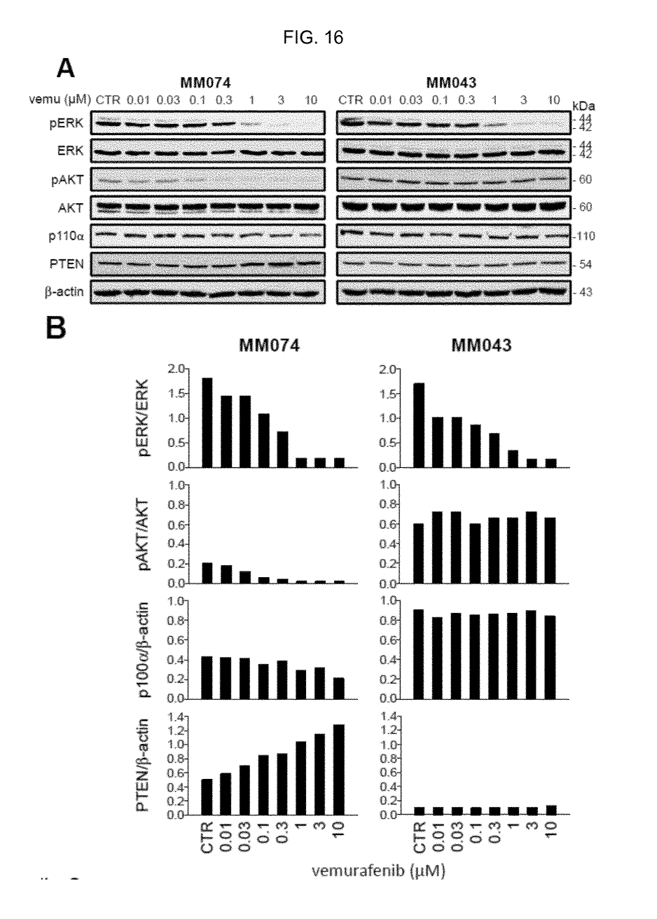

In order to investigate the mechanism underlying the synergistic effect of the vemurafenib/PRIMA-1^{Met} combination, we singled out two lines with varying sensitivity to this combination, one sensitive (MM074) and one resistant (MM043) (FIG. 15A). Of note, when comparing these two lines, we found that resistant cells had higher levels of p110-alpha (the catalytic subunit of PI3K) as well as pAKT and lower expression levels of both PTEN and p53 (FIG. 15B), possibly explaining its higher sensitivity to PRIMA-1^{Met} in the combination assay. We evaluated the concentration-dependent effect of vemurafenib on the same parameters and found that pERK was similarly inhibited by the drug in both sensitive and resistant lines (FIG. 16A). Moreover, in the sensitive cells, pAKT was strongly inhibited and accompanied by a concentration-dependent increase of PTEN expression while, in the resistant ones, neither AKT phosphorylation nor PTEN expression were affected even at drug concentration as high as 10 μM (FIG. 16B). These data suggest that the loss of an active crosstalk between BRAF/MEK/ERK and PI3K/PTEN/AKT pathways may affect the sensitivity to the BRAF inhibitor and confirm that a sustained activation of the PI3K/AKT pathway confers resistance to vemurafenib (Atefi M et al., 2011, PLoS ONE, 6(12):e28973; Gopal YNV et al., 2014, Cancer Res. 74(23):7037-7047; Su F et al., 2012, Cancer Res. 72(4):969-978.).

### Example 5

### Reactivation of p53 by PRIMA-1^{Met} is Associated with p110α/AKT Inhibition and PTEN Upregulation

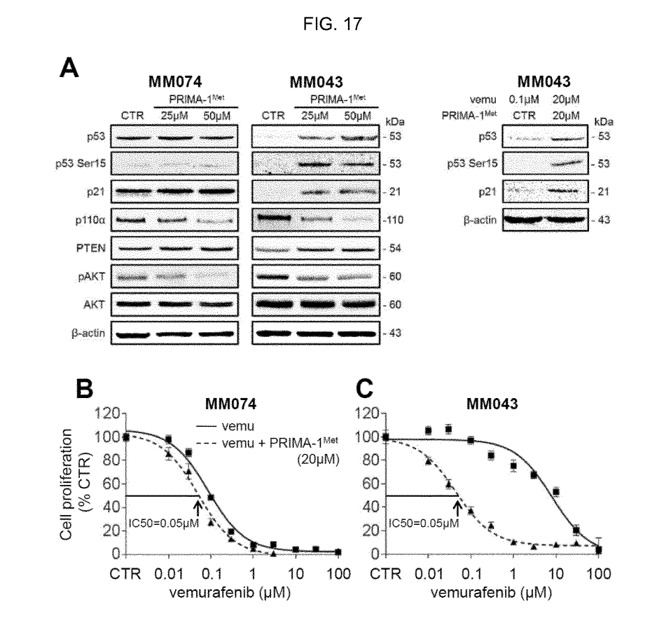

As PI3K and PTEN are both p53 targets (Astanehe A et al., 2008, J Cell Sci. 121(Pt 5):664-674; Stambolic V et al., 2001, Mol Cell. 8(2):317-325.), p53 reactivation may inhibit PI3K/AKT pathway and contribute to apoptosis promotion. We first checked this hypothesis by exposing both sensitive (MM074) and resistant (MM043) cells to 25 and 50 μM PRIMA-1^{Met}, and observed the activation of the p53 pathway (phosphorylation of p53 at Ser15 and stimulation of p21 expression), an increase in PTEN levels as well as an inhibition of p110α expression and AKT phosphorylation (FIG. 17A). Importantly, exposure to PRI MA-1^{Met} led to a 200-fold decrease in IC50 to vemurafenib in the resistant line (FIG. 17C, Table 1) and a more modest (2-fold) but significant decrease in the sensitive one (FIG. 17B, Table 1).

### Example 6

### Synergistic Inhibition of Melanoma Cell Growth by Combining Mutant BRAF and Selective PI3K/mTOR Inhibitors

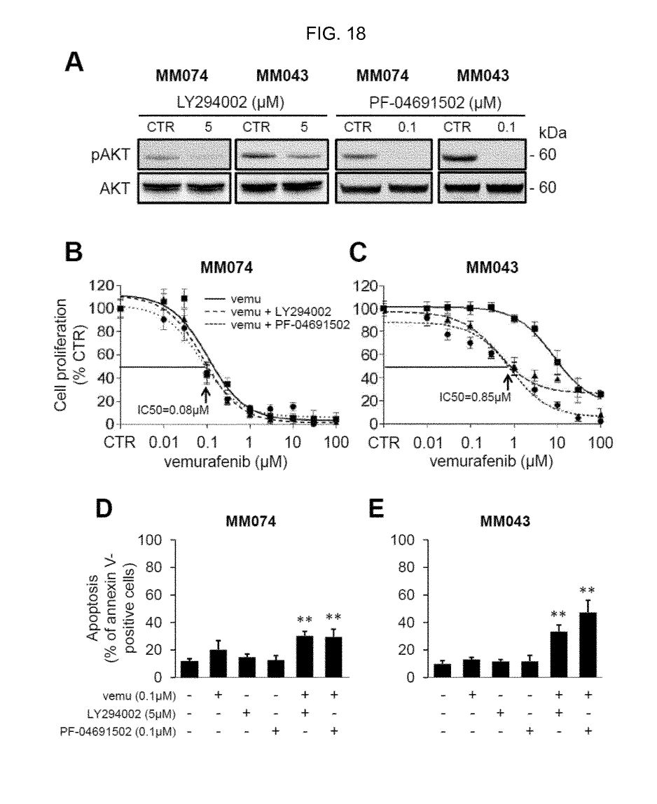

In order to compare the cytotoxic effect of p53 reactivation with PI3K/AKT/mTOR inhibition on vemurafenib-exposed cells, we used two different specific inhibitors of the PI3K/AKT pathway, the PI3K inhibitor LY294002 and the PI3K/mTOR dual inhibitor PF-04691502, and examined cell proliferation and apoptosis. We first established that the IC10s of both sensitive (MM074) and resistant (MM043) melanoma lines to LY294002 and PF-04691502 were, respectively, 5 and 0.1 μM. AKT phosphorylation was inhibited at those concentrations (FIG. 18 A) that were used in combination with increasing concentrations of vemurafenib. In sensitive cells, proliferation was not significantly affected (FIG. 18B), but apoptosis was increased (FIG. 18D), while, in resistant ones, a significant synergistic inhibition of cell proliferation (IC50 more than 12-fold lower, CI were 0.64 for PF-04691502 and 0.56 for LY294002) (FIG. 18C) and a significant increase in apoptosis (FIG. 18E) were observed. This further supports that the resistance to vemurafenib is effectively due to an activated PI3K/AKT pathway in the MM043 line. Notably, this synergistic effect remains by far lower than the one observed above when combining vemurafenib and PRI MA-1^{Met}.

### Example 7

### p53 Reactivation Overcomes Acquired Resistance to Vemurafenib

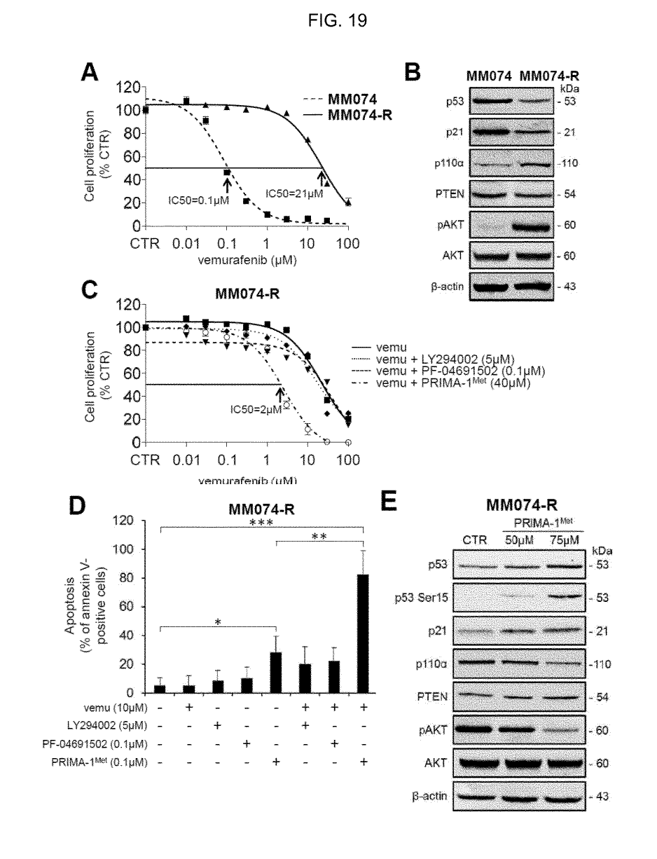

Acquired resistance has been obtained in the vemurafenib sensitive line MM074 by a 12-week of exposure to gradually increasing concentrations (0.1-2 μM) of the drug. Acquired resistance to vemurafenib translated into a 210-fold increase of IC50 in resistant cells (MM074-R) as compared to parental cells (MM074) (FIG. 19 A, Table 1). The resistance was associated with a significant downregulation of p53, p21 and PTEN levels along with a substantial increase in p110α expression and consequently AKT phosphorylation (FIG. 19 B).

We determined IC10s for both PI3K/mTOR inhibitors (5 μM LY294002, 0.1 μM PF-04691502) or p53 activator (40 μM PRIMA-1^{Met}) in MM074-R. Of the latter, only PRIMA-1^{Met} could very significantly affect cell proliferation (FIG. 19C), reporting a 10-fold lower IC50 (Table 1, CI was 0.05) associated with a massive induction of apoptosis (FIG. 19D) when combined to vemurafenib. This was preceded by a dose-dependent increase of p53 expression and phosphorylation, upregulation of p21 and PTEN levels and decrease of p110α expression and AKT phosphorylation (FIG. 19E).

### Example 8

### In Vivo Effect of Combination of Vemurafenib and the p53 Activator PRIMA-1^{Met} on ^{V600E}BRAF Melanoma Cells Line with Intrinsic Resistance to Vemurafenib

The effect of vemurafenib and PRIMA-1^{Met} alone and in combination was evaluated on xenograft growth of the resistant MM043 cells (4 groups: control, vemurafenib, PRIMA-1^{Met}, and vemurafenib+PRIMA-1^{Met}). 10⁷ cells were implanted (by injection) in Swiss nudes (10 mice per experimental condition). Tumor growth was monitored over 3 weeks to reach 5-8 mm diameter. Then, effectors were intraperitoneally (IP) administered daily by IP injection (45 mg/kg vemurafenib and/or 50 mg/kg PRIMA-1^{Met}). The tumor volume was measured every 2 days for 3 weeks. At the end of experiments, all mice were sacrificed. Tumors and residual lesions will be embedded into paraffin for further IHC analyses (p53, PTEN and pAKT).

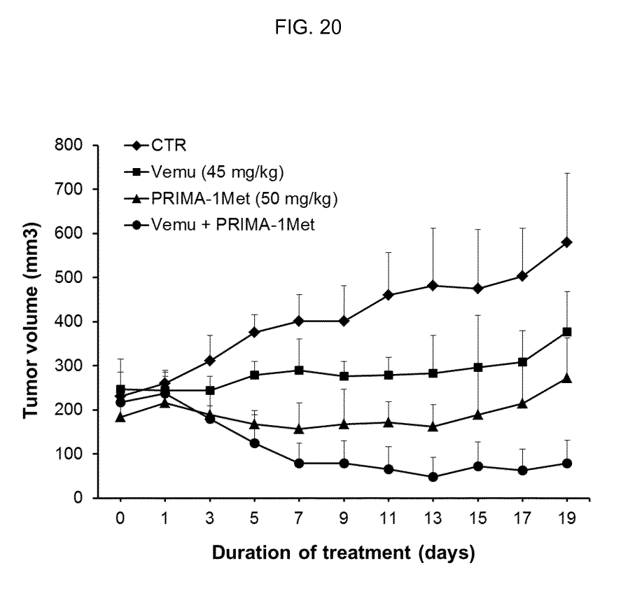

This first animal study validated the in vitro observations. The data indicate that only the combination of vemurafenib and PRIMA-1^{Met} caused tumor regression and was more effective to inhibit tumor growth when compared with vemurafenib or PRIMA-1^{Met} alone (FIG. 20).

### Example 9

### PRIMA-1^{Met} and Vemurafenib Synergize to Inhibit the Growth of Vemurafenib-Resistant Melanoma Xenografts

The effect of vemurafenib and PRIMA-1^{Met} alone and in combination was evaluated on the in vivo growth of the vemurafenib-resistant MM043 and MM074-R melanoma cells. The sensitive melanoma cells (MM074) xenografts were used as control. After subcutaneous cell injection, tumor growth was monitored to reach volumes of about 200 mm³. Then, effectors were daily intraperitoneally administered (45 mg/kg vemurafenib and/or 50 mg/kg PRIMA-1^{Met}). We used doses of vemurafenib and PRIMA-1^{Met} that, alone, did not cause any major inhibition of tumor growth.

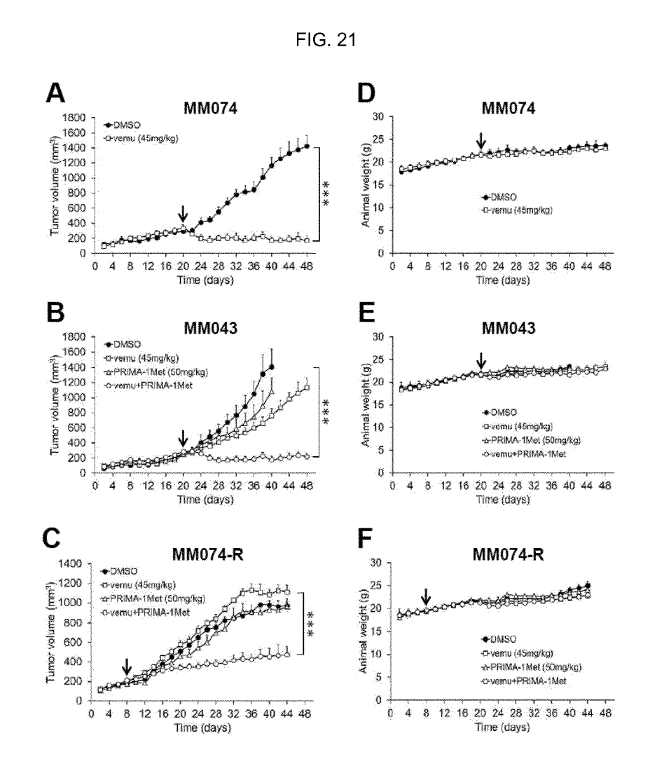

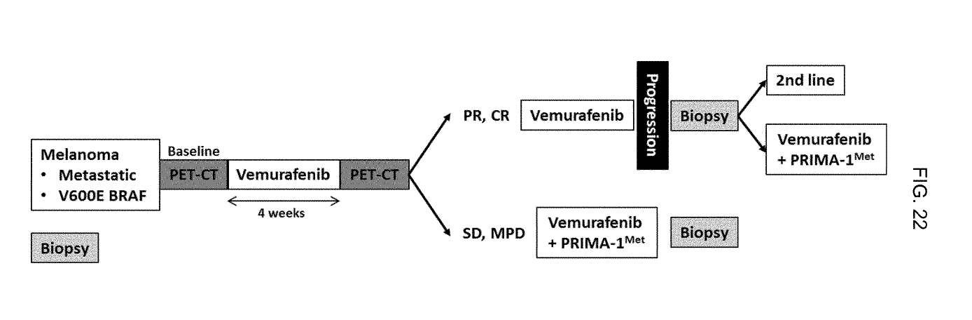

In vemurafenib-sensitive (MM074) xenografts, vemurafenib alone inhibited tumor growth as of day 4 after starting treatment. The average tumor volume in control animals treated with vehicule (DMSO) for 28 days was ˜1425 mm3 (n=10), while it was ˜190 mm3 for animals that received vemurafenib (FIG. 21A). In intrinsically vemurafenib-resistant (MM043) xenografts, the mean tumor volumes in animals treated with vehicle, vemurafenib and PRIMA-1^{Met} were, respectively, ˜1410 mm³ after 20 days, ˜1130 mm³ after 28 days and 1080 mm³ after 20 days of treatment.

Compared to each effector alone, vemurafenib and PRIMA-1^{Met} combination produced a complete suppression of tumor growth starting at day 8 after treatment initiation comparable to that observed in sensitive cells. With the combination, the average tumor volume significantly dropped to ˜220 mm³ after 28 days of treatment (FIG. 21B). Of note, vemurafenib alone did not significantly affected tumor growth. In MM074-R xenografts with acquired resistance to vemurafenib, only vemurafenib and PRIMA-1^{Met} combination could efficiently suppress tumor growth over the whole period of treatment of 36 days (FIG. 21C).

No significant difference in body weight was observed in all three conditions indicating that these treatment regimens did not lead to overall toxicity (FIG. 21D-F).

### Example 10

### Phase I Clinical Study of the Combination Regimen of Vemurafenib and the p53 Activator PRIMA-1^{Met} on ^{V600E}BRAF Melanoma Cells Lines

Based on the aforementioned in vitro data and animal in vivo data, a phase I/II clinical trial is initiated combining vemurafenib and PRIMA-1^{Met} in melanoma patients who do not respond to vemurafenib. Stage IV melanoma patients >17 years, with metastases harboring the mutant ^{V600E/K}BRAF (as detected by Cobas, Roche and validated by IHC using VE-1 antibody), progressive or having presented a non-heterogeneous response after four weeks of vemurafenib as assessed by 2-fluorodeoxy-D-glucose Positron emission tomography (FDG-PET) using guidelines developed at our institution (Hendlisz et al., 2012, Ann. Oncol., 23, 1687-1693), are proposed to be included in the clinical study (FIG. 22).

Before vemurafenib treatment initiation and when possible, one cutaneous or lymph node metastasis is removed for further lab studies. The tumor tissue is split into 3 parts: the first part is paraffin-embedded for pathological evaluation (e.g., mutational status), the second part is used to establish primary cultures (e.g., to determine intrinsic and acquired resistance and its underlying mechanism), and the third part is snap frozen for translational research.

A second metastatic tissue is collected at the time of cancer progression. Half of the second metastatic tissue is paraffin-embedded for immunohistochemistry (IHC) and sequencing, and the other half is snap frozen for translational research (e.g., kinome profiling).

The clinical study can provide further insight in the effect of the combination of vemurafenib and PRIMA-1^{Met} in melanoma patients with resistance to vemurafenib.

### Example 11

### In Vitro Diagnostic Model to Predict Sensitivity or Resistance of Patients to Vemurafenib Alone or in Combination with PRIMA-1^{Met}

The present inventors have developed an in vitro diagnostic model to predict responsiveness (sensitivity or resistance) of patients to vemurafenib alone or in combination with PRIMA-1^{Met}.

For each patient included in the trial, a cell line is established (when possible) according to standard protocols and QC (Morandini et al., 1998, J. Cell. Physiol., 175, 276-282). To speed-up the process, tumor cell enrichment techniques (fibroblast depletion, MACS Miltenyl Biotec) are performed and melanoma cells are characterized by immunocytochemistry (ICC) for Melan-A (melanocyte lineage marker) and ^{V600E}BRAF expression. This step is preferably performed within one month of sampling.

Cytotoxicity of vemurafenib and PRIMA-1^{Met} is assessed by crystal violet staining and apoptosis assay to calculate IC50 and IC10. The latter is used for drug combination studies. The result reflects a prediction of the response in the corresponding patient (from whom the culture is derived). The next step is a validation step correlating the extent of sensitivity or resistance of cells and objective clinical responses in patients.

If the cells are found intrinsically resistant to vemurafenib, p53, PTEN and pAKT expression is examined by ICC to explore the possibility to combine vemurafenib with PRIMA-1^{Met} and use it in the patient.

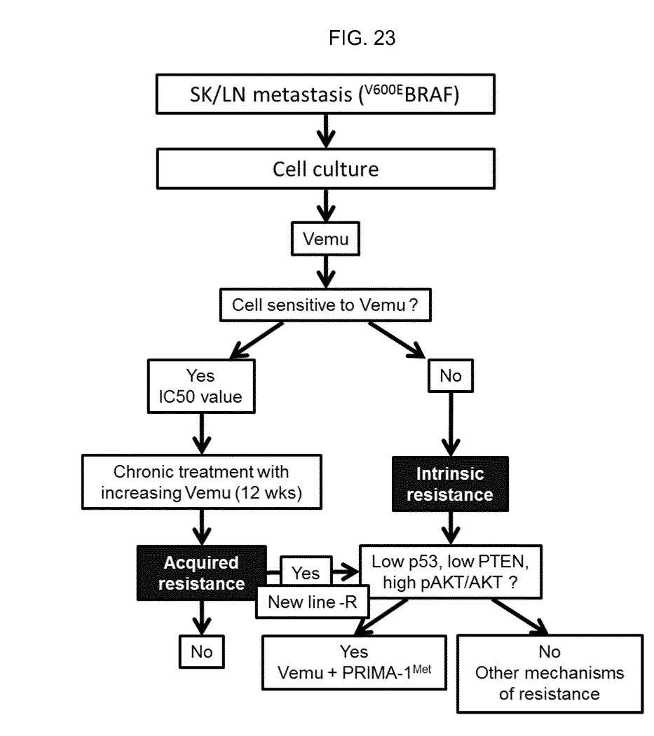

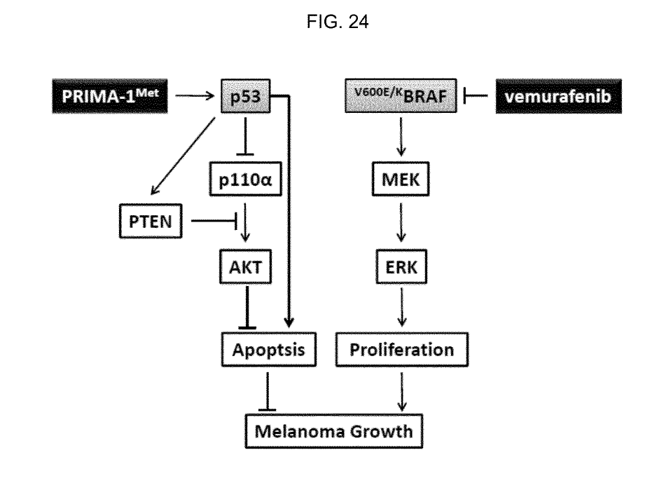

If cells are sensitive to vemurafenib, IC50 value is calculated and cells are subjected to a chronic treatment (12 weeks) with increasing drug concentrations (0.1-2 μM) in order to develop acquired resistance. When done, a specific profile often associated with resistance is searched for: low p53, low PTEN, and high pAKT. Next, various vemurafenib and PRIMA-1^{Met} combinations are tested in order to propose the appropriate combination and dosing to the corresponding patient (from whom the culture is derived). The scheme is shown in FIG. 23.

1. A method of treating melanoma in a patient, comprising the step of administering to said patient, a therapeutically effective amount of a p53-activating agent capable of transferring wild-type tumor protein p53 (p53) from an inactive conformation into an active conformation capable of inducing apoptosis, simultaneously or sequentially with the administration of a BRAF-inhibiting agent capable of inhibiting activity of serine/threonine-protein kinase B-Raf (BRAF) comprising an activating mutation.

2. The method according to claim 1, wherein said p53-activating agent is administered before or after administration of said BRAF-inhibiting agent.

3. The method according to claim 1 or 2, wherein said p53-activating agent is administered simultaneously with the administration of the BRAF-inhibiting agent.

4. The method according to any one of claims 1 to 3, wherein said p53-activating agent is a compound having the structure of Formula I, or a pharmaceutically acceptable salt or prodrug thereof, 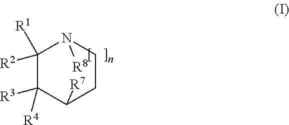
wherein
n is 0,1 or 2;
R¹ and R² are the same or different and are selected from —H, —CH₂—R⁵, —CH₂—O—R⁵, —CH₂—S—R⁵, —CH₂—NH—R⁵, —COO—R⁵, —CO—NH—R⁵, —CH₂—NH—CO—R⁵, —CH₂—O—CO—R⁵, —CH₂—NH—CO—NHR⁵, —CH₂—NH—CO—OR⁵, —CH₂—NH—CS—NHR⁵ and —CH₂—O—CO—NHR⁵; or R¹ and R² are together ═CH₂;
R³ and R⁴ are the same or different and are selected from —H, —OH, —SH, —NH2, —NHR⁵ and —O—CO—C₆H₅; or R³ and R⁴ together are ═O, ═S, =NH or ═NR⁵;
R⁵ represents the same or different groups selected from H, substituted or non-substituted C₁₋₁₀alkyl, C₂₋₁₀alkenyl, C₂₋₁₀alkynyl, substituted or non-substituted C₃₋₁₂dycloalkyl, substituted or non-substituted benzyl groups, substituted or non-substituted aryl or mono-, bi-, tricyclic unsubstituted or substituted heteroaromatic ring(s) with one or more heteroatoms and non-aromatic heterocycles wherein the substituents of the substituted groups are selected from C₁₋₁₀alkyl, C₂₋₁₀alkenyl, C₂₋₁₀alkynyl, halogen, substituted or non-substituted aryl, substituted or non-substituted heteroaromatic compounds, non-aromatic heterocycles, C₁₋₁₀alkyloxy, C₁₋₁₀alkylamino, C₂₋₁oalkenylamino, C₂₋₁₀alkynylamino, COR⁶, CONR⁶ and COOR⁶;
R⁶ is selected from H, unsubstituted or substituted C₁₋₁₀alkyl, C₂₋₁₀alkenyl or alkynyl, benzyl, aryl, unsubstituted or substituted heteroaromatic rings with one or more heteroatoms and non-aromatic heterocycles;
R⁷ and R⁸ together form a bridging CH₂—CH₂ moiety; or R⁷ and R⁸ are both hydrogen; or wherein said p53-activating agent is CDB3, SCH529074, NSC319726, or CP-31398.

5. The method according to any one of claims 1 to 4, wherein said p53-activating agent is a compound selected from the group consisting of 2-hydroxymethyl-2-methoxymethylazabicyclo[2.2.2]octan-3-one, 2 ,2-bis(hyd roxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, and 2-(2,6-dichloro-9-methylenepurine)-3-quinuclidinone, or a pharmaceutically acceptable salt thereof.

6. The method according to any one of claims 1 to 5, wherein said BRAF-inhibiting agent is a compound having the structure of Formula III, or a pharmaceutically acceptable salt or prodrug thereof, 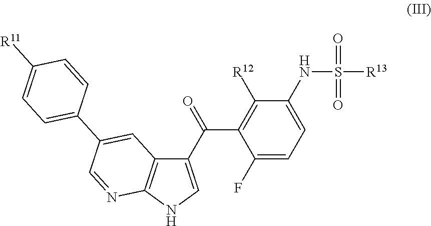
wherein
R¹¹ is selected from the group consisting of hydrogen, halogen, optionally substituted C₁₋₆alkyl, optionally substituted C₁₋₆alkenyl, optionally substituted C₁₋₆alkynyl, optionally substituted cycloalkyl, optionally substituted heterocycloalkyl, optionally substituted aryl, optionally—substituted heteroaryl, —OH, —NH₂, —CN, —NO₂, —O(O)OH, —S(O)₂NH₂, —C(O)NH₂, —C(S)NH₂, —NHC(O)NH₂, —NHC(S)NH₂, —NHS(O)₂NH₂, —OR¹⁴, —SR¹⁴, —NR¹⁵R¹⁴, —C(O)R¹⁴, —C(S)R¹⁴, —O(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —N R¹⁵S(O)₂R¹⁴, —NR¹⁵O(O)N H₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, and —S(O)₂R¹⁵;
R¹² is selected from the group consisting of hydrogen, fluoro and chloro;
R¹³ is selected from the group consisting of optionally substituted C₂₋₆alkyl, optionally substituted aryl, optionally substituted heteroaryl, and NR¹⁶R¹⁷;
R¹⁴ is selected from the group consisting of optionally substituted C₁₋₆-alkyl, optionally substituted C₁₋₆-alkenyl, provided, however, that when R¹⁴ is optionally substituted C₁₋₆-alkenyl, no alkene carbon thereof is bound to N, S, O, S(O), S(O)₂, C(O) or C(S) of —OR¹⁴, —SR¹⁴, —NR¹⁵R¹⁴, —O(O)R¹⁴, —O(S)R¹⁴, —O(O)OR¹⁴, —O(O)NR¹⁵R¹⁴, —O(S)NR¹⁵R¹⁴, —S(O)₂NR¹⁵R¹⁴, —NR¹⁵O(O)R¹⁴, —NR¹⁵O(S)R¹⁴, —NR¹⁵S(O)₂R¹⁴, —NR¹⁵O(O)NH₂, —NR¹⁵O(O)NR¹⁵R¹⁴, —NR¹⁵C(S)NH₂, —NR¹⁵O(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted C₁₋₆alkynyl, provided, however, that when R¹⁴ is optionally substituted C₁₋₆alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —O(O)OR¹⁴, —C(O)NR¹⁵R¹⁴, —C(S)NR¹⁵R¹⁴, —S(O)₂NR¹⁵R¹⁴, —NR¹⁵O(O)R¹⁴, —NR¹⁵O(S)R¹⁴, —NR¹⁵S(O)₂R¹⁴, —NR¹⁵C(O)NH₂, —NR¹⁵O(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted cycloalkyl, optionally substituted heterocycloalkyl, optionally substituted aryl, and optionally substituted heteroaryl;
R¹⁵ is selected from the group consisting of hydrogen and optionally substituted C₁₋₆alkyl; and
R¹⁶ and R¹⁷ are independently hydrogen or optionally substituted C₁₋₆alkyl, or R¹⁶ and
R¹⁷ combine with the nitrogen to which they are attached to form optionally substituted 5-6 membered heterocycloalkyl.

7. The method according to any one of claims 1 to 5, wherein said BRAF-inhibiting agent is a compound selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-[5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1H-imidazol-2-yl]-4-pyridyl]oxy]-N-[4-(trifluoromethyl)phenyl]benzimidazol-2-amine.

8. The method according to any one of claims 1 to 7, wherein said p53-activating agent is 2-hydroxymethyl-2-methoxymethylazabicyclo[2.2.2]octan-3-one (PRIMA-1^{Met}) and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib); or
wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is SCH529074 (Demma M, et al., 2010, J Biol Chem. 285(14):10198-10212) and said BRAF-inhibiting agent is N-(3-{[5-(4-chlorophenyl)-1H-pyrrolo[2,3-b]pyrid in-3-yl]carbonyl}-2,4-difluorophenyl)-propane-1-sulfonamide (vemurafenib); or
wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is NSC319726 (Yu X. et al., 2012, Cancer Cell. 15;21(5):614-25) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib).

9. The method according to any one of claims 1 to 8, wherein said p53-activating agent and said BRAF-inhibiting agent are comprised in a composition or in a kit of parts, preferably in a pharmaceutical composition or in a pharmaceutical kit of parts.

10. The method according to any one of claims 1 to 9, wherein the melanoma comprises expression of BRAF comprising an activating mutation, preferably wherein the melanoma comprises (a) cell(s) comprising expression of ^{V600E/K}BRAF.

11. The method according to any one of claims 1 to 10, wherein the melanoma comprises (a) cell(s) with intrinsic or acquired resistance to said BRAF-inhibiting agent.

12. A method of treating melanoma resistant to N-(3-{[5-(4-chlorophenyl)-1H-pyrrolo[2,3-b]pyridin-3-yl]carbonyl}-2,4-difluorophenyl) propane-1-sulfonamide (vemurafenib) in a patient, comprising the administration of a therapeutically effective amount of a p53-activating agent capable of transferring wild-type tumor protein p53 from an inactive conformation into an active conformation capable of inducing apoptosis.

13. The method according to claim 12, wherein said resistance is pre-existing, or is acquired due to (chronic) treatment with N-(3-{[5-(4-chlorophenyl)-1H-pyrrolo[2,3-b]pyridin-3-yl]carbonyl}-2,4-difluorophenyl) propane-1-sulfonamide (vemurafenib).

14. A method of treating melanoma in a patient, comprising the administration of a pharmaceutical composition comprising: a p53-activating agent capable of transferring wild-type p53 from an inactive conformation thereof into an active conformation capable of inducing apoptosis and a BRAF-inhibiting agent capable of inhibiting activity of BRAF comprising an activating mutation, for use in treating melanoma.

15. The method according to claim 14, wherein said p53-activating agent is 2-hydroxymethyl-2-methoxymethylazabicyclo[2.2.2]octan-3-one (PRIMA-1^{Met}) and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib); or
wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is SCH529074 (Demma M, et al., 2010, J Biol Chem. 285(14):10198-10212) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is NSC319726 (Yu X. et al., 2012, Cancer Cell. 15;21(5):614-25) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib).

16. The method according to any one of claims 1 to 15, wherein said p53-activating agent and said BRAF-inhibiting agent are comprised in a composition or in a kit of parts, preferably in a pharmaceutical composition or in a pharmaceutical kit of parts.

17. The method according to any one of claims 1 to 15, wherein said p53-activating agent may be administered simultaneously or sequentially with said BRAF-inhibiting agent and with a MEK-inhibiting agent capable of inhibiting activity of mitogen-activated protein kinase kinase 1 (MEK 1) and/or mitogen-activated protein kinase kinase 2 (MEK2).

18. The method according to claim 17, wherein said p53-activating agent, said BRAF-inhibiting agent and said MEK-inhibiting agent are comprised in a composition or in a kit of parts, preferably in a pharmaceutical composition or in a pharmaceutical kit of parts.

19. A p53-activating agent capable of transferring wild-type tumor protein p53 (p53) from an inactive conformation into an active conformation capable of inducing apoptosis, for use in the treatment of melanoma, wherein said p53-activating agent is administered simultaneously or sequentially with a BRAF-inhibiting agent capable of inhibiting activity of serine/threonine-protein kinase B-Raf (BRAF) comprising an activating mutation.

20. The p53-activating agent for use according to claim 19, wherein said p53-activating agent is administered before or after administration of said BRAF-inhibiting agent.

21. The p53-activating agent for use according to claim 19 or 20, wherein said p53-activating agent is administered simultaneously with the administration of the BRAF-inhibiting agent.

22. The p53-activating agent for use according to any one of claims 19 to 21, wherein said p53-activating agent is a compound having the structure of Formula I, or a pharmaceutically acceptable salt or prodrug thereof, 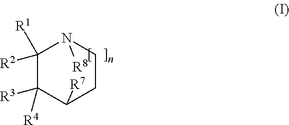
wherein
n is 0,1 or 2;
R¹ and R² are the same or different and are selected from —H, —CH₂—R⁵, —CH₂—O—R⁵, —CH₂—S—R⁵, —CH₂—NH—R⁵, —COO—R⁵, —CO—NH—R⁵, —CH₂—NH—CO—R⁵, —CH₂—O—CO—R⁵, —CH₂—NH—CO—NHR⁵, —CH₂—NH—CO—OR⁵, —CH₂—NH—CS—NHR⁵ and —CH₂—O—CO—NHR⁵; or R¹ and R² are together ═CH₂;
R³ and R⁴ are the same or different and are selected from —H, —OH, —SH, —NH2, —NHR⁵ and —O—CO—C₆H₅; or R³ and R⁴ together are ═O, ═S, ═NH or ═NR⁵;
R⁵ represents the same or different groups selected from H, substituted or non-substituted C₁₋₁₀alkyl, C₂₋₁₀alkenyl, C₂₋₁₀alkynyl, substituted or non-substituted C₃₋₁₂dycloalkyl, substituted or non-substituted benzyl groups, substituted or non-substituted aryl or mono-, bi-, tricyclic unsubstituted or substituted heteroaromatic ring(s) with one or more heteroatoms and non-aromatic heterocycles wherein the substituents of the substituted groups are selected from C₁₋₁₀alkyl, C₂₋₁₀alkenyl, C₂₋₁₀alkynyl, halogen, substituted or non-substituted aryl, substituted or non-substituted heteroaromatic compounds, non-aromatic heterocycles, C₁₋₁₀alkyloxy, C₁₋₁₀alkylamino, C₂₋₁₀alkenylamino, C₂₋₁₀alkynylamino, COR⁶, CONR⁶ and COOR⁶;
R⁶ is selected from H, unsubstituted or substituted C₁₋₁₀alkyl, C₂₋₁₀alkenyl or alkynyl, benzyl, aryl, unsubstituted or substituted heteroaromatic rings with one or more heteroatoms and non-aromatic heterocycles;
R⁷ and R⁸ together form a bridging CH₂—CH₂ moiety; or R⁷ and R⁸ are both hydrogen; or wherein said p53-activating agent is CDB3, SCH529074, NSC319726, or CP-31398.

23. The p53-activating agent for use according to any one of claims 19 to 22, wherein said p53-activating agent is a compound selected from the group consisting of 2-hydroxymethyl-2-methoxymethylazabicyclo[2.2.2]octan-3-one, 2,2-bis(hydroxymethyl)-1-azabicyclo[2.2.2]octan-3-one, 9-(azabicyclo[2.2.2]octan-3-one)-6-chloro-9H-purine, 2-(hydroxymethyl)quinuclidine-3,3-diol, 2-(adenine-9-methylene)-3-quinuclidinone, 2-methylene-3-quinuclidinone, 2-(2-amino-3-chloro-5-trifluoromethyl-1-methylaniline)-3-quinuclidinone, 2-(6-trifluoromethyl-4-chlorobenzimidazole-l-methylene)-3-quinuclidinone, 2-(6-methoxypurine-9-methylene)-3-quinuclidinone, 2-(8-azaadenine-9-methylene)-3-quinuclidinone, 1-azabicyclo[2.2.2]oct-3-ylbenzoate, 2-(5,6-dimethyl-benzimidazole-1-methylene)-3-quinuclidinone, 2-(8-azaadenine-7-methylene)-3-quinuclidinone, 2-(7-methylene-1,3-dimethyluric acid)-3-quinuelidinone, and 2-(2,6-dichloro-9-methylenepurine)-3-quinuclidinone, or a pharmaceutically acceptable salt thereof.

24. The p53-activating agent for use according to any one of claims 19 to 23, wherein said BRAF-inhibiting agent is a compound having the structure of Formula III, or a pharmaceutically acceptable salt or prodrug thereof, 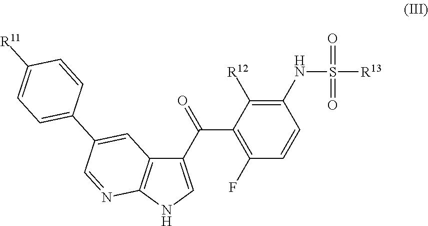
wherein
R¹¹ is selected from the group consisting of hydrogen, halogen, optionally substituted C₁₋₆alkyl, optionally substituted C₁₋₆alkenyl, optionally substituted C₁₋₆alkynyl, optionally substituted cycloalkyl, optionally substituted heterocycloalkyl, optionally substituted aryl, optionally substituted heteroaryl, —OH, —NH₂, —CN, —NO₂, —C(O)OH, —S(O)₂NH₂, —C(O)NH₂, —C(S)NH₂, —NHC(O)NH₂, —NHC(S)NH₂, —NHS(O)₂NH₂, —OR¹⁴, —SR¹⁴, —NR¹⁵R¹⁴, —C(O)R¹⁴, —C(S)R¹⁴, —C(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)N H₂, —NR¹⁵C(O)NR¹⁵R¹⁴, —NR¹⁵C(S)N H₂, —NR¹⁵C(S)NR¹⁵R¹⁴, —NR¹⁵S(O)₂NH₂, —NR¹⁵S(O)₂NR¹⁵R¹⁴, —S(O)R¹⁵, and —S(O)₂R¹⁵.
R¹² is selected from the group consisting of hydrogen, fluoro and chloro;
R¹³ is selected from the group consisting of optionally substituted C₂₋₆alkyl, optionally substituted aryl, optionally substituted heteroaryl, and NR¹⁶R¹⁷;
R¹⁴ is selected from the group consisting of optionally substituted C₁₋₆-alkyl, optionally substituted C₁₋₆-alkenyl, provided, however, that when R¹⁴ is optionally substituted C₁₋₆-alkenyl, no alkene carbon thereof is bound to N, S, O, S(O), S(O)₂, C(O) or C(S) of —OR¹⁴, —SR¹⁴, —NR¹⁵R¹⁴, —C(O)R¹⁴, —C(S)R¹⁴, —O(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR¹⁵C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted C₁₋₆alkynyl, provided, however, that when R¹⁴ is optionally substituted C₁₋₆alkenyl, no alkene carbon thereof is bound to N, S, O, S(O), S(O)₂, C(O) or C(S) of —OR¹⁴, —SR¹⁴, —NR¹⁵R¹⁴, —C(O)_{R}¹⁴, —C(S)R¹⁴, —O(O)OR¹⁴, —C(O)NR¹⁵R¹⁴, —C(S)NR¹⁵R¹⁴, —S(O)₂NR¹⁵R¹⁴, —NR¹⁵C(O)R¹⁴, —NR¹⁵C(S)R¹⁴, —NR¹⁵S(O)₂R¹⁴, —NR15C(O)NH₂, —NR¹⁵C(O)NR¹⁵R¹⁴, —NR¹⁵C(S)NH₂, —NR¹⁵C(S)NR¹⁵R¹⁴, —NR¹⁵S(O)₂NH₂, —NR¹⁵S(O)₂NR¹⁵R¹⁴, —S(O)R¹⁵, or —S(O)₂R¹⁵, optionally substituted cycloalkyl, optionally substituted heterocycloalkyl, optionally substituted aryl, and optionally substituted heteroaryl;
R¹⁵ is selected from the group consisting of hydrogen and optionally substituted C₁₋₆alkyl; and
R¹⁶ and R¹⁷ are independently hydrogen or optionally substituted C₁₋₆alkyl, or R¹⁶ and
R¹⁷ combine with the nitrogen to which they are attached to form optionally substituted 5-6 membered heterocycloalkyl.

25. The p53-activating agent for use according to any one of claims 19 to 24, wherein said BRAF-inhibiting agent is a compound selected from the group consisting of N-(3-{[5-(4-chlorophenyl)-1H-pyrrolo[2,3-b]pyridin-3-yl]carbonyl}-2,4-difluorophenyl) propane-1-sulfonamide; N-{3-(5-(2-aminopyrimidin-4-yl)-2-tert-butyl-1,3-thiazol-4-yl]-2-fluorophenyl}-2,6-difluorobenzenesulfonamide; 4-[4-[[4-chloro-3-(trifluoromethyl)phenyl]carbamoylamino]phenoxy]-N-methyl-pyridine-2-carboxamide; N-[3-(5-chloro-1H-pyrrolo[2,3-b]pyridine-3-carbonyl)-2,4-difluorophenyl]propane-1-sulfonamide; (E)-2,3-Dihydro-5-[1-(2-hydroxyethyl)-3-(4-pyridinyl)-1H-pyrazol-4-yl]-1H-inden-1-one oxime; methyl [(2S)-1-{[4-(3-{5-chloro-2-fluoro-3-[(methylsulfonyl)amino]phenyl}-1-isopropyl-1H-pyrazol-4-yl)-2-pyrimidinyl]amino}-2-propanyl]carbamate; and 1-methyl-5-[[2-[5-(trifluoromethyl)-1 H-imidazol-2-yl]-4-pyridyl]oxy]-N-[4-(trifluoromethyl)phenyl]benzimidazol-2-amine.

26. The p53-activating agent for use according to any one of claims 19 to 25, wherein said p53-activating agent is 2-hydroxymethyl-2-methoxymethylazabicyclo[2.2.2]octan-3-one (PRIMA-1^{Met}) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is SCH529074 (Demma M, et al., 2010, J Biol Chem. 285(14):10198-10212) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-inhibiting agent is N-(3-{[5-(4-ch lorophenyl)-1H-pyrrolo[2,3-b]pyridin-3-yl]carbonyl}-2,4-difluorophenyl)-propane-1-sulfonamide (vemurafenib); or
wherein said p53-activating agent is NSC319726 (Yu X. et al., 2012, Cancer Cell. 15;21(5):614-25) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib).

27. The p53-activating agent for use according to any one of claims 19 to 26, wherein said p53-activating agent and said BRAF-inhibiting agent are comprised in a composition or in a kit of parts, preferably in a pharmaceutical composition or in a pharmaceutical kit of parts.

28. The p53-activating agent for use according to any one of claims 19 to 26, wherein said p53-activating agent may be administered simultaneously or sequentially with said BRAF-inhibiting agent and with a MEK-inhibiting agent capable of inhibiting activity of mitogen-activated protein kinase kinase 1 (MEK 1) and/or mitogen-activated protein kinase kinase 2 (MEK2).

29. The p53-activating agent for use according to claim 28, wherein said p53-activating agent said BRAF-inhibiting agent and said MEK-inhibiting agent are comprised in a composition or in a kit of parts, preferably in a pharmaceutical composition or in a pharmaceutical kit of parts.

30. The p53-activating agent for use according to any one of claims 19 to 29, wherein the melanoma comprises expression of BRAF comprising an activating mutation, preferably wherein the melanoma comprises (a) cell(s) comprising expression of ^{V600E/K}BRAF.

31. The p53-activating agent for use according to any one of claims 19 to 30, wherein the melanoma comprises (a) cell(s) with intrinsic or acquired resistance to said BRAF-inhibiting agent.

32. A p53-activating agent capable of transferring wild-type tumor protein p53 (p53) from an inactive conformation into an active conformation capable of inducing apoptosis, for use in the treatment of melanoma resistant to N-(3-{[5-(4-chlorophenyl)-1H-pyrrolo[2,3-b]pyridin-3-yl]carbonyl}-2,4-difluorophenyl)-propane-1-sulfonamide (vemurafenib).

33. The p53-activating agent for use according to claim 32, wherein said resistance is pre-existing, or is acquired due to (chronic) treatment with N-(3-{[5-(4-chlorophenyl)-1H-pyrrolo[2,3-b]pyridin-3-yl]carbonyl}-2,4-difluorophenyl)-propane-1-sulfonamide (vemurafenib).

34. A kit of parts or a composition, preferably a pharmaceutical kit of parts or a pharmaceutical composition, comprising a p53-activating agent capable of transferring wild-type p53 from an inactive conformation thereof into an active conformation capable of inducing apoptosis and a BRAF-inhibiting agent capable of inhibiting activity of BRAF comprising an activating mutation, for use in treating melanoma.

35. The kit of parts or the composition according to claim 33, wherein said p53-activating agent is 2-hydroxymethyl-2-methoxymethylazabicyclo[2.2.2]octan-3-one (PRIMA-1^{Met}) and said BRAF-inhibiting agent is N-(3-{[5-(4-chlorophenyl)-1H-pyrrolo[2,3-b]pyridin-3-yl]carbonyl}-2,4-difluorophenyl) propane-1-sulfonamide (vemurafenib); or wherein said p53-activating agent is CDB3 (Issaeva N et al., 2003, PNAS 100(23):13303-13307) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is SCH529074 (Demma M, et al., 2010, J Biol Chem. 285(14):10198-10212) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is CP-31398 (Luu Y and Li G, 2002, J Invest Dermatol, 119(5):1207-1209; Luu Y et al., 2002 Exp Cell Res, 276(2):214-222.) and said BRAF-in hi biting agent is N-(3-{[5-(4-chlorophenyl)-1H-pyrrolo[2,3-b]pyridin-3-yl]carbonyl}-2,4-difluorophenyl)-propane-1-sulfonamide (vemurafenib); or
wherein said p53-activating agent is NSC319726 (Yu X. et al., 2012, Cancer Cell. 15;21(5):614-25) and said BRAF-inhibiting agent is N-(3-{[5-(4-chlorophenyl)-1H-pyrrolo[2,3-b]pyridin-3-yl]carbonyl}-2,4-difluorophenyl)-propane-1-sulfonamide (vemurafenib).

36. The kit of parts or the composition according to anyone of claim 34 or 35, additionally comprising a MEK-inhibiting agent capable of inhibiting activity of mitogen-activated protein kinase kinase 1 (MEK 1) and/or mitogen-activated protein kinase kinase 2 (MEK2).

37. A method for determining resistance of melanoma to a BRAF-inhibiting agent as defined according to anyone of claims 19 to 33, in a subject, wherein the method comprises the steps of:
(i) preparing a cell culture from a sample of the melanoma obtained from the subject, and
(ii) determining the cytotoxicity of cells of the cell culture to said BRAF-inhibiting agent, wherein the melanoma is intrinsically resistant to said BRAF-inhibiting agent when the IC50 of the cells is at least 10 μM, and wherein the melanoma is sensitive to said BRAF-inhibiting agent when the IC50 of the cells is less than 10 μM.

38. The method according to claim 47, for predicting the development of resistance to said BRAF-inhibiting agent in a melanoma initially sensitive to said BRAF-inhibiting agent, wherein the method further comprises the steps of:
(iii) treating the melanoma cell culture by chronic exposure with increasing concentrations of said BRAF-inhibiting agent during at least about 4 weeks, and
(iv) determining the cytotoxicity of cells of the cell culture to said BRAF-inhibiting agent after said treatment, wherein the melanoma has acquired resistance to the BRAF-inhibiting agent when the IC50 of the cells is at least 10 μM.

39. The method according to claim 47 or 48, wherein the melanoma sample obtained from the subject originates from a metastasis of the subject, for example from skin, lymph node, mucosa, liver, or gastrointestinal tract.

40. A method for predicting responsiveness of melanoma resistant to a BRAF-inhibiting agent as defined according to anyone of claims 19 to 33, to treatment with a p53-activating agent as defined according to anyone of claims 19 to 33 in combination said BRAF-inhibiting agent in a subject, comprising the steps of:
(i) preparing a cell culture from a sample of the melanoma obtained from the subject,
(ii) determining the expression of one or more of p53, Phosphatase and tensin homolog (PTEN), and phospho-Protein kinase B (pAKT) in cells of the cell culture, and
(iii) predicting that the melanoma is responsive to treatment with said p53-activating agent in combination with said BRAF-inhibiting agent, if the cells express low p53, low PTEN, and/or high pAkt compared with expression of the respective proteins in cells of a cell culture prepared from a melanoma sensitive to said BRAF-inhibiting agent.

41. A method for predicting responsiveness of melanoma to treatment with a p53-activating agent as defined according to anyone of claims 19 to 33 in combination with a BRAF-inhibiting agent defined according to anyone of claims 19 to 33 in a subject, comprising the steps of:
(i) preparing a cell culture from a sample of the melanoma obtained from the subject,
(ii) administering said p53-activating agent in combination with said BRAF-inhibiting agent,
(iii) administering said BRAF-inhibiting agent alone as a control treatment,
(iv) determining the cytotoxicity of cells of the cell culture to said p53 activating agent and said BRAF-inhibiting agent and to said BRAF-inhibiting agent alone, and
(v) predicting that the melanoma is responsive to treatment with said p53-activating agent in combination with said BRAF-inhibiting agent, if the cytotoxicity of said p53-activating agent in combination with said BRAF-inhibiting agent to the cells is higher compared to the cytotoxicity of cells treated with said BRAF-inhibiting agent alone.

42. The method of treatment according to anyone of claims 1 to 18, wherein the subject is a subject with a melanoma responsive to treatment with the p53-activating agent in combination with the BRAF-inhibiting agent, as determined by the method according to claim 40 or 41

43. The p53-activating agent for use according to any one of claims 19 to 33, wherein the subject is a subject with a melanoma responsive to treatment with the p53-activating agent in combination with the BRAF-inhibiting agent, as determined by the method according to claim 40 or 41.
